(19) United States
(12) Patent Application Publication
(10) Pub. No.: US 2026/0207790 A1
(43) Pub. Date: Jul. 23, 2026

(54) BIOINKS AND METHODS OF SYNTHESIZING, CHARACTERIZING, AND DEPLOYING BIOINKS

(71) Applicant: VOXCELL BIOINNOVATION INC., VICTORIA (CA)

(72) Inventors: Karolina Valente, Victoria (CA); Charlotte Ellis, Victoria (CA); Benjamin Rampado, Lake Country (CA); Kevin Vos, Victoria (CA); Geneviève Boice, Victoria (CA)

(73) Assignee: VOXCELL BIOINNOVATION INC., VICTORIA, BC (CA)

(21) Appl. No.: 19/139,339

(22) PCT Filed: Dec. 15, 2023
(86) PCT No.: PCT/CA2023/051679

§ 371 Date: Jun. 14, 2025

Related U.S. Application Data
(60) Provisional application No. 63/432,988, filed on Dec. 15, 2022.

(51) Int. Cl. A61K 49/00; C07K 1/107; C07K 1/12; C07K 14/78; C08B 37/08; G01N 3/42

(52) U.S. Cl. CPC A61K 49/0054; C07K 1/1077; C07K 1/122; C07K 14/78; C08B 37/0072; G01N 3/42

(57) ABSTRACT

Bioinks and methods of synthesizing, characterizing, and deploying bioinks are described. Compounds and methods of making the same are described. Method of synthesizing pH-responsive polymers are described. Different types of bioinks are described. For example, the described bioinks may comprise: one or more of the pH-responsive polymers; a photoinitiator; and a solvent. Methods preparing bioinks are described. Methods of determining characteristics of bioinks are described. Related apparatus, kits, methods, and systems also are described.

### BACKGROUND

### Field of Invention

Aspects of this disclosure relate generally to bioinks and methods of synthesizing, characterizing, and deploying bioinks. Particular aspects relate to pH-responsive polymers and bioinks comprising one or more pH-responsive polymers, such as tissue-specific and/or universal bioinks.

### Description of Related Art

Hydrogels are three-dimensional (3D) networks of polymeric materials (natural or synthetic) capable of absorbing a considerable amount of water and swelling in an aqueous medium, while maintaining physical integrity due to the presence of hydrophilic functional groups (—OH, —COOH, —SO₃H, and —NH₂). Hydrogels have been commonly applied in tissue engineering and as drug delivery systems due to their biocompatibility and resemblance to a natural tissue.

Gelatin is a natural polymer originated from collagen, derived through a denaturation process. It is an important protein that contributes to the stiffness of soft tissues. Depending on the denaturation process (acidic or basic treatment), a Gelatin Type A or a Gelatin Type B may be obtained, respectively. Gelatin methacryloyl (commonly known as “GelMA”) is a semi-synthetic biomaterial prepared by adding methacryloyl, methacrylate, and/or methacrylamide groups to a gelatin backbone after reaction with methacrylic anhydride or glycidyl methacrylate. The presence of arginine-glycine-aspartic acid (“RGD”) sequences on GelMA due to its gelatin backbone promotes cell attachments useful for biomedical materials.

Other naturally occurring polymers commonly used for biomedical applications may include collagen, elastin, and hyaluronic acid, each of which is a component of extracellular matrices of soft tissues providing key structural and biochemical characteristics. Collagen is the most abundant structural protein in the human body and provides high tensile strength to tissues. Collagen also plays a crucial role in the regeneration of soft tissues. Elastin is a major component of human vasculature and contributes to the regulation of diffusion through capillary walls. It also exhibits a low elastic modulus, therefore contributing to stretch and recoil of tissues. Hyaluronic acid is a highly hydrophilic polysaccharide which plays a key structural role in the extracellular matrices of soft tissues. Hyaluronic acid is responsible for a vast range of functions within tissues, including regulation of cell proliferation and differentiation.

### SUMMARY

Aspects of bioinks and methods of synthesizing, characterizing, and deploying bioinks are described in this disclosure. One aspect of this disclosure is a compound of formula (I). According to this aspect, for example, the compound of formula (I) may comprise:

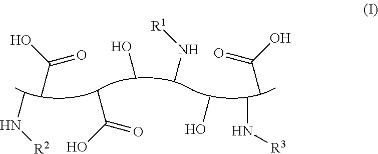

wherein:
the compound comprises a collagen backbone;
R¹, R², and R³ are selected from a group consisting of:

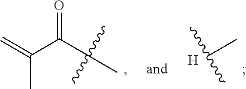

at least one of R¹, R², and R³ is not

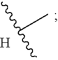

and
“ [symbol] ” indicates the point of attachment.

According to this aspect, the compound of formula (I) may comprise

| where: | |
|---|---|
| R¹ is | 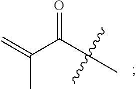 [SMILES: *C(=O)C(=C)C] |
| R² is | 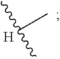 |
| and | |
| R³ is | 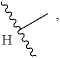 |
| or where: | |
| R¹ is | 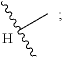 |
| R² is | 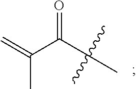 [SMILES: *C(=O)C(=C)C] |
| and | |
| R³ is | 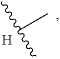 |
| or where: | |
| R¹ is | 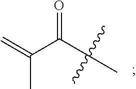 [SMILES: *C(=O)C(=C)C] |
| R² is | 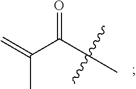 [SMILES: *C(=O)C(=C)C] |
| and | |
| R³ is | 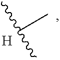 |
| or where: | |
| R¹ is | 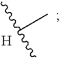 |
| R² is | 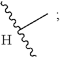 |
| and | |
| R³ is | 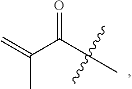 [SMILES: *C(=O)C(=C)C] |
| or where: | |
| R¹ is | 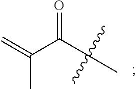 [SMILES: *C(=O)C(=C)C] |
| R² is | 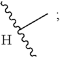 |
| and | |
| R³ is | 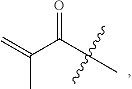 [SMILES: *C(=O)C(=C)C] |
| or where: | |
| R¹ is | 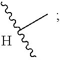 |
| R² is | 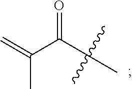 [SMILES: *C(=O)C(=C)C] |
| and | |
| R³ is | 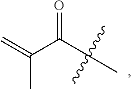 [SMILES: *C(=O)C(=C)C] |
| or where: | |
| R¹ is | 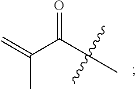 [SMILES: *C(=O)C(=C)C] |
| R² is | 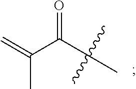 [SMILES: *C(=O)C(=C)C] |
| and | |
| R³ is | 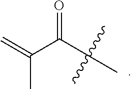 [SMILES: *C(=O)C(=C)C] |

and

Another aspect of this disclosure is a method of synthesizing a pH-responsive polymer. According to this aspect, the method may comprise: maintaining a vessel at a reaction temperature; mixing a collagen with a basic water in the vessel to form a first solution having a target pH; stirring the first solution at the reaction temperature while: beginning a reaction by adding methacrylic anhydride to the vessel to form a second solution; conducting the reaction for a first time period in the absence of light while maintaining the second solution at the target pH by obtaining a measured pH of the second solution at intervals during the first time period, determining a difference between the measured pH and the target pH at each one of the intervals, and adding a base to the vessel at each one of the intervals so that the measured pH equals the target pH; and removing unreacted methacrylic anhydride from the second solution.

Maintaining the vessel at the reaction temperature may comprise placing the vessel in a temperature bath. The reaction temperature may be between approximately 18° C. and approximately 25° C. The target pH may be 10. Mixing the collagen with the basic water may comprise: dissolving the collagen in an acetic acid in the vessel; adding water and sodium hydroxide to the vessel; and stirring contents of the vessel to form the first solution. The collagen may comprise a Type 1 rat tail collagen as a 5 mg/mL solution. The acetic acid may comprise a 20 mM aqueous acetic acid. The sodium hydroxide may comprise a 2 M sodium hydroxide. Mixing the collagen with the basic water may comprise adding the basic water to the first solution dropwise at a flowrate of approximately 1 mL/min. The method may comprise stirring the first solution at approximately 600 rpm. The method may comprise adding the methacrylic anhydride dropwise at a flowrate of approximately 100 μL/min. The method may comprise stirring the second solution at approximately 600 rpm.

The basic water may have a pH of approximately 10. The base may have a pH of approximately 14. The first time period may be approximately 2 hours. Each one of the intervals may be approximately 10 minutes. The intervals may occur multiple times during each hour of the first time period. Removing the unreacted methacrylic anhydride may comprise: dialyzing the second solution; freezing the second solution; and lyophilizing the second solution. Dialyzing the second solution may comprise: transferring the second solution to a dialysis membrane; and removing the unreacted methacrylic anhydride by dialysis with the dialysis membrane. Removing the unreacted methacrylic anhydride with the dialysis membrane may comprise dialyzing the second solution against water, with the dialysis membrane, for a dialysis time period at a dialysis temperature. The dialysis temperature may be 30° C. The dialysis temperature may be approximately 30° C. The dialysis time period may be less than or equal to approximately 7 days. The dialysis time period may be at least approximately 5 days. The method may comprise adjusting the dialysis time period relative to a volume of the second solution. Freezing the second solution may comprise exposing the second solution to a freezing temperature equal to approximately −196° C. to create a frozen second solution. Lyophilizing the second solution may comprise placing the frozen second solution in a lyophilizer for a lyophilization time period. The method may comprise outputting a dry product of methacrylated collagen polymer from the lyophilizer. The lyophilization time period may be less than or equal to approximately 7 days. The lyophilization time period may be at least approximately 3 days. The method may comprise adjusting the lyophilization time period relative to a volume of the frozen second solution.

Another aspect of this disclosure is a method of synthesizing a pH-responsive polymer. According to this aspect, the method may comprise: maintaining a vessel at a reaction temperature; mixing a collagen with an acidic water in the vessel to form a first solution; adding a neutral water to the vessel to form a second solution having a target pH; stirring the second solution at the reaction temperature while beginning a reaction by adding methacrylic anhydride to the vessel to form a third solution, and conducting the reaction in the absence of light; and removing unreacted methacrylic anhydride from the third solution.

Maintaining the vessel at the reaction temperature may comprise placing the vessel in a temperature bath. Maintaining the vessel at the reaction temperature may comprise placing the vessel in a temperature-controlled environment. The reaction temperature may be between approximately 3° C. and approximately 6° C. The target pH may be 7.5. The acidic water may comprise a hydrochloric acid and mixing the mixing the collagen with the acidic water may comprise: dissolving the collagen in the hydrochloric acid; and stirring the collagen and the acidic water in the vessel for a first time period to form the first solution. The collagen may comprise a type 1 rat tail collagen. The hydrochloric acid may comprise a 10 mM aqueous hydrochloric acid. The first time period may be approximately 24 hours. Adding the neutral water may comprise: adding a sodium hydrogen phosphate to the first solution; and stirring the sodium hydrogen phosphate and the first solution in the vessel to form the second solution. The sodium hydrogen phosphate may comprise a 0.2 M aqueous sodium hydrogen phosphate dibasic. The method may comprise stirring the second solution in the vessel at a stir rate of approximately 600 rpm.

The method may comprise adding the methacrylic anhydride at a rate of approximately 100 μL/min. The method may comprise conducting the reaction for a time period of approximately 8 hours. Removing the unreacted methacrylic anhydride may comprise: dialyzing the third solution; freezing the third solution; and lyophilizing the third solution. Dialyzing the third solution may comprise: transferring the third solution to a dialysis membrane; and removing the unreacted methacrylic anhydride by dialysis with the dialysis membrane. Removing the unreacted methacrylic anhydride with the dialysis membrane may comprise dialyzing the third solution against 10 mM HCl, with the dialysis membrane, for a dialysis time period at a dialysis temperature. The dialysis temperature may be 4 C. The dialysis temperature may be between 2° C. and 8° C. The dialysis time period may equal to or less than approximately 3 days. The dialysis time period may be at least approximately 2 days. Freezing the third solution may comprise exposing the third solution to a freezing temperature equal to approximately −196° C. to create a frozen third solution. Lyophilizing the third solution may comprise placing the frozen third solution in a lyophilizer for a lyophilization time period. The method may comprise outputting a dry product of methacrylated collagen polymer from the lyophilizer. The lyophilization time period may be less than or equal to approximately 7 days. The lyophilization time period may be at least approximately 3 days. The method may comprise adjusting the lyophilization time period relative to a volume of the frozen third solution.

Another aspect of this disclosure is a compound of formula (II). According to this aspect, for example, the compound of formula (II) may comprise:

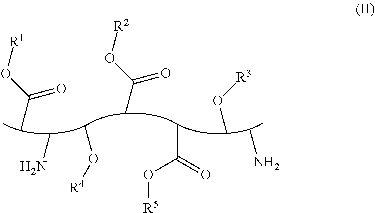

wherein:
the compound comprises an elastin backbone;
R¹, R², R³, R⁴, and R⁵ are selected from a group consisting of:

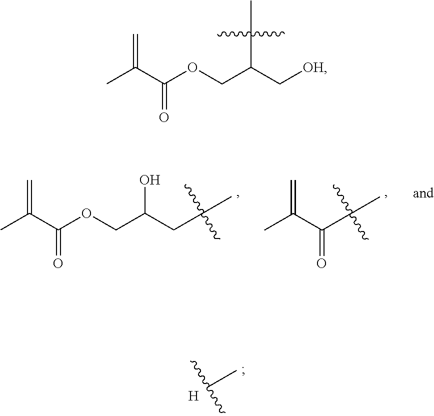

at least one of R¹, R², R³, R⁴, and R⁵ is not

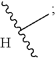

and
“ [symbol] ” indicates the point of attachment.

According to this aspect, the compound of formula (II) may comprise

| where: | |
|---|---|
| R¹ is | 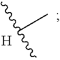 |
| R² is | 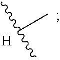 |
| R³ is | 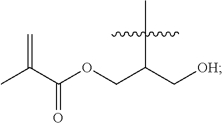 [SMILES: *C(CO)COC(=O)C(=C)C] |
| R⁴ is | 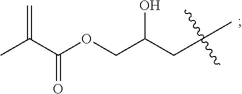 [SMILES: *CC(O)COC(=O)C(=C)C] |
| and | |
| R⁵ is | 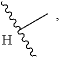 |
| or where: | |
| R¹ is | 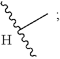 |
| R² is | 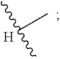 |
| R³ is | 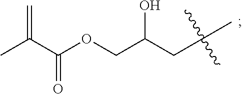 [SMILES: *CC(O)COC(=O)C(=C)C] |
| R⁴ is | 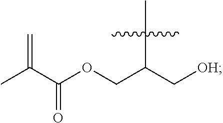 [SMILES: *C(CO)COC(=O)C(=C)C] |
| and | |
| R⁵ is | 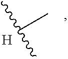 |
| or where: | |
| R¹ is | 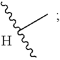 |
| R² is | 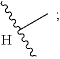 |
| R³ is | 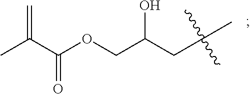 [SMILES: *CC(O)COC(=O)C(=C)C] |
| R⁴ is | 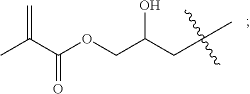 [SMILES: *CC(O)COC(=O)C(=C)C] |
| and | |
| R⁵ is | 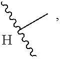 |
| or where: | |
| R¹ is | 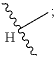 |
| R² is | 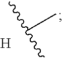 |
| R³ is | 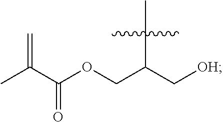 [SMILES: *C(CO)COC(=O)C(=C)C] |
| R⁴ is | 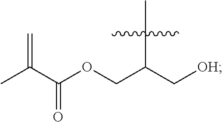 [SMILES: *C(CO)COC(=O)C(=C)C] |
| and | |
| R⁵ is | 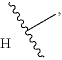 |
| or where: | |
| R¹ is | 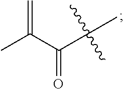 [SMILES: *C(=O)C(=C)C] |
| R² is | 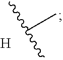 |
| R³ is | 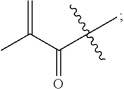 [SMILES: *C(=O)C(=C)C] |
| R⁴ is | 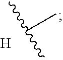 |
| and | |
| R⁵ is | 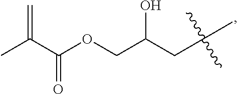 [SMILES: *CC(O)COC(=O)C(=C)C] |
| or where: | |
| R¹ is | 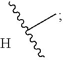 |
| R² is | 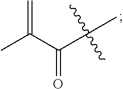 [SMILES: *C(=O)C(=C)C] |
| R³ is | 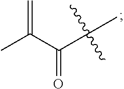 [SMILES: *C(=O)C(=C)C] |
| R⁴ is | 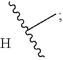 |
| and | |
| R⁵ is | 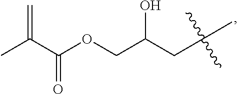 [SMILES: *CC(O)COC(=O)C(=C)C] |
| or where: | |
| R¹ is | 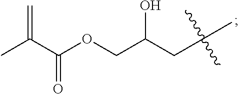 [SMILES: *CC(O)COC(=O)C(=C)C] |
| R² is | 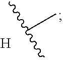 |
| R³ is | 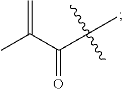 [SMILES: *C(=O)C(=C)C] |
| R⁴ is | 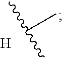 |
| and | |
| R⁵ is | 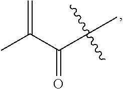 [SMILES: *C(=O)C(=C)C] |
| or where: | |
| R¹ is | 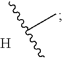 |
| R² is | 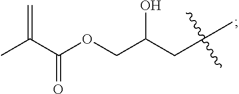 [SMILES: *CC(O)COC(=O)C(=C)C] |
| R³ is | 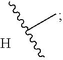 |
| R⁴ is | 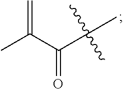 [SMILES: *C(=O)C(=C)C] |
| and | |
| R⁵ is | 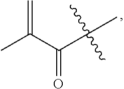 [SMILES: *C(=O)C(=C)C] |
| or where: | |
| R¹ is | 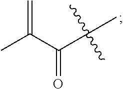 [SMILES: *C(=O)C(=C)C] |
| R² is | 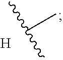 |
| R³ is | 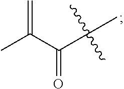 [SMILES: *C(=O)C(=C)C] |
| R⁴ is | 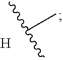 |
| and | |
| R⁵ is | 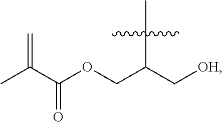 [SMILES: *C(CO)COC(=O)C(=C)C] |
| or where: | |
| R¹ is | 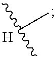 |
| R² is | 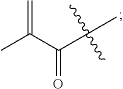 [SMILES: *C(=O)C(=C)C] |
| R³ is | 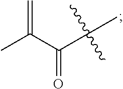 [SMILES: *C(=O)C(=C)C] |
| R⁴ is | 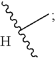 |
| and | |
| R⁵ is | 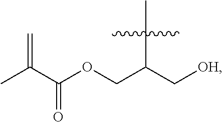 [SMILES: *C(CO)COC(=O)C(=C)C] |
| or where: | |
| R¹ is | 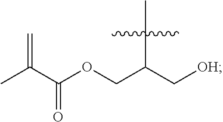 [SMILES: *C(CO)COC(=O)C(=C)C] |
| R² is | 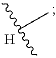 |
| R³ is | 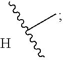 |
| R⁴ is | 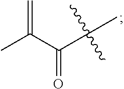 [SMILES: *C(=O)C(=C)C] |
| and | |
| R⁵ is | 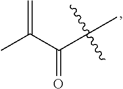 [SMILES: *C(=O)C(=C)C] |
| or where: | |
| R¹ is | 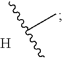 |
| R² is | 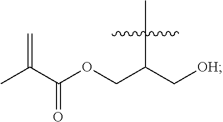 [SMILES: *C(CO)COC(=O)C(=C)C] |
| R³ is | 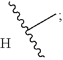 |
| R⁴ is | 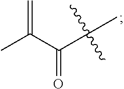 [SMILES: *C(=O)C(=C)C] |
| and | |
| R⁵ is | 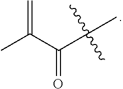 [SMILES: *C(=O)C(=C)C] |

Another aspect of this disclosure is a method of synthesizing a pH-responsive polymer. According to this aspect, the method may comprise: maintaining a vessel at a reaction temperature; mixing a hydrolyzed elastin with a basic water in the vessel to form a first solution having a first pH; stirring the first solution at the reaction temperature while: beginning a reaction by adding methacrylic anhydride to the vessel to form a second solution; conducting the reaction for a first time period in the absence of light while maintaining the second solution at the target pH by obtaining a measured pH of the second solution at intervals during the first time period, determining a difference between the measured pH and the target pH at each one of the intervals, and adding a base to the vessel at each one of the intervals so that the measured pH equals the target pH; and removing unreacted methacrylic anhydride from the second solution.

Maintaining the vessel at the reaction temperature may comprise placing the vessel in a temperature bath. The reaction temperature may be between approximately 18° C. and approximately 25° C. The target pH may be 10. Mixing the hydrolyzed elastin with the basic water may comprise: dissolving the hydrolyzed elastin in the basic water in the vessel; adding water and sodium hydroxide to the vessel; and stirring the hydrolyzed elastin, the basic water, the water, and the sodium hydroxide in the vessel to form the first solution. Mixing the hydrolyzed elastin with the basic water may comprise adding the basic water to the first solution dropwise at a flowrate of approximately 1 mL/min. The method may comprise one or both of: stirring the first solution at approximately 600 rpm; and stirring the second solution at approximately 600 rpm. The method may comprise adding the methacrylic anhydride dropwise at a flowrate of approximately 100 μL/min. The basic water may have a pH of approximately 10. The base may have a pH of approximately 14. The base may comprise 2 M sodium hydroxide. The first time period may be approximately 2 hours. Each one of the intervals may be approximately 10 minutes. The intervals may occur multiple times during each hour of the first time period.

Removing the unreacted methacrylic anhydride may comprise: dialyzing the second solution; freezing the second solution; and lyophilizing the second solution. Dialyzing the second solution may comprise: transferring the second solution to a dialysis membrane; and removing the unreacted methacrylic anhydride by dialysis with the dialysis membrane. Removing the unreacted methacrylic anhydride with the dialysis membrane may comprise dialyzing the second solution against water, with the dialysis membrane, for a dialysis time period at a dialysis temperature. The dialysis temperature may be 30° C. The dialysis temperature may be approximately 30° C. The dialysis time period may be less than or equal to approximately 7 days. The dialysis time period may be at least approximately 5 days. The method may comprise adjusting the dialysis time period relative to a volume of the second solution. Freezing the second solution may comprise exposing the second solution to a freezing temperature equal to approximately −196° C. to create a frozen second solution. Lyophilizing the second solution may comprise placing the frozen second solution in a lyophilizer for a lyophilization time period. The method may comprise outputting a dry product of methacrylated hydrolyzed elastin from the lyophilizer. The lyophilization time period may be less than or equal to approximately 7 days. The lyophilization time period may be at least approximately 3 days. The method may comprise adjusting the lyophilization time period relative to a volume of the frozen second solution.

The method may comprise synthesizing the hydrolyzed elastin. Synthesizing the hydrolyzed elastin may comprise: suspending elastin in an aqueous oxalic acid solution to form a suspension; refluxing the suspension for an initial time period at an initial temperature; cooling the suspension to a cooled temperature after the initial time period; centrifuging the suspension for a subsequent time period at the cooled temperature; collecting a supernatant comprising the hydrolyzed elastin from the suspension; collecting a precipitate comprising elastin from the suspension; repeating the preceding steps with the precipitate to collect additional amounts of the hydrolyzed elastin; and purifying the hydrolyzed elastin collected during the preceding steps. Suspending the elastin may comprise suspending 5 g of the elastin in 40 mL of a 0.25 M aqueous oxalic acid solution. The initial time period may be approximately one hour. The initial temperature may be approximately 100° C. The cooled temperature may be approximately 4° C. The method may comprise centrifuging the suspension at 3750 rpm. Purifying the hydrolyzed elastin may comprise: dialyzing the hydrolyzed elastin collected in the method; freezing the hydrolyzed elastin collected in the method; and lyophilizing the hydrolyzed elastin collected in the method.

Another aspect of this disclosure is a compound of formula (III). According to this aspect, for example, the compound of formula (III) may comprise:

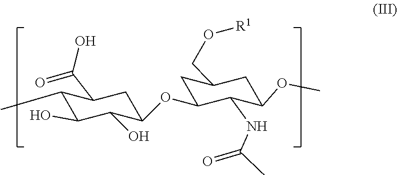

wherein:
the compound comprises a hyaluronic acid backbone;
R¹ is selected from a group consisting of:

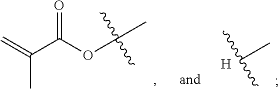

and
“ [symbol] ” indicates the point of attachment.

According to this aspect, the compound of formula (III) may comprise

| where: | |
|---|---|
| R¹ is | 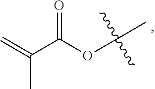 [SMILES: *OC(=O)C(=C)C] |
| or where: | |
| R¹ is | 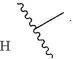 |

Another aspect of this disclosure is a method of synthesizing a pH-responsive polymer. According to this aspect, the method may comprise: maintaining a vessel at a reaction temperature; mixing a hyaluronic acid with a basic water in the vessel for a first time period to form a first solution having a target pH; stirring the first solution at the reaction temperature while beginning a reaction by adding methacrylic anhydride to the vessel to form a second solution, adding a base to the vessel to maintain the second solution at the target pH, and conducting the reaction in the absence of light for a second time period while maintaining the second solution at the reaction temperature; and removing unreacted methacrylic anhydride from the second solution.

Maintaining the vessel at the reaction temperature may comprise placing the vessel in a temperature bath. The reaction temperature may be approximately 4° C. The reaction temperature may be between approximately 0° C. and approximately 10° C. The hyaluronic acid may comprise a hyaluronic acid sodium salt. Mixing the hyaluronic acid with the basic water comprises adding the basic water to the first solution dropwise at a flowrate of approximately 1 mL/min. The first time period may be approximately 24 hours. The method may comprise stirring the first solution at approximately 600 rpm. The method may comprise adding the methacrylic anhydride dropwise at a flowrate of approximately 100 μL/min. The method may comprise stirring the second solution at approximately 600 rpm. The target pH may be 10. The basic water may have a pH of approximately 10. The base may have a pH of approximately 14. The base may comprise 5 M sodium hydroxide. The second time period may be approximately 24 hours.

Removing the unreacted methacrylic anhydride may comprise: dialyzing the second solution; freezing the second solution; and lyophilizing the second solution. Dialyzing the second solution may comprise: transferring the second solution to a dialysis membrane; and removing the unreacted methacrylic anhydride by dialysis with the dialysis membrane. Removing the unreacted methacrylic anhydride with the dialysis membrane may comprise dialyzing the second solution against water, with the dialysis membrane, for a dialysis time period at a dialysis temperature. The dialysis temperature may be 40° C. The dialysis temperature may be approximately 40° C. The dialysis time period may be less than or equal to approximately 7 days. The dialysis time period may be at least approximately 5 days. The method may comprise adjusting the dialysis time period relative to a volume of the second solution. Freezing the second solution may comprise exposing the second solution to a freezing temperature equal to approximately −196° C. to create a frozen second solution. Lyophilizing the second solution may comprise placing the frozen second solution in a lyophilizer for a lyophilization time period. The method may comprise outputting a dry product of methacrylated hyaluronic acid polymer from the lyophilizer. The lyophilization time period may be less than or equal to approximately 7 days. The lyophilization time period may be at least approximately 3 days. The method may comprise adjusting the lyophilization time period relative to a volume of the frozen second solution.

Another aspect of this disclosure is a method of synthesizing a pH-responsive polymer. According to this aspect, the method may comprise: maintaining a vessel at a reaction temperature; mixing a hyaluronic acid with a water in the vessel for a first time period to form a first solution; adding a basic water to the vessel to form a second solution having a target pH; stirring the second solution in the vessel at the reaction temperature while beginning a reaction by adding methacrylic anhydride to the vessel to form a third solution, adding a base to the vessel to maintain the third solution at a target pH, and conducting the reaction in the absence of light for a second time period; and removing unreacted methacrylic anhydride from the third solution.

Maintaining the vessel at the reaction temperature may comprise placing the vessel in a temperature bath. Maintaining the vessel at the reaction temperature may comprise placing the vessel in a temperature-controlled environment. The reaction temperature may be approximately 4° C. The reaction temperature may be between approximately 2° C. and approximately 10° C. Mixing the hyaluronic acid with the water may comprise: dissolving the hyaluronic acid in the water in the vessel; and stirring the hyaluronic acid and the water in the vessel for a first time period to form the first solution. The hyaluronic acid may comprise a hyaluronic acid sodium salt. The first time period may be approximately 24 hours. Adding the basic water may comprise: adding sodium hydroxide to the first solution; and stirring the sodium hydroxide and the first solution in the vessel to form the second solution. The sodium hydroxide may comprise 5M aqueous sodium hydroxide. The target pH may be 8.5. The method may comprise stirring the second solution at approximately 600 rpm. The method may comprise adding the methacrylic anhydride at a rate of approximately 100 μL/min. The base may have a pH of approximately 14. The base may comprise 5M sodium hydroxide. The method may comprise conducting the reaction for a time period of approximately 24 hours.

Removing the unreacted methacrylic anhydride may comprise: dialyzing the third solution; freezing the third solution; and lyophilizing the third solution. Dialyzing the third solution may comprise: transferring the third solution to a dialysis membrane; and removing the unreacted methacrylic anhydride by dialysis with the dialysis membrane. Removing the unreacted methacrylic anhydride with the dialysis membrane may comprise dialyzing the third solution against water, with the dialysis membrane, for a dialysis time period at a dialysis temperature. The dialysis temperature may be 40° C. The dialysis temperature may be approximately 40° C. The dialysis time period may be equal to or less than approximately 3 days. The dialysis time period may be at least approximately 2 days. Freezing the third solution may comprise exposing the third solution to a freezing temperature equal to approximately −196° C. to create a frozen third solution. Lyophilizing the third solution may comprise placing the frozen third solution in a lyophilizer for a lyophilization time period. The method may comprise outputting a dry product of methacrylated hyaluronic acid polymer from the lyophilizer. The lyophilization time period may be less than or equal to approximately 7 days. The lyophilization time period may be at least approximately 3 days. The method may comprise adjusting the lyophilization time period relative to a volume of the frozen third solution.

Another aspect of this disclosure is a method of synthesizing a pH-responsive polymer. According to this aspect, the method may comprise: maintaining a vessel at a reaction temperature; mixing a hyaluronic acid with a buffer in the vessel to form a first solution having a target pH; stirring the first solution at the reaction temperature while beginning a reaction by adding methacrylic anhydride to the vessel to form a second solution, and conducting the reaction in the absence of light; and removing unreacted methacrylic anhydride from the second solution.

Maintaining the vessel at the reaction temperature may comprise placing the vessel in a temperature bath. Maintaining the vessel at the reaction temperature may comprise placing the vessel in a temperature-controlled environment. The reaction temperature may be approximately 4° C. The reaction temperature may be between approximately 2° C. and approximately 10° C. The target pH may be 8.5. The buffer may comprise a sodium carbonate buffer and mixing the hyaluronic acid with the buffer may comprise: dissolving the hyaluronic acid in the sodium carbonate buffer in the vessel; and stirring the hyaluronic acid and the sodium carbonate buffer in the vessel for a first time period to form the first solution.

The hyaluronic acid may comprise a hyaluronic acid sodium salt. The sodium carbonate buffer may comprise a bicarbonate aqueous solution. The first time period may be approximately 24 hours. The method may comprise stirring the first solution at approximately 600 rpm. The method may comprise adding the methacrylic anhydride at a rate of approximately 100 μL/min. The method may comprise conducting the reaction for a time period of approximately 24 hours. Removing the unreacted methacrylic anhydride may comprise: dialyzing the second solution; freezing the second solution; and lyophilizing the second solution. Dialyzing the second solution may comprise: transferring the second solution to a dialysis membrane; and removing the unreacted methacrylic anhydride by dialysis with the dialysis membrane.

Removing the unreacted methacrylic anhydride with the dialysis membrane may comprise dialyzing the second solution against water, with the dialysis membrane, for a dialysis time period at a dialysis temperature. The dialysis temperature may be 40° C. The dialysis temperature may be approximately 40° C. The dialysis time period may be equal to or less than approximately three days. The dialysis time period may be at least approximately 2 days. Freezing the second solution may comprise exposing the second solution to a freezing temperature equal to approximately −196° C. to create a frozen second solution. Lyophilizing the second solution may comprise placing the frozen second solution in a lyophilizer for a lyophilization time period. The method may comprise outputting a dry product of methacrylated hyaluronic acid polymer from the lyophilizer. The lyophilization time period may be less than or equal to approximately 7 days. The lyophilization time period may be at least approximately 3 days. The method may comprise adjusting the lyophilization time period relative to a volume of the frozen second solution.

Another aspect of this disclosure is a compound of formula (IV). According to this aspect, for example, the compound of formula (IV) may comprise:

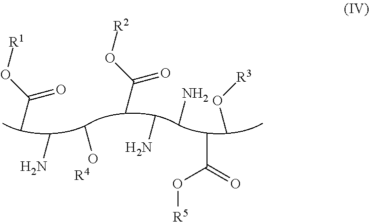

wherein:
the compound comprises a gelatin backbone;
R¹, R², R³, R⁴, and R⁵ are selected from a group consisting of:

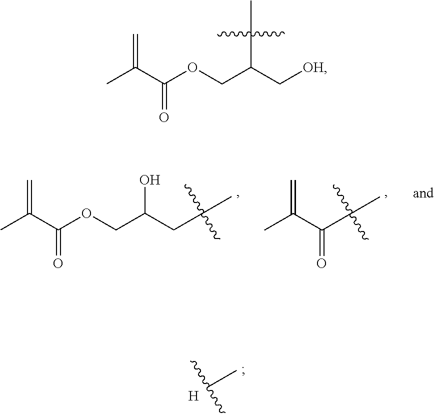

at least one of R¹, R², R³, R⁴, and R⁵ is not

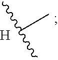

and
“ [symbol] ” indicates the point of attachment.

According to this aspect, the compound of formula (III) may comprise

| where: | |
|---|---|
| R¹ is | 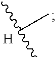 |
| R² is | 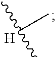 |
| R³ is | 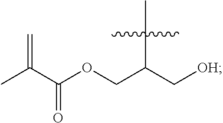 [SMILES: *C(CO)COC(=O)C(=C)C] |
| R⁴ is | 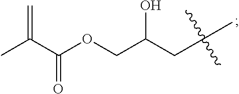 [SMILES: *CC(O)COC(=O)C(=C)C] |
| and | |
| R⁵ is | 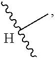 |
| or where: | |
| R¹ is | 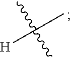 [SMILES: *[H]] |
| R² is | 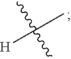 [SMILES: *[H]] |
| R³ is | 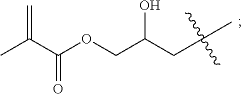 [SMILES: *CC(O)COC(=O)C(=C)C] |
| R⁴ is | 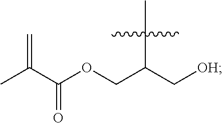 [SMILES: *C(CO)COC(=O)C(=C)C] |
| and | |
| R⁵ is | 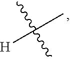 [SMILES: *[H]] |
| or where: | |
| R¹ is | 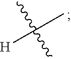 [SMILES: *[H]] |
| R² is | 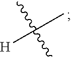 [SMILES: *[H]] |
| R³ is | 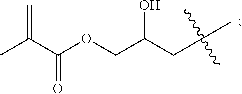 [SMILES: *CC(O)COC(=O)C(=C)C] |
| R⁴ is | 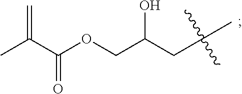 [SMILES: *CC(O)COC(=O)C(=C)C] |
| and | |
| R⁵ is | 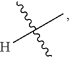 [SMILES: *[H]] |
| or where: | |
| R¹ is | 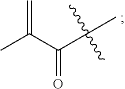 [SMILES: *C(=O)C(=C)C] |
| R² is | 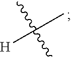 [SMILES: *[H]] |
| R³ is | 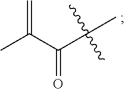 [SMILES: *C(=O)C(=C)C] |
| R⁴ is | 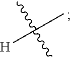 [SMILES: *[H]] |
| and | |
| R⁵ is | 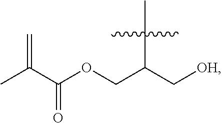 [SMILES: *C(CO)COC(=O)C(=C)C] |
| or where: | |
| R¹ is | 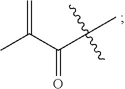 [SMILES: *C(=O)C(=C)C] |
| R² is | 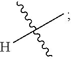 [SMILES: *[H]] |
| R³ is | 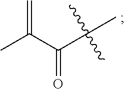 [SMILES: *C(=O)C(=C)C] |
| R⁴ is | 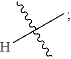 [SMILES: *[H]] |
| and | |
| R⁵ is | 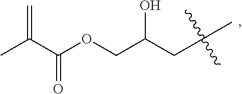 [SMILES: *CC(O)COC(=O)C(=C)C] |
| or where: | |
| R¹ is | 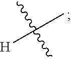 [SMILES: *[H]] |
| R² is | 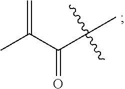 [SMILES: *C(=O)C(=C)C] |
| R³ is | 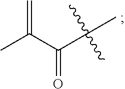 [SMILES: *C(=O)C(=C)C] |
| R⁴ is | 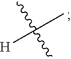 [SMILES: *[H]] |
| and | |
| R⁵ is | 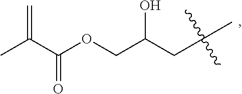 [SMILES: *CC(O)COC(=O)C(=C)C] |
| or where: | |
| R¹ is | 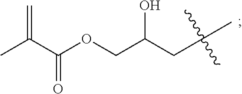 [SMILES: *CC(O)COC(=O)C(=C)C] |
| R² is | 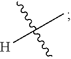 [SMILES: *[H]] |
| R³ is | 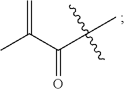 [SMILES: *C(=O)C(=C)C] |
| R⁴ is | 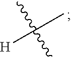 [SMILES: *[H]] |
| and | |
| R⁵ is | 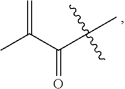 [SMILES: *C(=O)C(=C)C] |
| or where: | |
| R¹ is | 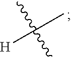 [SMILES: *[H]] |
| R² is | 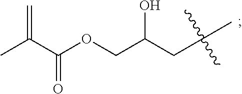 [SMILES: *CC(O)COC(=O)C(=C)C] |
| R³ is | 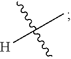 [SMILES: *[H]] |
| R⁴ is | 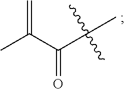 [SMILES: *C(=O)C(=C)C] |
| and | |
| R⁵ is | 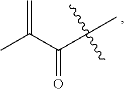 [SMILES: *C(=O)C(=C)C] |
| or where: | |
| R¹ is | 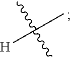 [SMILES: *[H]] |
| R² is | 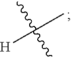 [SMILES: *[H]] |
| R³ is | 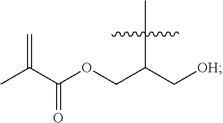 [SMILES: *C(CO)COC(=O)C(=C)C] |
| R⁴ is | 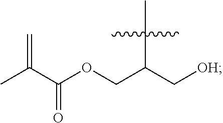 [SMILES: *C(CO)COC(=O)C(=C)C] |
| and | |
| R⁵ is | 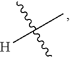 [SMILES: *[H]] |
| or where: | |
| R¹ is | 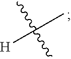 [SMILES: *[H]] |
| R² is | 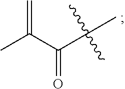 [SMILES: *C(=O)C(=C)C] |
| R³ is | 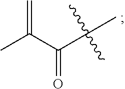 [SMILES: *C(=O)C(=C)C] |
| R⁴ is | 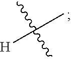 [SMILES: *[H]] |
| and | |
| R⁵ is | 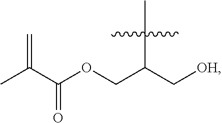 [SMILES: *C(CO)COC(=O)C(=C)C] |
| or where: | |
| R¹ is | 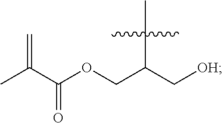 [SMILES: *C(CO)COC(=O)C(=C)C] |
| R² is | 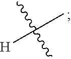 [SMILES: *[H]] |
| R³ is | 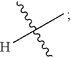 [SMILES: *[H]] |
| R⁴ is | 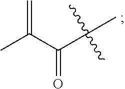 [SMILES: *C(=O)C(=C)C] |
| and | |
| R⁵ is | 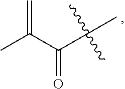 [SMILES: *C(=O)C(=C)C] |
| or where: | |
| R¹ is | 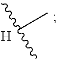 |
| R² is | 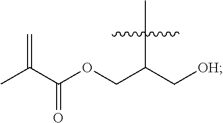 [SMILES: *C(CO)COC(=O)C(=C)C] |
| R³ is | 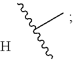 |
| R⁴ is | 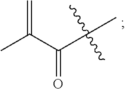 [SMILES: *C(=O)C(=C)C] |
| and | |
| R⁵ is | 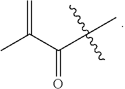 [SMILES: *C(=O)C(=C)C] |

Another aspect of this disclosure is a method of synthesizing a pH-responsive polymer. According to this aspect, the method may comprise: maintaining a vessel at a reaction temperature; mixing a gelatin with an acidic water in the vessel to form a first solution having a target pH; stirring the first solution at the reaction temperature while: adding an acid to the vessel to maintain the first solution at the target pH; beginning a reaction by adding glycidyl methacrylate to the first solution in the vessel in aliquots for a first time period to form a second solution; conducting the reaction in the absence of light for a second time period while maintaining the second solution at the target pH by obtaining a measured pH of the second solution at intervals during the second time period, determining a difference between the measured pH and the target pH at each one of the intervals, and adding additional acid to the second solution at each one of the intervals so that the measured pH equals the target pH; conducting the reaction in the absence of light for a third time period without maintaining the second solution at the target pH; adding additional acidic water to the second solution after the third time period; and removing unreacted glycidyl methacrylate from the second solution.

Maintaining the vessel at the reaction temperature may comprise placing the vessel in a temperature bath. The reaction temperature may be approximately 40° C. The reaction temperature may be between approximately 40° C. and approximately 50° C. The target pH may be 3.5. The gelatin may comprise Gelatin Type A. The method may comprise stirring the first solution at a rate of 800 rpm. The acidic water and the additional acidic water may have a pH of approximately 3.5. The acid may have a pH of approximately zero (0). The first time period may be approximately 2.5 hours. Beginning the reaction may comprise adding the glycidyl methacrylate to the first solution dropwise at a flowrate of approximately 700 μL/min in equal-volume aliquots at first intervals during the first time period. Each one of the first intervals may be approximately 30 minutes. The second time period may be approximately 12 hours. The target pH may be 3.0 or 3.5. Each one of the intervals may be approximately 30 minutes. The third time period may be approximately 12 hours.

Removing the unreacted glycidyl methacrylate may comprise: dialyzing the second solution; freezing the second solution; and lyophilizing the second solution. Dialyzing the second solution may comprise: transferring the second solution to a dialysis membrane; and removing the unreacted methacrylic anhydride by dialysis with the dialysis membrane. Removing the unreacted methacrylic anhydride with the dialysis membrane may comprise dialyzing the second solution against water, with the dialysis membrane, for a dialysis time period at a dialysis temperature. The dialysis temperature may be 40° C. The dialysis temperature may be approximately 40° C. The dialysis time period may be less than or equal to approximately 7 days. The dialysis time period may be at least approximately 5 days. The method may comprise adjusting the dialysis time period relative to a volume of the second solution. Freezing the second solution may comprise exposing the second solution to a freezing temperature equal to approximately −196° C. to create a frozen second solution. Lyophilizing the second solution may comprise placing the frozen second solution in a lyophilizer for a lyophilization time period. The method may comprise outputting a dry product of Polymer A from the lyophilizer. The lyophilization time period may be less than or equal to approximately 5 days. The lyophilization time period may be at least approximately 3 days. The method may comprise adjusting the lyophilization time period relative to a volume of the frozen second solution.

Another aspect of this disclosure is a bioink. According to this aspect, for example, the bioink may comprise: one or more of pH-responsive polymers; a photoinitiator; and a solvent. The one or more pH-responsive polymers may be selected from a group comprising: any variation of compound (I); any variation of compound (II); any variation of compound (III); and any variation of compound (IV). For example, the bioink may comprise: 10% w/v of GelMA; and 0.1% w/v of the compound of any variation of compound (I).

The one or more pH-responsive polymers may comprise at least two of: any variation of compound (I); any variation of compound (II); any variation of compound (III); and any variation of compound (IV). For example, the bioink may comprise 7% w/v of GelMA, 0.1% w/v of the compound of any variation of compound (I), and 0.05% w/v of the compound of any variation of compound (III); or 9% w/v of GelMA, 0.1% w/v of the compound of any variation of compound (I), and 0.05% w/v of the compound of any variation of compound (III); or 9% w/v of Polymer B, 0.2% w/v of the compound of any variation of compound (I), and 0.1% w/v of the compound of any variation of compound (III).

The one or more pH-responsive polymers may comprise at least three of: any variation of compound (I); any variation of compound (II); any variation of compound (III); and any variation of compound (IV). The one or more pH-responsive polymers may comprise: any variation of compound (I); any variation of compound (II); any variation of compound (III); and any variation of compound (IV). For example, the bioink may comprise 0.1% w/v of the compound of any variation of compound (I), 0.1% w/v of the compound of any variation of compound (II), 0.05% w/v of the compound of any variation of compound (III), and 12.0% w/v of the compound of any variation of compound (IV); or 0.1% w/v of the compound of any variation of compound (I), 0.1% w/v of the compound of any variation of compound (II), 0.1% w/v of the compound of any variation of compound (III), and 11.0% w/v of the compound of any variation of compound (IV); or 0.1% w/v of the compound of any variation of compound (I), 0.5% w/v of the compound of any variation of compound (II), 0.1% w/v of the compound of any variation of compound (III), and 15.0% w/v of the compound of any variation of compound (IV); or 0.1% w/v of the compound of any variation of compound (I), 0.5% w/v of the compound of any variation of compound (II), 0.5% w/v of the compound of any variation of compound (III), and 17.5% w/v of the compound of any variation of compound (IV); or 0.1% w/v of the compound of any variation of compound (I), 0.1% w/v of the compound of any variation of compound (II), 0.05% w/v of the compound of any variation of compound (III), and 7.0% w/v of the compound of any variation of compound (IV); or 0.1% w/v of the compound of any variation of compound (I), 0.5% w/v of the compound of any variation of compound (II), 0.5% w/v of the compound of any variation of compound (III), and 13.0% w/v of the compound of any variation of compound (IV).

The photoinitiator may be selected from a group comprising: lithium phenyl-2,4,6-trimethylbenzoylphosphinate (LAP); 2-hydroxy-4′-(2-hydroxyethoxy)-2-methylpropiophenone (Irgacure I2959); tetrapotassium-4,4′-(1,2-ethenediyl)bis[2-(3-sulfophenyl)diazenesulfonate] (AS7); E2CK; and P2CK. The solvent may comprise one of: phosphate buffered saline; a cell culture media; or water. Any bioink described herein may comprise GelMA, type A or type B.

Another aspect of this disclosure is a method of preparing bioinks. According to this aspect, the method may comprise: mixing one or more pH-responsive polymers into a mixture; forming a suspension by adding a photoinitiator solution to the mixture, the photoinitiator solution comprising a photoinitiator and a solvent; and mixing the suspension at a mixing rate and heating the suspension a temperature to fully dissolve components of the suspension.

The mixture may comprise a mixture of solids. The mixture may comprise a partially dissolved suspension of polymers in a phosphate buffered saline or a cell culture media. The photoinitiator solution may comprise a photoinitiator dissolved in the solvent. The solvent may comprise one of a phosphate buffered saline, a cell culture media, or water. The photoinitiator may be selected from a group comprising: lithium phenyl-2,4,6-trimethylbenzoylphosphinate (LAP); 2-hydroxy-4′-(2-hydroxyethoxy)-2-methylpropiophenone (Irgacure I2959); tetrapotassium-4,4′-(1,2-ethenediyl)bis[2-(3-sulfophenyl)diazenesulfonate](AS7); E2CK; and P2CK. Mixing the suspension may comprise stirring or vortex mixing. The method may comprise mixing the suspension at a first mixing rate of approximately 200 rpm to a form the mixture; and mixing the mixture a second mixing rate of between approximately 800 rpm and approximately 1500 rpm. The temperature may be at least 20° C. The temperature may be 50° C. The temperature may be between approximately 20° C. and approximately 50° C.

Another aspect of this disclosure is a method of determining characteristics of a bioink. According to this aspect, the method may comprise: preparing a hydrogel pellet of photo-crosslinked bioink; soaking the hydrogel pellet in a fluorescein sodium salt solution; drop-casting fluorescent polystyrene particles onto the surface of the hydrogel pellet; measuring elastic parameters of the hydrogel pellet; and calculating an elastic modulus of the hydrogel pellet.

The hydrogel pellet may comprise a diameter of 1 cm. The hydrogel pellet may comprise a thickness of approximately 300 to 700 μm. Preparing the hydrogel pellet may comprise: drop-casting a droplet of the bioink onto a PTFE surface; covering the droplet with a glass coverslip and spacer; and exposing the glass coverslip to a UV light for a first time period. The UV light may comprise 365 nm. The first time period may be approximately 3 minutes. Soaking the hydrogel pellet in the fluorescein sodium salt solution may comprise: transferring the hydrogel pellet and the cover slip to a well-plate; adding an amount of fluorescein sodium salt to the well-plate; adding an amount of phosphate buffered saline to the well-plate; and maintaining the well-plate at a temperature for a second time period.

The fluorescein sodium salt may comprise 35 μM fluorescein sodium salt in a phosphate buffered saline. The temperature may be approximately 25° C. The temperature may be between 20° C. and 30° C. The second time period may be approximately 16 hours. Drop-casting the fluorescent polystyrene particles onto the hydrogel pellet may comprise: transferring the hydrogel pellet and coverslip to a microscope sample holder; drop-casting the fluorescent polystyrene particles onto the hydrogel pellet; and allowing the fluorescent polystyrene particles to settle for a third time period. The fluorescent polystyrene particles may be suspended in water. The third time period may be at least 5 minutes. Measuring the elastic parameters of the hydrogel pellet may comprise: placing an indenter onto the hydrogel pellet; measuring a depth of an indention made on the hydrogel pellet; and measuring a thickness of the hydrogel pellet. A first ratio of a radius of the indenter to the thickness of the hydrogel pellet is between 0.3 and 12. A second ratio of the depth of indentation to the thickness of the hydrogel pellet is less than 0.6.

The method may comprise using laser scanning confocal microscopy to measure one or both of: the depth of the indention; and the thickness of the hydrogel pellet. Calculating the elastic modulus may comprise using a modified Hertz model based on variables comprising: the elastic parameters of the hydrogel pellet; a force applied by the indenter; acceleration due to gravity; a density of the indenter; and a density of a medium. The medium may comprise a phosphate buffered saline.

### BRIEF DESCRIPTION OF THE DRAWINGS

The accompanying drawings, which are incorporated in and constitute part of this disclosure, illustrate exemplary aspects that, together with the written descriptions, explain the principles of this disclosure. Numerous aspects are particularly described, pointed out, and taught in the written descriptions. Some aspects may be even better understood by referencing the written portions together with the accompanying drawings, of which:

FIG. 1 shows an exemplary reaction for synthesizing methacrylated collagen.

FIG. 2 shows an exemplary method of synthesizing the FIG. 1 collagen.

FIG. 3 shows exemplary method steps for synthesizing the FIG. 1 collagen.

FIG. 3A shows exemplary method steps for synthesizing the FIG. 1 collagen.

FIG. 4 shows an exemplary ¹H NMR of the FIG. 1 collagen deuterated water.

FIG. 5 shows an exemplary reaction for synthesizing methacrylated hydrolyzed elastin.

FIG. 6 shows an exemplary method of synthesizing the FIG. 5 elastin.

FIG. 7 shows exemplary method steps for synthesizing the FIG. 5 elastin.

FIG. 8 shows an exemplary ¹H NMR of the FIG. 5 elastin in deuterated water.

FIG. 9 shows an exemplary reaction for synthesizing methacrylated hyaluronic acid.

FIG. 10 shows an exemplary method of synthesizing the FIG. 9 acid.

FIG. 11 shows exemplary method steps for synthesizing the FIG. 9 acid.

FIG. 11A shows exemplary method steps for synthesizing the FIG. 9 acid.

FIG. 11B shows exemplary method steps for synthesizing the FIG. 9 acid.

FIG. 12 shows an exemplary ¹H NMR of the FIG. 9 acid in deuterated water.

FIG. 13 shows an exemplary reaction for synthesizing Polymer A.

FIG. 14 shows an exemplary method of synthesizing the FIG. 13 polymer.

FIG. 15 shows exemplary method steps for synthesizing the FIG. 13 polymer.

FIG. 16 shows an exemplary ¹H NMR of the FIG. 13 polymer in deuterated water.

FIG. 17 shows exemplary structures of one-photon photoinitiators.

FIG. 18 shows exemplary structures of two-photon photoinitiators.

FIG. 19 shows an exemplary table of bioink compositions.

FIG. 20 shows an exemplary method of preparing the FIG. 19 bioink compositions.

FIG. 21 shows exemplary method steps for the FIG. 20 method.

FIG. 22 shows an exemplary table of elastic moduli for soft tissues and supporting citations.

FIG. 23 shows an exemplary method of determining characteristics of a bioink by forming a hydrogel pellet from the bioink.

FIG. 24 shows exemplary method steps for the FIG. 23 method.

FIG. 25 shows exemplary laser scanning confocal microscopy images of the FIG. 23 pellet.

FIG. 26 shows an exemplary method for calculating an elastic modulus of the FIG. 23 pellet.

FIG. 27 shows an exemplary table of measured elastic moduli for bioink hydrogels.

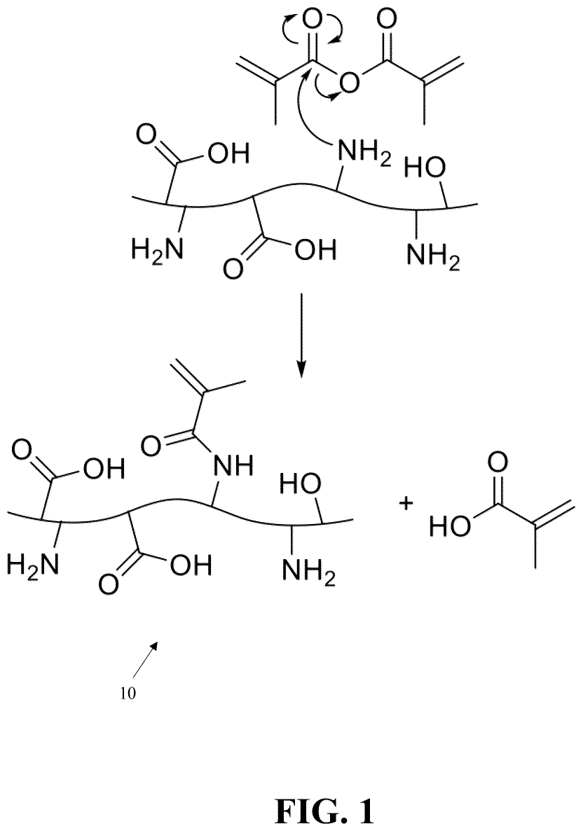

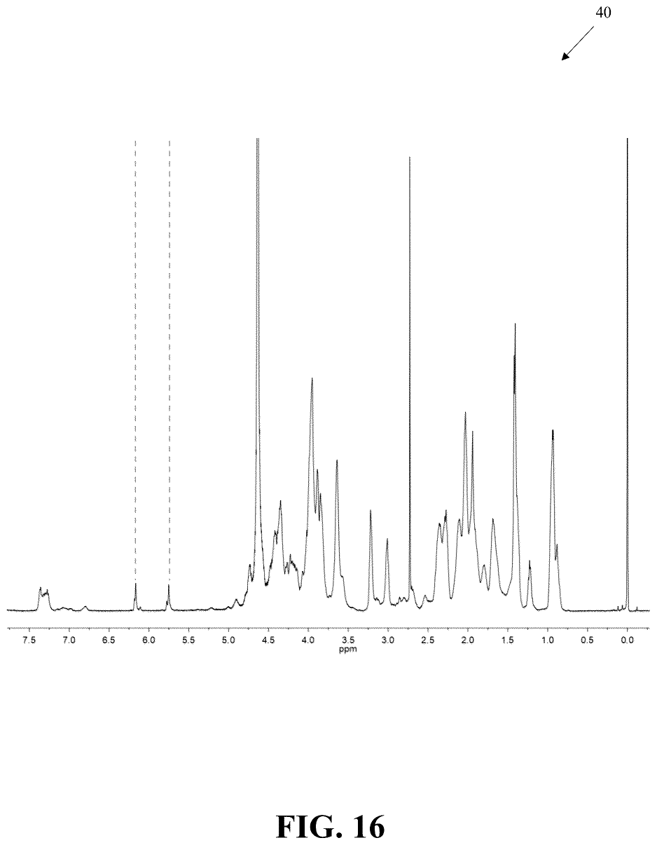

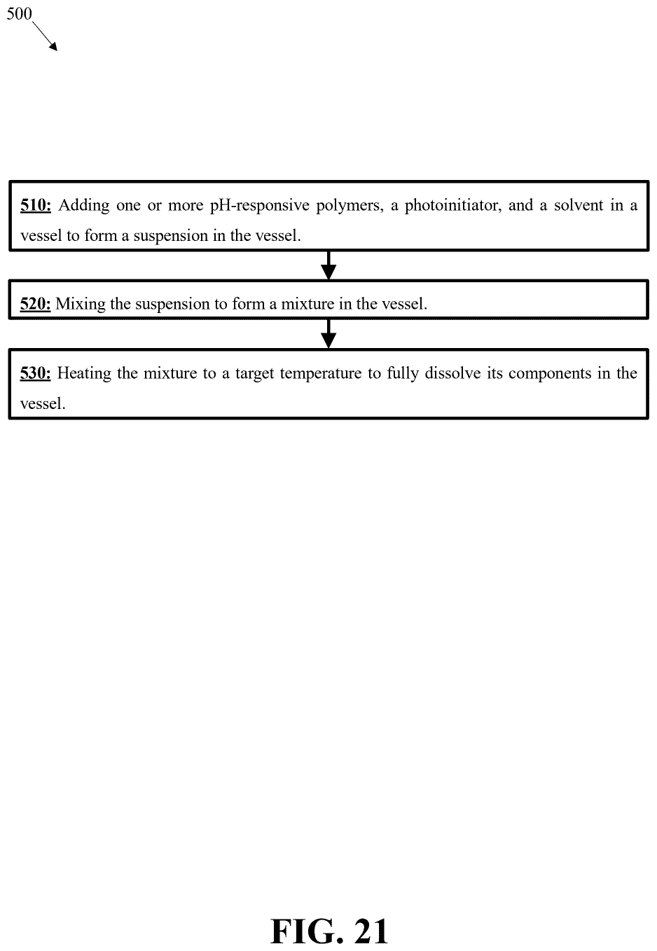

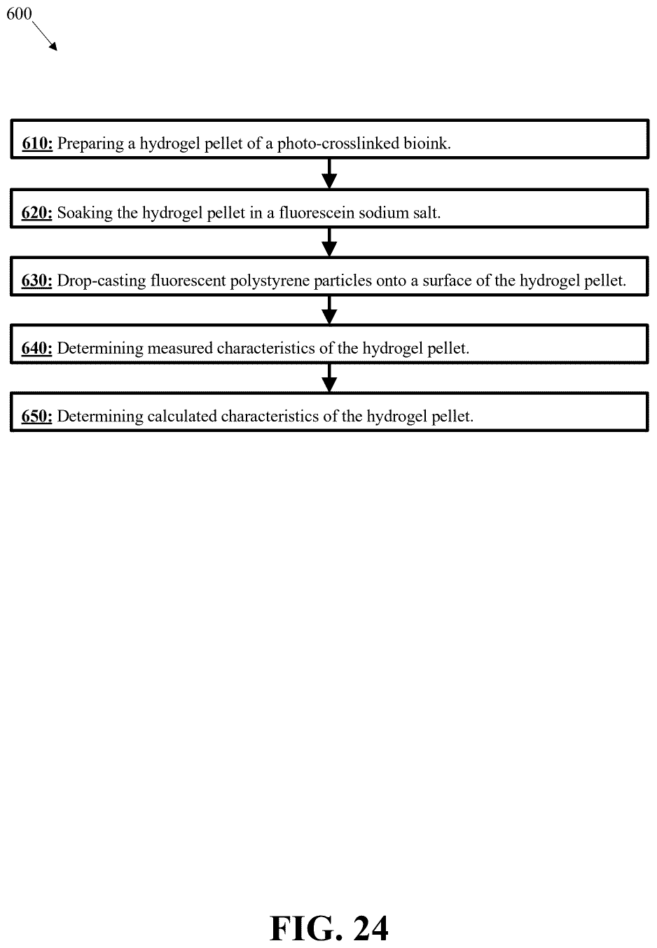

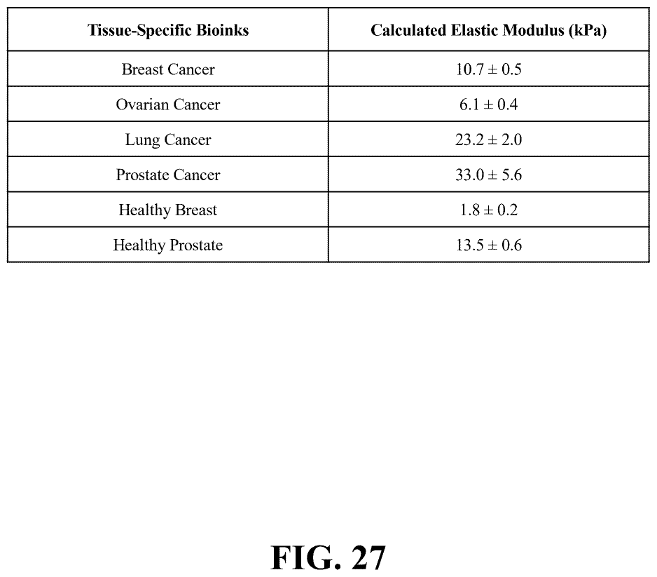

Aspects described or depicted with respect to one or more FIGS. 1 to 27 may be incorporated in different aspects although not specifically described relative thereto. Aspects described herein and/or features thereof may be combined in any way and/or combination. These and other aspects of the present disclosure are explained in detail in the written descriptions contained herein. Further features, advantages, and details of aspects of this disclosure may be appreciated by reading FIGS. 1 to 27 together with the accompanying written descriptions.

### DETAILED DESCRIPTION

Aspects of the present disclosure are not limited to the examples described in the written descriptions and shown in the accompanying drawings. Many aspects of this disclosure may be applicable to other aspects and/or capable of being practiced or carried out in various variants of use, including the examples described herein and shown in the drawings.

Throughout this disclosure, specific details are set forth with particularity to provide a more thorough understanding to persons of ordinary skill in the art. For convenience and ease of description, some well-known aspects may be described conceptually to avoid unnecessarily obscuring the focus of this disclosure. In this regard, the written descriptions and accompanying drawings should be interpreted as illustrative rather than restrictive, enabling rather than limiting.

Exemplary aspects of this disclosure reference bioinks and methods of synthesizing, characterizing, and deploying bioinks. Some aspects are described with reference to particular elements of bioinks, such as one or more pH-responsive polymers (e.g., such as a methacrylated collagen, a methacrylated hydrolyzed elastin, a methacrylated hyaluronic acid, a methacrylated gelatin, and/or the like), made with a particular methods (e.g., by controlling the pH of a synthesis reaction), to realize particular benefits (e.g., predictable swelling characteristics relative to the pH of a medium, customizability for mimicking different tissues, interoperability with different 3D bioprinters, and/or repeatable manufacturing processes). Unless claimed, these descriptions are provided for convenience and not intended to limit the present disclosure unless recited in the claims set forth below.

Terms such as “may,” “can,” and like variations, are intended to describe optional aspects of this disclosure, any of which may be covered by the claims set forth below. Inclusive terms such as “comprises,” “comprising,” “includes,” “including,” and variations thereof, are intended to cover a non-exclusive inclusion, such that an apparatus, composition, method, system, or element thereof comprising a list of elements does not include only those elements but may include other elements not expressly listed and/or inherent thereto.

The term “exemplary” is used in the sense of “example,” rather than “ideal.” The term “and/or” indicates a potential combination, such that a first and/or second element may likewise be described as a first element, a second element, or a combination thereof, each of which being an example. Numerous other combinations are inherent to this disclosure. Various terms of approximation may be used, including “approximately” and “generally.” Approximately means within 10% of and generally means “within most cases” or “usually.”

The term “biocompatible” is intended to describe any material that does not elicit a substantial detrimental response in vivo. The term “R Groups” is intended to describe any combination of R¹, R², R³, R⁴, and R⁵ as those terms are defined below. The term “selectively” is intended to describe an intentional change, or not change, using specific materials or altering the parameters or properties including molecules, compounds, polymers, tools, temperature, pH, time, and/or speed.

The term “solution” is intended to describe any liquid mixture comprising two or more components. Some described solutions may not be truly homogenous and completely free of extraneous materials. The term “mixture” is intended to describe a combination of two more components that may include soluble and/or insoluble ingredients. The term composition is intended to describe an aggregate, mixture, mass, or body formed by combining two or more elements or ingredients. Terms like composition and mixture may be synonymous in that any composition described herein may exist at the moment its ingredients are mixed together.

Terms such as “bioink,” “tissue-specific bioink,” and “universal bioink” may be used in this application. The term “bioink” may generically describe any bioink produced according to this disclosure for use with a 3D bioprinter operable to mimic an extracellular matrix therewith by forming hydrogels through a crosslinking process. The term “tissue-specific bioink” may describe a particular type of bioink produced according to this disclosure for use with a 3D bioprinter operable to mimic a particular tissue type therewith. The term “universal bioink” may describe a particular type of bioink that has been produced according to this disclosure for use with a particular 3D bioprinting technology and/or crosslinking method operable therewith, allowing tissue models, scaffolds, and/or constructs for mimicking human tissue to be produced with different 3D bioprinting methods, including extrusion-based 3D bioprinting, digital light processing (DLP) 3D bioprinting, and 3D bioprinting with two-photon polymerization. Some bioinks described herein may be “tissue-specific” and “universal,” meaning that their tissue-specific aspects may be consistently realized with different 3D bioprinting technologies.

Aspects of different pH-responsive polymers and methods of synthesizing them are now described in detail, followed by detailed descriptions of bioinks comprising one or more of the different pH-responsive polymers, tissue-specific bioinks comprising one or more of the different pH-responsive polymers, universal bioinks comprising one or more of the different pH-responsive polymers, methods of synthesizing different bioinks, methods of determining characteristics of different bioinks, and benefits associated with the different bioinks described herein.

### Methacrylated Collagen and Methods of Synthesizing the Same

Now described with reference to FIGS. 1 to 4 is a first pH-responsive polymer or “methacrylated collagen.” As shown in FIGS. 1 and/or 2, for example, a methacrylated collagen 10 may comprise a synthesized polymer prepared by controlling the pH of a synthesis reaction with Type 1 rat tail collagen, resulting in a first type of pH-responsive polymer that has been selectively modified and further processed into a first dry product according to this disclosure.

Methacrylated collagen 10 may be a pH-responsive polymer comprising Type 1 rat tail collagen with selectively modified and unmodified functional groups. As shown in FIG. 1, for example, methacrylated collagen 10 may comprise a compound of formula (I), wherein: (A) each R Group may attach to an amine group that has been reacted though an acyl substitution mechanism, allowing a methacryloyl group to attach; and (B) each amine group may not have reacted though an acyl substitution mechanism, allowing the amine group to remain as its respective functional group.

As shown in FIG. 1, for example, methacrylated collagen 10 may comprise a compound of formula (I) comprising:

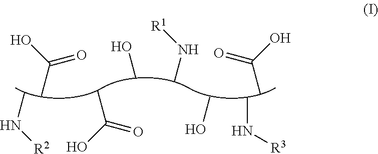

wherein:
the compound comprises a collagen backbone.
R¹, R², and R³ are selected from a group consisting of:

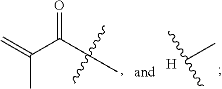

at least one of R¹, R², and R³ is not

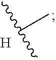

and
“ [symbol] ” indicates the point of attachment.

As shown in FIG. 1, for example, methacrylated collagen 10 may comprise a compound of formula (I) comprising: (A) amine groups that have been methacrylated; or (B) carboxyl, hydroxyl, and amine groups that were not methacrylated.

The methacrylated and unmethacrylated functional groups of methacrylated collagen may be selected by controlling the pH level of the synthesizing reaction. As shown in FIGS. 1 and/or 2, for example, by maintaining pH levels during the synthesizing reaction: (A) the modified functional groups of methacrylated collagen 10 may comprise methacrylated amine groups and (B) the unmodified functional groups of methacrylated collagen may comprise (i) unreacted amine groups, (ii) unreacted carboxyl groups, or (iii) unreacted hydroxyl groups.

To provide additional examples of methacrylated collagen 10, the compound of formula (I) also may comprise

| where: | |
|---|---|
| R¹ is | 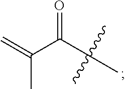 [SMILES: *C(=O)C(=C)C] |
| R² is | 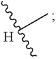 |
| and | |
| R³ is | 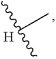 |
| or where: | |
| R¹ is | 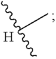 |
| R² is | 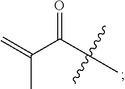 [SMILES: *C(=O)C(=C)C] |
| and | |
| R³ is | 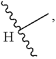 |
| or where: | |
| R¹ is | 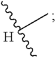 |
| R² is | 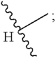 |
| and | |
| R³ is | 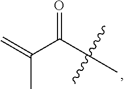 [SMILES: *C(=O)C(=C)C] |
| or where: | |
| R¹ is | 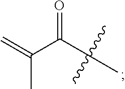 [SMILES: *C(=O)C(=C)C] |
| R² is | 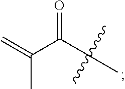 [SMILES: *C(=O)C(=C)C] |
| and | |
| R³ is | 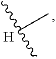 |
| or where: | |
| R¹ is | 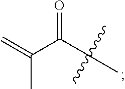 [SMILES: *C(=O)C(=C)C] |
| R² is | 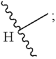 |
| and | |
| R³ is | 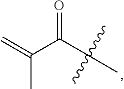 [SMILES: *C(=O)C(=C)C] |
| or where: | |
| R¹ is | 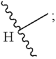 |
| R² is | 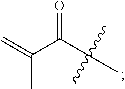 [SMILES: *C(=O)C(=C)C] |
| and | |
| R³ is | 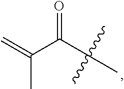 [SMILES: *C(=O)C(=C)C] |
| or where: | |
| R¹ is | 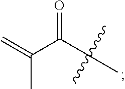 [SMILES: *C(=O)C(=C)C] |
| R² is | 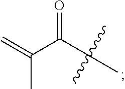 [SMILES: *C(=O)C(=C)C] |
| and | |
| R³ is | 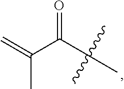 [SMILES: *C(=O)C(=C)C] |

each being examples of methacrylated collagen 10 according to this disclosure.

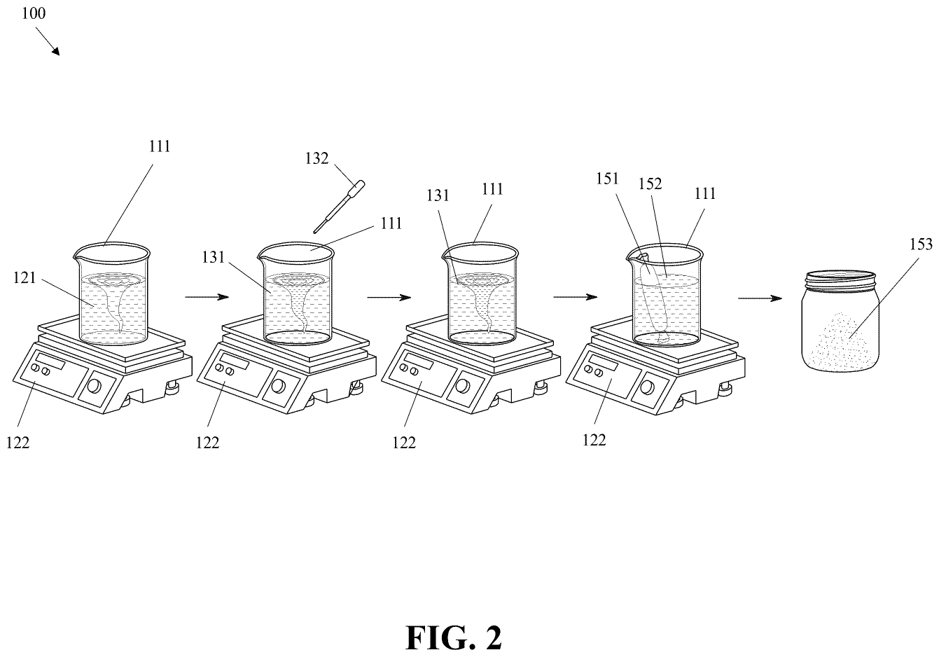

Aspects of an exemplary method 100 of synthesizing methacrylated collagen 10 are now described with continued reference to FIGS. 1 to 4. Method 100 may comprise modifying Type 1 rat tail collagen by methacrylating its functional groups. As shown in FIGS. 1, 2, and/or 3, for example, method 100 may comprise synthesizing methacrylated collagen by methacrylating the Type 1 rat tail collagen with methacrylic anhydride or an equivalent thereof. As further shown in FIGS. 1, 2, and/or 3, for example, method 100 may comprise reacting the Type 1 rat tail collagen with methacrylic anhydride and maintaining a basic pH of the resulting solution to achieve selective modification of its functional groups.

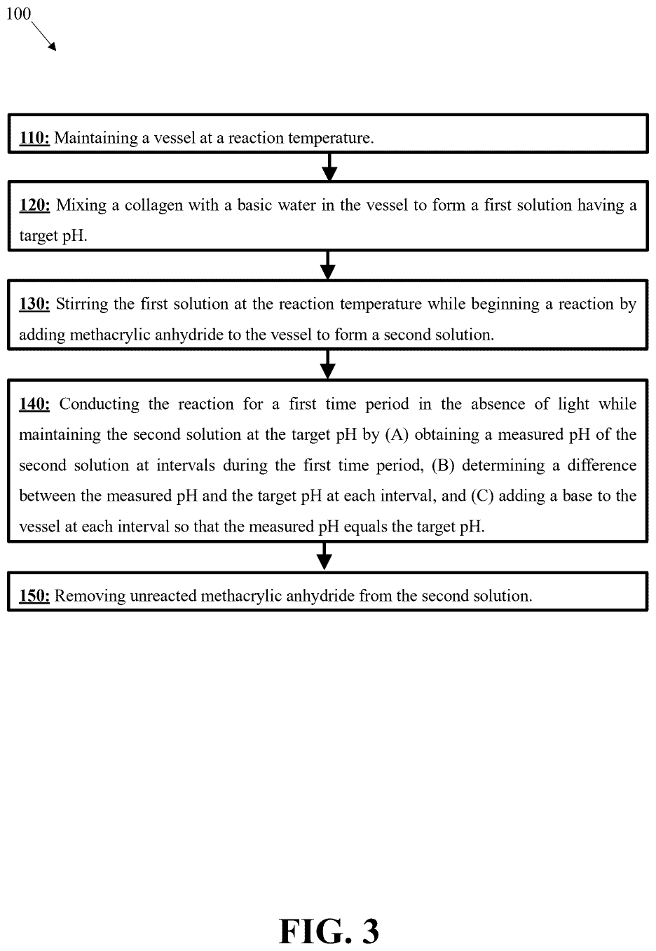

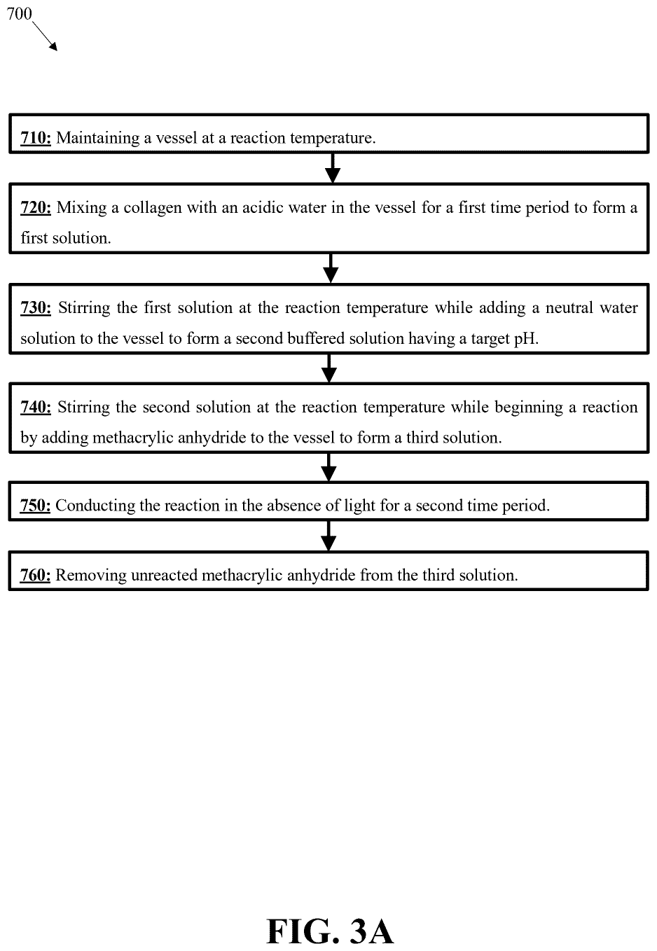

As shown in FIG. 3, for example, method 100 may comprise: (I) maintaining a vessel at a reaction temperature (a maintaining step 110); (II) mixing a collagen with a basic water in the vessel to form a first solution having a target pH (a mixing step 120); (III) stirring the first solution at the reaction temperature while beginning a reaction by adding methacrylic anhydride to the vessel to form a second solution (a reaction step 130), conducting the reaction for a first time period in the absence of light while maintaining the second solution at the target pH by (A) obtaining a measured pH of the second solution at intervals during the first time period, (B) determining a difference between the measured pH and the target pH at each one of the intervals, and (C) adding a base to the vessel at each one of the intervals so that the measured pH equals the target pH (a maintaining step 140); and (IV) removing unreacted methacrylic anhydride from the second solution (a removing step 150).

As shown in FIGS. 2 and/or 3, for example, maintaining step 110 may comprise maintaining a temperature bath (e.g., an electrothermal thermostatic water bath, not shown) at the reaction temperature and placing a vessel 111 in the temperature bath. The reaction temperature may be automatically maintained by a temperature control system of the temperature bath. For step 110, the reaction temperature may be room temperature, such as approximately 20° C., or between approximately 18° C. and approximately 25° C., or between 18° C. and 25° C.

As shown in FIGS. 2 and/or 3, for example, mixing step 120 may comprise forming a first solution 121 in vessel 111 by adding an amount of type 1 rat tail collagen to vessel 111 and mixing it with a reactant in vessel 111. For example, mixing step 120 may comprise forming first solution 121 by dissolving 25 mg of type 1 rat tail collagen in 20 mM acetic acid in vessel 111 at a concentration of 5 mg/mL; adding 2 M sodium hydroxide to vessel 111 at a flow rate of approximately 1 mL/min until the target pH reaches 10; and stirring the contents of vessel 111 at a stir rate (e.g., such as approximately 600 rpm) with a hotplate magnetic stirrer 122. While vessel 111 is maintained at the reaction temperature and its contents are being stirred at the stir rate with hotplate magnetic stirrer 122, reaction step 130 may comprise adding methacrylic anhydride to vessel 111 at a flowrate of approximately 100 μL/min to form a second solution 131 in vessel 111. As shown in FIG. 2, for example, the methacrylic anhydride may be added dropwise with a dropper 132.

As shown in FIGS. 2 and/or 3, for example, reaction step 140 may comprise wrapping vessel 111 in a light blocking material, such as aluminum foil, and/or performing step 140 in a light-free environment, such as a dark room. As further shown, reaction step 140 may comprise obtaining the measured pH of second solution 131 with a pH sensor and comparing it to the target pH of second solution 131 at regular intervals during the first time period. As shown in FIGS. 2 and/or 3, for example, reaction step 140 also may comprise adding the base, such as 2 M sodium hydroxide, to second solution 131 at each interval so that its measured pH equals its target pH of 10 during the first time period. For example, the intervals may comprise 10-minute intervals and the first time period may be conducted for approximately 2 hours.

As shown in FIGS. 2 and/or 3, for example, removing step 150 may comprise transferring second solution 131 to dialysis membranes 151 and removing unreacted methacrylic anhydride from second solution 131 by dialysis with dialysis membranes 151. Dialysis may be performed against a volume of water 152 at a dialysis temperature (e.g., such as approximately 30° C.) for a dialysis time period of between at least 5 days and approximately 7 days, adjustable relative to the volume of second solution 131. As shown in FIGS. 2 and/or 3, for example, removing step 150 may further comprise freezing second solution 131 to a freezing temperature (e.g., such as approximately −196° C.) and lyophilizing the frozen second solution 131 for a lyophilization time period to obtain a dry product pH-responsive polymer 153. For example, the lyophilization time period may be between at least 3 days and approximately 7 days, adjustable relative to the volume of solution 131.

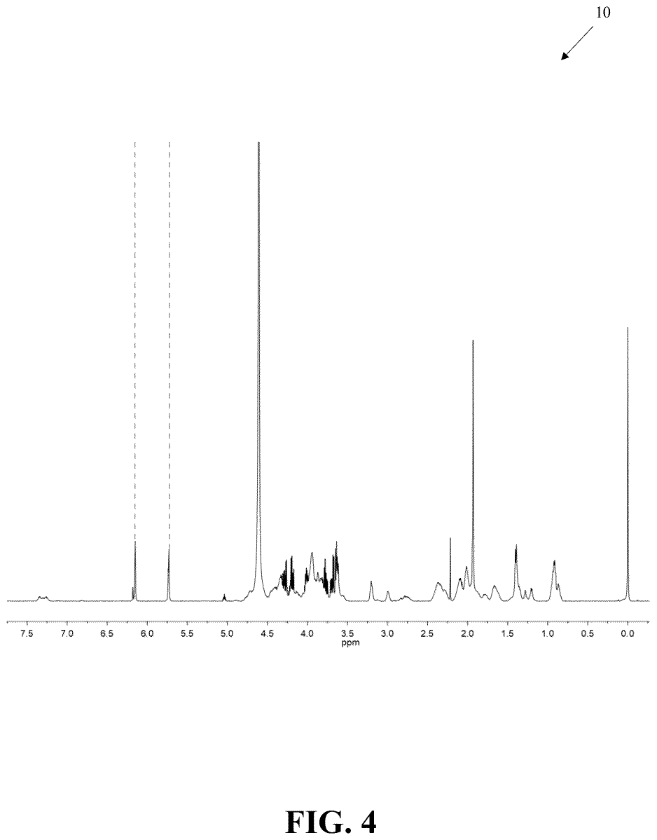

As shown in FIG. 4, for example, the formation of methacrylated collagen 10 may be verified by ¹H NMR spectroscopy.

Alternative methods of synthesizing methacrylated collagen 10 are contemplated. By way of example, aspects of an exemplary method 700 of synthesizing methacrylated collagen 10 are now described with continued reference to FIGS. 1, 2, 3A, and/or 4. As shown in FIG. 3A, for example, method 700 may comprise (I) maintaining a vessel (e.g., like vessel 111 of FIG. 2) at a reaction temperature (a maintaining step 710); (II) mixing a collagen with an acidic water in the vessel for a first time period to form a first solution (a mixing step 720); (III) stirring the first solution at the reaction temperature while adding a neutral water to the vessel to form a second buffered solution having a target pH (a forming step 730); (IV) stirring the second solution at the reaction temperature while beginning a reaction by adding methacrylic anhydride to the vessel to form a third solution (a forming step 740); (V) conducting the reaction in the absence of light for a second time period (a reaction step 750); and (VI) removing unreacted methacrylic anhydride from the third solution (a removing step 760).

As shown in FIGS. 2 and/or 3A, for example, maintaining step 710 may comprise maintaining a temperature bath (e.g. an ice/water bath, or an electrothermal cryogenic bath, not shown) or a temperature-controlled environment (e.g. a refrigerator or cold room, also not shown) and placing the vessel in the temperature bath or the temperature-controlled environment. The reaction temperature of the vessel may be manually or automatically maintained by a temperature control system of the temperature bath or the temperature-controlled environment. For step 710, the reaction temperature may be lower than in step 110, such as approximately 5° C., or between approximately 3° C. and approximately 6° C., or between 3° C. and 6° C.

As shown in FIGS. 2 and/or 3A, for example, mixing step 720 may comprise forming a first solution in the vessel by adding an amount of type 1 rat tail collagen to the vessel and mixing it with a solvent in the vessel. For example, mixing step 720 may comprise forming the first solution by dissolving 100 mg of type 1 rat tail collagen in 10 mM aqueous hydrochloric acid at a concentration of 4 mg/mL and mixing the first solution at a stir rate (e.g. such as approximately 600 rpm) with a magnetic stirrer (e.g., like stirrer 122 of FIG. 2). The first time period may be approximately 24 hours.

As shown in FIGS. 2 and/or 3A, for example, forming step 730 may comprise forming the second buffered solution by maintaining the first solution at the reaction temperature and stirring it at the stir rate (e.g. such as approximately 600 rpm) while adding 0.2M sodium hydrogen phosphate dibasic in 500 μL aliquots until the second buffered solution reaches a target pH of 7.5.

As shown in FIGS. 2 and/or 3A, for example, forming step 740 may comprise forming the third solution by maintaining the second buffered solution at the reaction temperature and stirring it at the stir rate (e.g. such as approximately 600 rpm) while adding 20 μL of methacrylic anhydride to the vessel.

As shown in FIGS. 2 and/or 3A, for example, reaction step 750 may comprise wrapping the vessel in a light blocking material, such as aluminum foil, and/or performing step 750 in a light-free environment, such as a dark room. In step 750, the second time period may be approximately 8 hours.

As shown in FIGS. 2 and/or 3A, for example, removing step 760 may comprise transferring the third solution to dialysis membranes (e.g., like membranes 151 of FIG. 2) and removing unreacted methacrylic anhydride by dialysis with the dialysis membranes. Dialysis may be performed against a volume of 10 mM HCl at a dialysis temperature (e.g. such as approximately 25° C., or approximately 4° C., or between 4° C. and 25° C.) for a dialysis time period of between two and 3 days. As shown in FIGS. 2 and/or 3A, for example, removing step 760 further comprise freezing the third solution to a freezing temperature (e.g. such as approximately −196° C.) and lyophilizing the frozen third solution for a lyophilization time period to obtain a dry product pH-responsive polymer 153. For example, the lyophilization time period may be between at least 3 days and approximately 7 days, adjustable to the relative volume of the third solution.

In keeping with above, as shown in FIG. 4, for example, the formation of methacrylated collagen 10 may be verified by ¹H NMR spectroscopy.

### Methacrylated Hydrolyzed Elastin and Methods of Synthesizing the Same

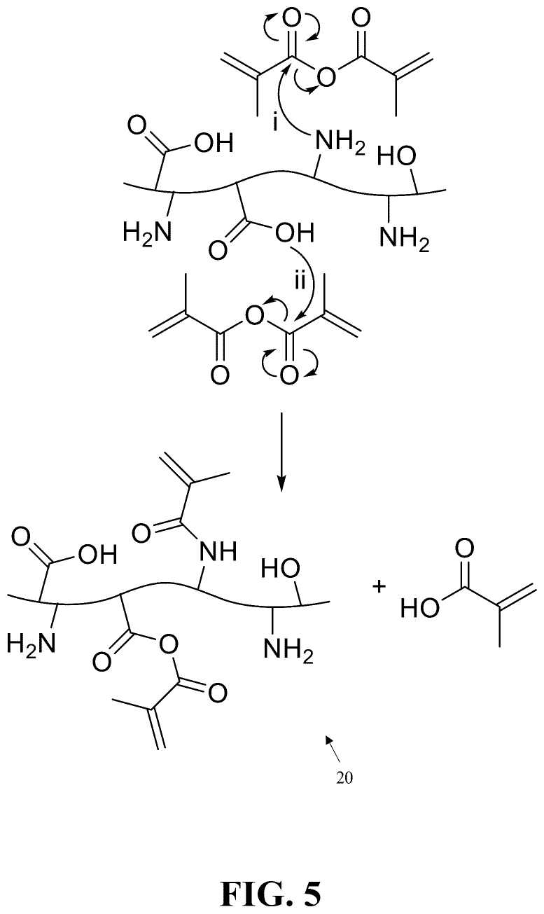

Now described with reference to FIGS. 5 to 8 is a second pH-responsive polymer or “methacrylated hydrolyzed elastin.” As shown in FIGS. 5 and/or 6, for example, a methacrylated hydrolyzed elastin 20 may comprise a synthesized polymer prepared by controlling the pH of a synthesis reaction with a hydrolyzed elastin, resulting in a second type of pH-responsive polymer that has been selectively modified and further processed into a second dry product according to this disclosure.

Methacrylated hydrolyzed elastin 20 may be a pH-responsive polymer comprising a hydrolyzed elastin with selectively modified and unmodified functional groups. As shown in FIG. 5, for example, methacrylated hydrolyzed elastin 20 may comprise a compound of formula (II), wherein: (A) each R Group may attach to a carboxyl group or a hydroxyl group that has been reacted though an epoxide ring-opening mechanism, allowing a methacrylate group to attach; (B) each carboxyl or hydroxyl group may not have reacted though an epoxide ring-opening mechanism, allowing the carboxyl or hydroxyl group to remain as its respective functional group; and (C) the amine group may be reacted through an epoxide ring-opening mechanism, allowing a methacrylate group to attach.

As shown in FIG. 5, for example, methacrylated hydrolyzed elastin 20 may comprise a compound of formula (II) comprising:

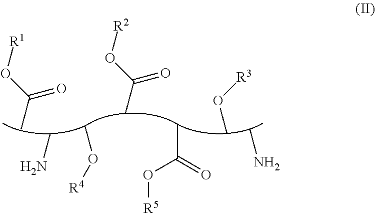

wherein:
the compound comprises an elastin backbone;
R¹, R², R³, R⁴, and R⁵ are selected from a group consisting of:

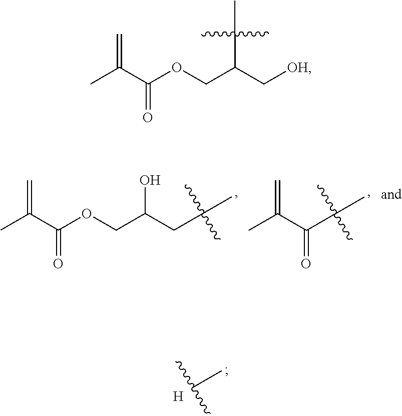

at least one of R¹, R², R³, R⁴, and R⁵ is not

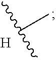

and
“ [symbol] ” indicates the point of attachment.

As shown in FIG. 5, for example, second pH-responsive polymer or methacrylated hydrolyzed elastin 20 may comprise a compound of formula (II) comprising: (A) modified carboxyl groups, modified hydroxyl groups, unmodified carboxyl groups, unmodified hydroxyl groups, and unmodified amine groups; (B) carboxyl and hydroxyl groups that have been methacrylated; or (C) carboxyl, hydroxyl, and amine groups that were not methacrylated.

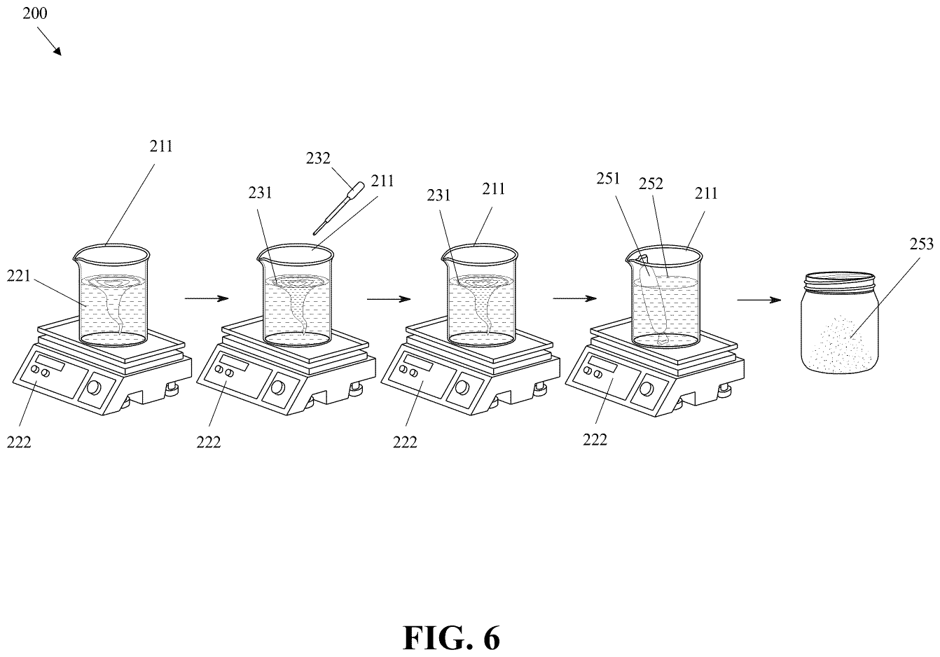

The methacrylated and modified and unmodified functional groups of methacrylated hydrolyzed elastin 20 may be selected by controlling the pH level of the synthesizing reaction. As shown in FIGS. 5, 6, and/or 7, for example, by maintaining pH levels during the synthesizing reaction: (A) the modified functional groups of methacrylated hydrolyzed elastin 20 may consist of (i) methacrylated carboxyl groups and methacrylated hydroxyl groups or (ii) methacrylated amine groups and methacrylated hydroxyl groups; (B) the unmodified functional groups of methacrylated hydrolyzed elastin 20 may consist of (i) unreacted amine groups, (ii) unreacted carboxyl groups, or (iii) unreacted hydroxyl groups.

To provide additional examples for methacrylated hydrolyzed elastin 20, the compound of formula (II) also may comprise

| where: | |
|---|---|
| R¹ is | 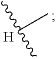 |
| R² is | 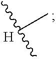 |
| R³ is | 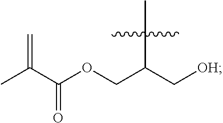 [SMILES: *C(CO)COC(=O)C(=C)C] |
| R⁴ is | 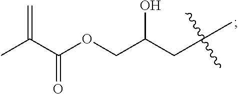 [SMILES: *CC(O)COC(=O)C(=C)C] |
| and | |
| R⁵ is | 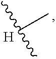 |
| or where: | |
| R¹ is | 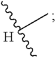 |
| R² is | 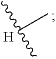 |
| R³ is | 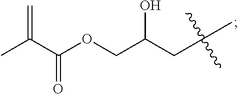 [SMILES: *CC(O)COC(=O)C(=C)C] |
| R⁴ is | 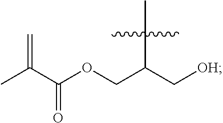 [SMILES: *C(CO)COC(=O)C(=C)C] |
| and | |
| R⁵ is | 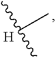 |
| or where: | |
| R¹ is | 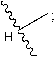 |
| R² is | 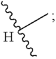 |
| R³ is | 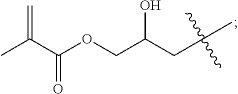 [SMILES: *CC(O)COC(=O)C(=C)C] |
| R⁴ is | 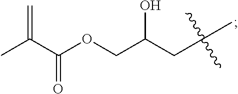 [SMILES: *CC(O)COC(=O)C(=C)C] |
| and | |
| R⁵ is | 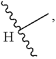 |
| or where: | |
| R¹ is | 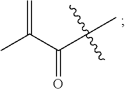 [SMILES: *C(=O)C(=C)C] |
| R² is | 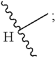 |
| R³ is | 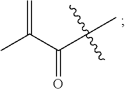 [SMILES: *C(=O)C(=C)C] |
| R⁴ is | 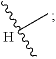 |
| and | |
| R⁵ is | 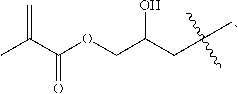 [SMILES: *CC(O)COC(=O)C(=C)C] |
| or where: | |
| R¹ is | 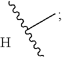 |
| R² is | 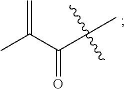 [SMILES: *C(=O)C(=C)C] |
| R³ is | 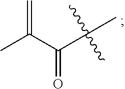 [SMILES: *C(=O)C(=C)C] |
| R⁴ is | 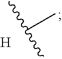 |
| and | |
| R⁵ is | 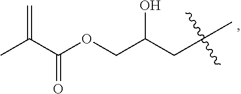 [SMILES: *CC(O)COC(=O)C(=C)C] |
| or where: | |
| R¹ is | 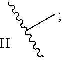 |
| R² is | 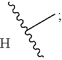 |
| R³ is | 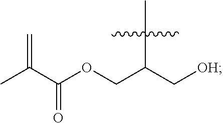 [SMILES: *C(CO)COC(=O)C(=C)C] |
| R⁴ is | 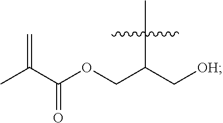 [SMILES: *C(CO)COC(=O)C(=C)C] |
| and | |
| R⁵ is | 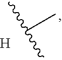 |
| or where: | |
| R¹ is | 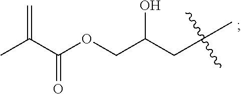 [SMILES: *CC(O)COC(=O)C(=C)C] |
| R² is | 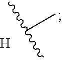 |
| R³ is | 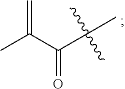 [SMILES: *C(=O)C(=C)C] |
| R⁴ is | 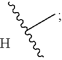 |
| and | |
| R⁵ is | 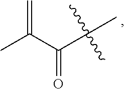 [SMILES: *C(=O)C(=C)C] |
| or where: | |
| R¹ is | 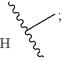 |
| R² is | 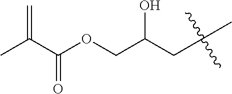 [SMILES: *CC(O)COC(=O)C(=C)C] |
| R³ is | 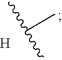 |
| R⁴ is | 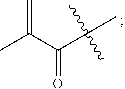 [SMILES: *C(=O)C(=C)C] |
| and | |
| R⁵ is | 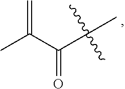 [SMILES: *C(=O)C(=C)C] |
| or where: | |
| R¹ is | 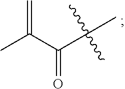 [SMILES: *C(=O)C(=C)C] |
| R² is | 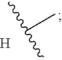 |
| R³ is | 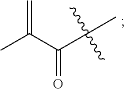 [SMILES: *C(=O)C(=C)C] |
| R⁴ is | 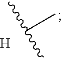 |
| and | |
| R⁵ is | 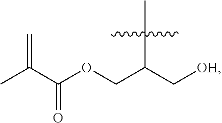 [SMILES: *C(CO)COC(=O)C(=C)C] |
| or where: | |
| R¹ is | 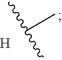 |
| R² is | 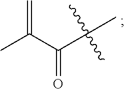 [SMILES: *C(=O)C(=C)C] |
| R³ is | 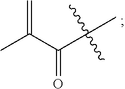 [SMILES: *C(=O)C(=C)C] |
| R⁴ is | 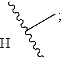 |
| and | |
| R⁵ is | 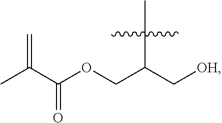 [SMILES: *C(CO)COC(=O)C(=C)C] |
| or where: | |
| R¹ is | 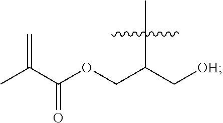 [SMILES: *C(CO)COC(=O)C(=C)C] |
| R² is | 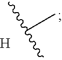 |
| R³ is | 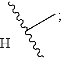 |
| R⁴ is | 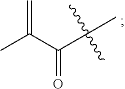 [SMILES: *C(=O)C(=C)C] |
| and | |
| R⁵ is | 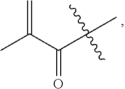 [SMILES: *C(=O)C(=C)C] |
| or where: | |
| R¹ is | 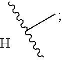 |
| R² is | 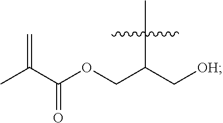 [SMILES: *C(CO)COC(=O)C(=C)C] |
| R³ is | 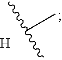 |
| R⁴ is | 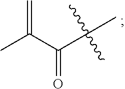 [SMILES: *C(=O)C(=C)C] |
| and | |
| R⁵ is | 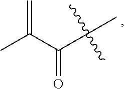 [SMILES: *C(=O)C(=C)C] |

each of the above being examples of methacrylated hydrolyzed elastin 20 according to this disclosure.

Method 200 may comprise a first method 201 of synthesizing a hydrolyzed elastin and a second method 209 of synthesizing methacrylated hydrolyzed elastin 20.

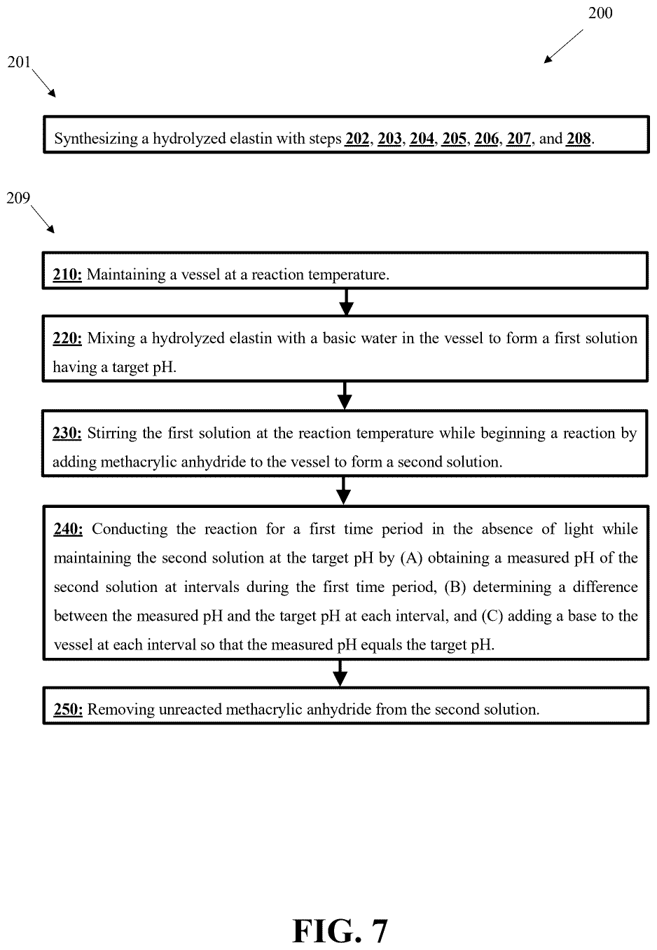

As shown in FIG. 7, for example, method 201 may comprise (I) suspending an amount of elastin in an aqueous oxalic acid solution to form a suspension (a suspending step 202); (II) refluxing the suspension for an initial time period at an initial temperature (a refluxing step 203); (III) cooling the suspension to a cooled temperature after the initial time period (a cooling step 204) (IV); centrifuging the suspension for a subsequent time period at the cooled temperature (a centrifuging step 205); (V) collecting a supernatant comprising hydrolyzed elastin from the suspension (a collection step 206); (V) collecting a precipitate comprising elastin from the suspension and repeating steps 202 to 206 with the precipitate to collect additional amounts of hydrolyzed elastin (a repeating step 207); and (VI) purifying the collected amounts of hydrolyzed elastin (a purifying step 208).

As shown in FIG. 7, for example, suspending step 202 may comprise suspending 5 g of elastin in 40 mL of 0.25 M aqueous oxalic acid solution. For refluxing step 203, the initial time period may be approximately one hour, and the initial temperature may be approximately 100° C. For cooling step 204 and centrifuging step 205, the cooling temperature may be approximately 4° C. Centrifuging step 205 may be performed at a rate of 3750 rpm and the subsequent time period may be approximately 5 minutes.

As shown in FIG. 7, for example, purifying step 208 may comprise transferring the collected amounts of supernatant to dialysis membranes and removing the hydrolyzed elastin from the supernatant. Dialysis may be performed against a volume of water at a dialysis temperature (e.g., such as approximately 30° C.) for a dialysis time period of between at least 5 days and approximately 7 days, adjustable relative to the volume of supernatant. As shown in FIG. 7, for example, purifying step 208 may further comprise freezing the hydrolyzed elastin to a freezing temperature (e.g., such as approximately −196° C.) and lyophilizing the frozen hydrolyzed elastin for a lyophilization time period to obtain a dry product the hydrolyzed elastin. For example, the lyophilization time period may be between at least 3 days and approximately 7 days, adjustable relative to amount of the hydrolyzed elastin.

As shown in FIG. 7, for example, method 209 may comprise: (I) maintaining a vessel at a reaction temperature (a maintaining step 210); (II) mixing the hydrolyzed elastin collected during method 201 with a basic water in the vessel to form a first solution having a target pH (a mixing step 220); (III) stirring the first solution at the reaction temperature while beginning a reaction by adding methacrylic anhydride to the vessel to form a second solution (a reaction step 230); conducting the reaction for a first time period in the absence of light while maintaining the second solution at the target pH by (A) obtaining a measured pH of the second solution at intervals during the first time period, (B) determining a difference between the measured pH and the target pH at each one of the intervals, and (C) adding a base to the vessel at each one of the intervals so that the measured pH equals the target pH (a reaction step 240); and (IV) removing unreacted methacrylic anhydride after the first time period (a removing step 250).

As shown in FIGS. 6 and/or 7, for example, maintaining step 210 may comprise maintaining a temperature bath (e.g., an electrothermal thermostatic water bath, not shown) at the reaction temperature and placing a vessel 211 in the temperature bath. The reaction temperature may be automatically maintained by a temperature control system of the temperature bath. For step 210, the reaction temperature may be room temperature, such as approximately 20° C., or between approximately 18° C. and approximately 25° C., or between 18° C. and 25° C.

As shown in FIGS. 6 and/or 7, for example, mixing step 220 may comprise forming a first solution 221 in vessel 211 by adding an amount of hydrolyzed elastin to vessel 211 and mixing it with a reactant in vessel 211. For example, mixing step 220 may comprise forming first solution 221 by dissolving 400 mg of hydrolyzed elastin in 80 mL of basic water; adding 2 M sodium hydroxide to vessel 211 at a flow rate of approximately 1 mL/min until the target pH reaches 10; and stirring the contents of vessel 211 at a stir rate (e.g., such as approximately 600 rpm) with a hotplate magnetic stirrer 222. While vessel 211 is maintained at the temperature and its contents are being stirred at the stir rate with hotplate magnetic stirrer 222, reaction step 230 may comprise adding 890 μL of methacrylic anhydride to vessel 211 at a flowrate of approximately 100 μL/min to form a second solution 231 in vessel 211. As shown in FIG. 2, for example, the methacrylic anhydride may be added dropwise with a dropper 232.

As shown in FIGS. 6 and/or 7, for example, reaction step 240 may comprise wrapping vessel 211 in a light blocking material, such as aluminum foil, and/or performing step 240 in a light-free environment, such as a dark room. As further shown, reaction step 240 may comprise obtaining the measured pH of second solution 231 with a pH sensor and comparing it to the target pH of second solution 131 at regular intervals during the first time period. As shown in FIGS. 6 and/or 7, for example, reaction step 240 also may comprise adding the base, such as 2 M sodium hydroxide, to second solution 231 at each interval so that its measured pH equals its target pH of 10 during the first time period. For example, the intervals may comprise 10-minute intervals and the first time period may be conducted for approximately 2 hours.

As shown in FIGS. 6 and/or 7, for example, removing step 250 may comprise transferring second solution 231 to dialysis membranes 251 and removing unreacted methacrylic anhydride from second solution 231 with dialysis membranes 251. Dialysis may be performed against a volume of water 252 at a dialysis temperature (e.g., such as approximately 30° C.) for a dialysis time period of between at least 5 days and approximately 7 days, adjustable relative to the volume of second solution 231. As shown in FIGS. 6 and/or 7, for example, removing step 250 may further comprise freezing second solution 231 to a freezing temperature (e.g., such as approximately −196° C.) and lyophilizing the frozen second solution 231 for a lyophilization time period to obtain a dry product pH-responsive polymer 253. For example, the lyophilization time period may be between at least 3 days and approximately 7 days, adjustable relative to the volume of solution 231.

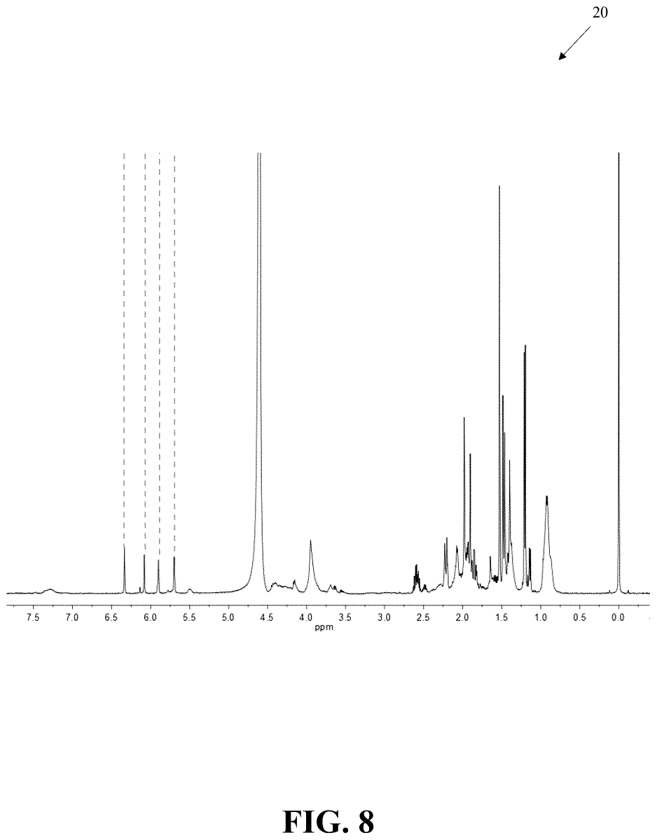

As shown in FIG. 8, for example, the formation of methacrylated hydrolyzed elastin 20 may be verified by ¹H NMR spectroscopy.

### Methacrylated Hyaluronic Acid and Methods of Synthesizing the Same

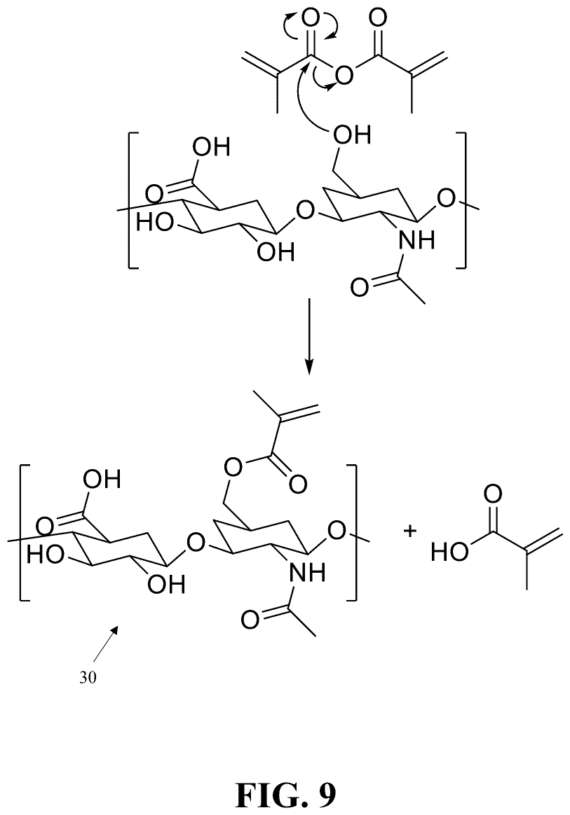

Now described with reference to FIGS. 9 to 12 is a third pH-responsive polymer or “methacrylated hyaluronic acid.” As shown in FIGS. 9 and/or 10, for example, a methacrylated hyaluronic acid 30 may comprise a synthesized polymer prepared by controlling the pH of a synthesis reaction with hyaluronic acid, resulting in a third type of pH-responsive polymer that has been selectively modified and further processed into a third dry product according to this disclosure.

Methacrylated hyaluronic acid 30 may be a pH-responsive polymer comprising hyaluronic acid with selectively modified and unmodified functional groups. As shown in FIG. 9, for example, methacrylated hyaluronic acid may comprise a compound of formula (III), wherein: (A) each R Group may attach to a primary hydroxyl group that has been reacted though an acyl substitution mechanism, allowing a methacryloyl group to attach; and (B) each hydroxyl group may not have reacted though an acyl substitution mechanism, allowing the hydroxyl group to remain as its unreacted functional group.

As shown in FIG. 9, for example, methacrylated hyaluronic acid 30 may comprise a compound of formula (III) comprising:

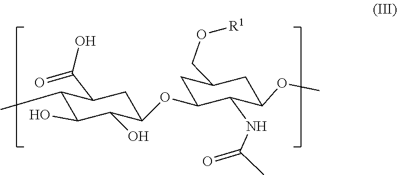

wherein:
the compound comprises a hyaluronic acid backbone;
R¹ is selected from a group consisting of:

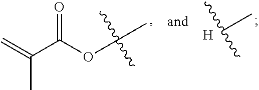

and
“ [symbol] ” indicates the point of attachment.

As shown in FIG. 9, for example, methacrylated hyaluronic acid 30 may comprise a compound of formula (III) comprising: (A) primary hydroxyl groups that have been methacrylated; or (B) carboxyl, primary hydroxyl, secondary hydroxyl, and amide groups that were not methacrylated.

The methacrylated and unmethacrylated functional groups of methacrylated hyaluronic 30 may be selected by controlling the pH level of the synthesizing reaction. As shown in FIGS. 9 and/or 10, for example, by maintaining pH levels during the synthesizing reaction: (A) the modified functional groups of methacrylated hyaluronic acid 30 may comprise methacrylated primary alcohol groups; and (B) the unmodified functional groups of methacrylated hyaluronic acid 30 may comprise (i) unreacted amide groups, (ii) unreacted carboxyl groups, or (iii) unreacted primary and secondary hydroxyl groups.

To provide additional examples for methacrylated hyaluronic acid 30, the compound of formula (III) also may comprise:

| where: | |
|---|---|
| R¹ is | 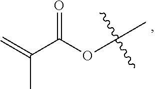 [SMILES: *OC(=O)C(=C)C] |
| or where: | |
| R¹ is | 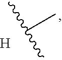 |

each of the above being examples of methacrylated hyaluronic acid 30 according to this disclosure.

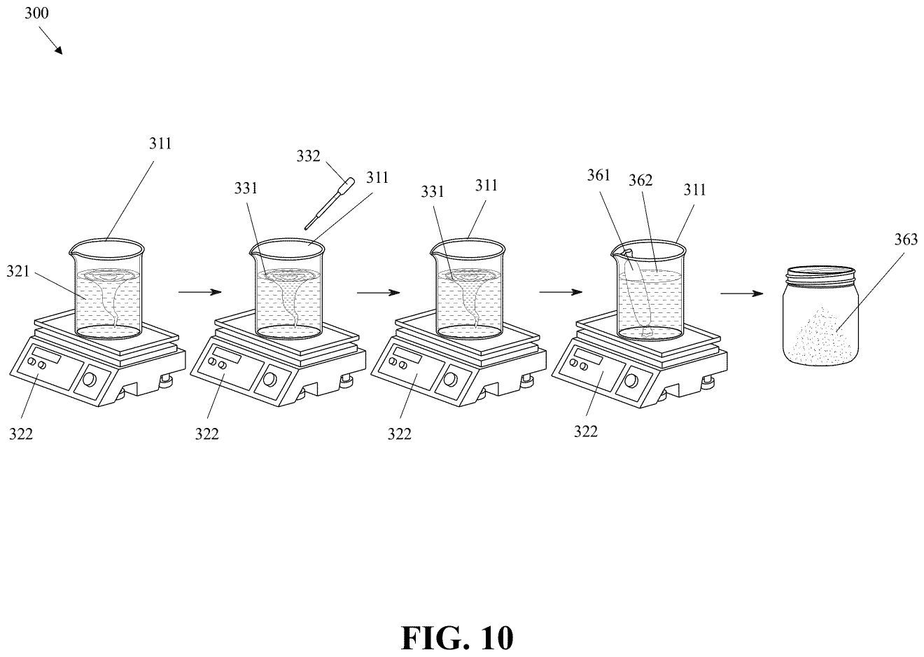

Aspects of an exemplary method 300 of synthesizing methacrylated hyaluronic acid 30 are now described with continued reference to FIGS. 9 to 12. Method 300 may comprise modifying hyaluronic acid 30 by methacrylating its functional groups. As shown in FIGS. 9, 10, and/or 11, for example, method 300 may comprise synthesizing methacrylated hyaluronic acid 30 by methacrylating hyaluronic acid with methacrylic anhydride or an equivalent thereof. As shown in FIGS. 9, 10, and/or 11, for example, method 300 may comprise reacting hyaluronic acid with methacrylic anhydride and obtaining a basic pH of the resulting solution to achieve selective modification of its functional groups.

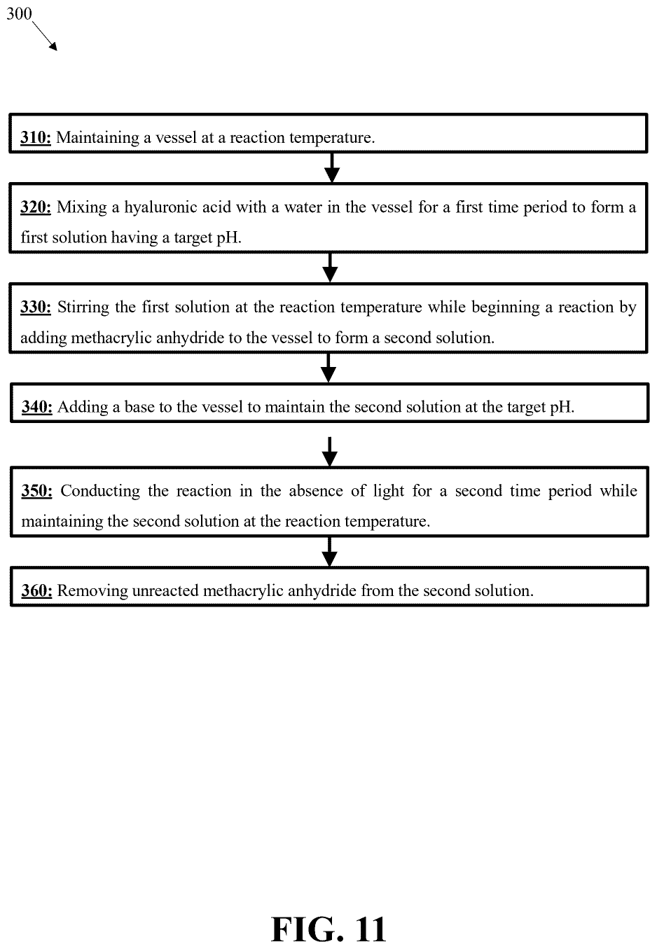

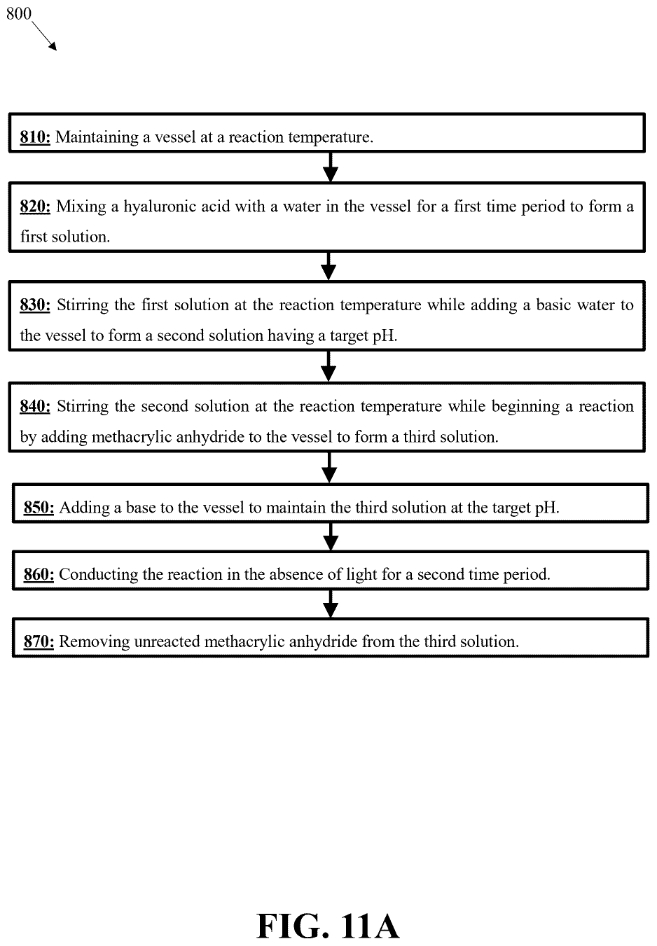

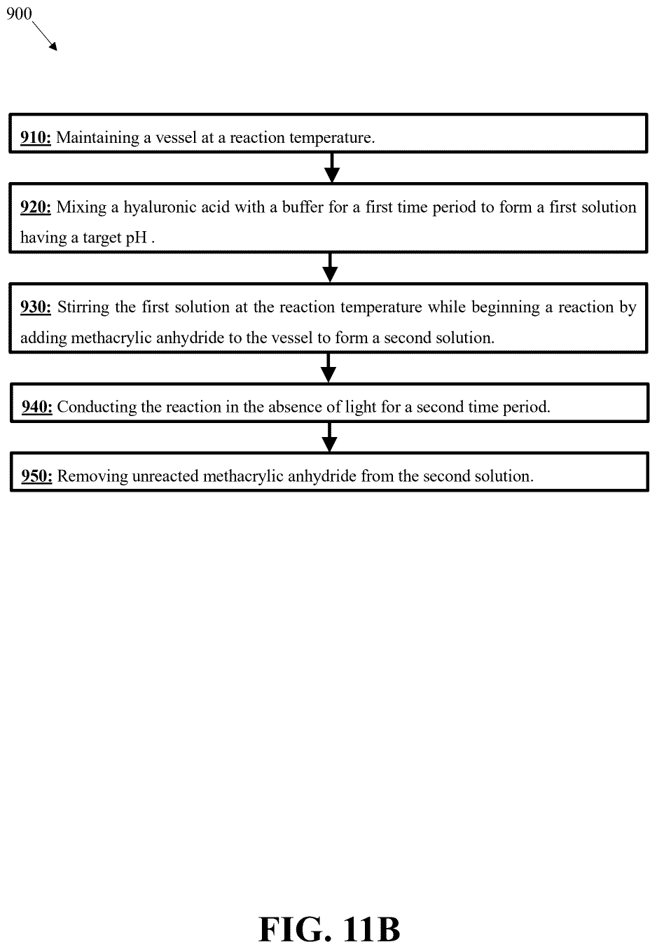

As shown in FIG. 11, for example, method 300 may comprise: (I) maintaining a vessel at a reaction temperature (a maintaining step 310); (II) mixing a hyaluronic acid with water in the vessel for a first time period to form a first solution having a target pH (a mixing step 320); (III) stirring the first solution at the reaction temperature while (A) beginning a reaction by adding methacrylic anhydride to the vessel to form a second solution (a reaction step 330); (B) adding a base to the vessel to maintain the second solution at the target pH (a reaction step 340); (C) conducting the reaction in the absence of light for a second time period while maintaining the second solution at the reaction temperature (a reaction step 350); and (IV) removing unreacted methacrylic anhydride from the second solution (a removing step 360).

As shown in FIGS. 10 and/or 11, for example, maintaining step 310 may comprise maintaining a temperature bath (e.g., an electrothermal thermostatic water bath, not shown) at the reaction temperature and placing a vessel 311 in the temperature bath. The reaction temperature may be automatically maintained by a temperature control system of the temperature bath. For step 310, the reaction temperature may be approximately 4° C., or between approximately 0° C. and approximately 10° C., or between 0° C. and 10° C.

As shown in FIGS. 10 and/or 11, for example, mixing step 320 may comprise forming a first solution 321 by adding an amount of hyaluronic acid sodium salt to vessel 311 and mixing it with an amount of reactant in vessel 311. For example, mixing step 320 may comprise forming first solution 321 by dissolving 500 mg of hyaluronic acid sodium salt in 50 mL of water in vessel 311 at 4° C. to obtain a concentration of 10 mg/mL for a first time period and stirring the contents of vessel 311 at a stir rate (e.g., such as approximately 600 rpm) with a hotplate magnetic stirrer 322. The first time period may be between at least 16 hours and approximately 24 hours.

While vessel 311 is maintained at the reaction temperature and its contents are being stirred at the stir rate with hotplate magnetic stirrer 322, reaction step 330 may comprise adding methacrylic anhydride to vessel 311 to form a second solution 331. For example, the methacrylic anhydride may be added to vessel 311 at a flowrate of approximately 100 μL/min to form second solution 311. Reaction step 340 may comprise obtaining a measured pH of second solution 331 with a pH sensor and adding the base, such as 5 M sodium hydroxide, to vessel 311 at a flow rate of approximately 1 mL/min so that the measured pH of second solution 331 is equal to the target pH of 10. As shown in FIG. 10, for example, the 5 M sodium hydroxide may be added dropwise with a dropper 332. For step 340, as with step 310, the reaction temperature may be approximately 4° C., or between approximately 0° C. and approximately 10° C., or between 0° C. and 10° C.

As shown in FIGS. 10 and/or 11, for example, reaction step 350 may comprise wrapping vessel 311 in a light blocking material, such as aluminum foil, and/or performing reaction step 350 in a light-free environment, such as a dark room. For example, the second time period may be approximately 24 hours.

As shown in FIGS. 10 and/or 11, for example, removing step 360 may comprise transferring second solution 331 to dialysis membranes 361 and removing unreacted methacrylic anhydride from second solution 331 with dialysis membranes 361. Dialysis may be performed against a volume of water 362 at a dialysis temperature (e.g., such as approximately 40° C.) for a dialysis time period between at least 5 days and approximately 7 days, adjustable relative to the volume of second solution 331. As shown in FIGS. 10 and 11, for example, removing step 360 may further comprise freezing solution 331 to a freezing temperature (e.g., such as approximately −196° C.) and lyophilizing the frozen solution 331 for a lyophilization time period to obtain a dry product pH-responsive polymer 363. For example, the lyophilization time period may occur for a time period between at least 3 days and approximately 5 days, adjustable relative to the volume of solution 331.

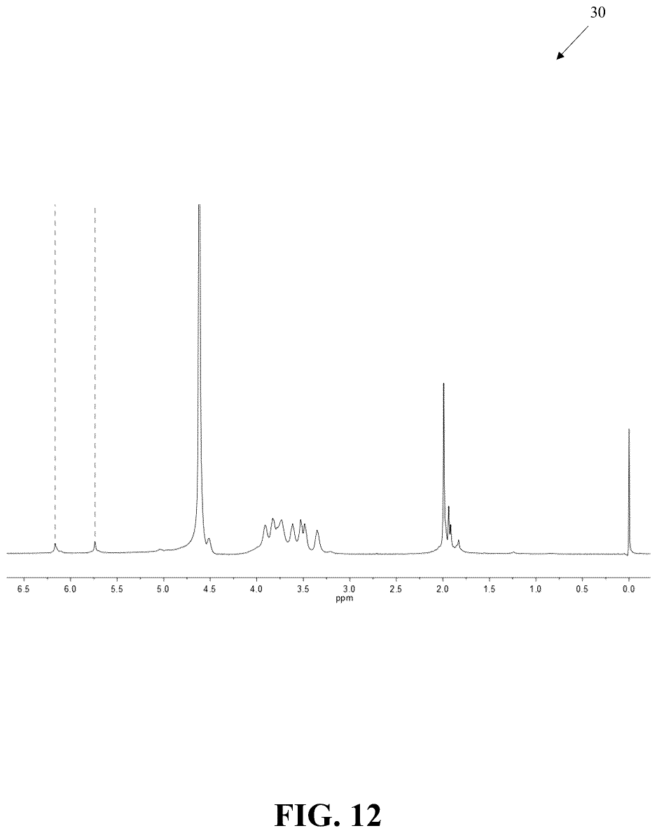

As shown in FIG. 12, for example, the formation of methacrylated hyaluronic acid 30 may be verified by ¹H NMR spectroscopy.

Alternative methods of synthesizing methacrylated hyaluronic acid 30 are contemplated. By way of example, aspects of one exemplary method 800 of synthesizing methacrylated hyaluronic acid 30 are now described with continued reference to FIGS. 9, 10, 11A, and/or 12. As shown in FIG. 11A, for example, method 800 may comprise: (I) maintaining a vessel (e.g., like vessel 311 of FIG. 10) at a reaction temperature (a maintaining step 810); (II) mixing a hyaluronic acid with a water in the vessel for a first time period to form a first solution (a mixing step 820); (III) stirring the first solution at the reaction temperature while adding a basic water solution to form a second solution having a target pH (a forming step 830); (IV) stirring the second solution at the reaction temperature while (A) beginning the reaction by adding methacrylic anhydride to the vessel to form a third solution (a reaction step 840), (B) adding a base to the vessel to maintain the third solution at the target pH (a reaction step 850), (C) conducting the reaction in the absence of light for a second time period (a reaction step 860); and (V) removing unreacted methacrylic anhydride from the third solution (a removing step 870).

As shown in FIGS. 10 and/or 11A, for example, maintaining step 810 may comprise maintaining a temperature bath (e.g. an ice/water bath, or an electrothermal cryogenic bath, not shown) or a temperature-controlled environment (e.g. a refrigerator or cold room, also not shown) and placing the vessel in the temperature bath or the temperature-controlled environment. The reaction temperature of the vessel may be manually or automatically maintained by a temperature control system of the temperature bath or the temperature-controlled environment. For step 810, the reaction temperature may be comparable to that of step 310, such as approximately 4° C., or between approximately 2° C. and approximately 10° C., or between 2° C. and 10° C.

As shown in FIGS. 10 and/or 11A, for example, mixing step 820 may comprise forming the first solution by adding an amount of hyaluronic acid sodium salt to the vessel and mixing it with a solvent in the vessel. For example, mixing step 820 may comprise dissolving 1 g of hyaluronic acid sodium salt in 100 mL water at a concentration of 10 mg/mL during the first time period while maintaining the reaction temperature described above (e.g., at approximately 4° C.) and stirring the first solution at stir rate (e.g. such as approximately 600 rpm) with a magnetic stirrer (e.g., like stirrer 322 of FIG. 10). In keeping with mixing step 320, first time period for mixing step 820 may be between at least 16 hours and approximately 24 hours.

As shown in FIGS. 10 and/or 11A, for example, forming step 830 may comprise forming the second solution by maintaining the first solution at the reaction temperature and stirring it at the stir rate while adding 5M sodium hydroxide in 10 μL aliquots until the second solution reaches a target pH of 8.5.

As shown in FIGS. 10 and/or 11A, for example, reaction step 840 may comprise forming the third solution by maintaining the second solution at the reaction temperature and stirring it at the stir rate while adding 7.5 mL of methacrylic anhydride to the vessel.

As shown in FIGS. 10 and/or 11A, for example, reaction step 850 may comprise maintaining the third solution at the target pH may comprise obtaining a measured pH of the third solution with a pH sensor and adding a base to the vessel, such as 5M sodium hydroxide, in 10 μL aliquots so that the measured pH of the third solution is equal to the target pH of 8.5.

As shown in FIGS. 10 and/or 11A, for example, reaction step 860 may comprise wrapping the vessel in a light blocking material, such as aluminum foil, and/or performing step 860 in a light-free environment, such as a dark room. In step 860, the second time period may be approximately 24 hours.

As shown in FIGS. 10 and/or 11A, for example, removing step 870 may comprise transferring the third solution to dialysis membranes (e.g., like dialysis membranes 361 of FIG. 10) and removing unreacted methacrylic anhydride from the third solution by dialysis with the dialysis membranes. Dialysis may be performed against a volume of water (e.g., like volume 362 of FIG. 10) at a dialysis temperature (e.g., such as approximately 40° C.) for a dialysis time period of between two and 3 days. As shown in FIGS. 10 and 11A, for example, removing step 870 may further comprise freezing the third solution to a freezing temperature (e.g., such as approximately −196° C.) and lyophilizing the frozen third solution for a lyophilization time period to obtain a dry product pH-responsive polymer 363. For example, the lyophilization time period may be between at least 3 days and approximately 7 days, adjustable relative to the volume of the third solution.

In keeping with above, as shown in FIG. 12, for example, the formation of methacrylated hyaluronic acid 30 may be verified by ¹H NMR spectroscopy.

By way of example, aspects of another exemplary method 900 of synthesizing methacrylated hyaluronic acid 30 are now described with continued reference to FIGS. 9, 10, 11B, and/or 12. As shown in FIG. 11A, for example, method 900 may comprise: (I) maintaining a vessel (e.g., like vessel 311 of FIG. 10) at a reaction temperature (a maintaining step 910); (II) mixing a hyaluronic acid with a buffer for a first time period to form a first solution having a target pH (a mixing step 920); (III) stirring the first solution at the reaction temperature while beginning a reaction by adding methacrylic anhydride to the vessel to form a second solution (a reaction step 930); (IV) conducting the reaction in the absence of light for a second time period (a reaction step 940); and (V) removing unreacted methacrylic anhydride from the second solution (a removing step 950).

As shown in FIGS. 10 and/or 11B, for example, maintaining step 910 may comprise maintaining a temperature bath (e.g. an ice/water bath, or an electrothermal cryogenic bath, not shown) or a temperature-controlled environment (e.g. a refrigerator or cold room, also not shown) and placing the vessel in the temperature bath or the temperature-controlled environment. The reaction temperature of the vessel may be manually or automatically maintained by a temperature control system of the temperature bath or the temperature-controlled environment. For step 810, the reaction temperature may be comparable to that of step 310, such as approximately 4° C., or between approximately 2° C. and approximately 10° C., or between 2° C. and 10° C.

As shown in FIGS. 10 and/or 11B, for example, mixing step 920 may comprise forming the first solution by adding an amount of hyaluronic acid sodium salt to the vessel and mixing it with a buffer solution in the vessel. For example, mixing step 920 may comprise forming the first solution by dissolving 1 g of hyaluronic acid sodium salt in a buffer solution comprising or consisting of 2.86 g of sodium carbonate, 7.56 g of sodium bicarbonate, and 100 mL of deionized water at a concentration of 10 mg/mL during the first time period while maintaining the reaction temperature described above (e.g., at approximately 4° C.) and stirring the first solution at a stir rate (e.g. such as approximately 600 rpm) with a magnetic stirrer (e.g., like stirrer 322 of FIG. 10). In keeping with mixing steps 320, 820, the first time period for mixing step 920 may be between at least 16 hours and approximately 24 hours.

As shown in FIGS. 10 and/or 11B, for example, reaction step 930 may comprise forming the second solution by maintaining the first solution at the reaction temperature and stirring it the stir rate while adding 7.5 mL of methacrylic anhydride to the vessel.

As shown in FIGS. 10 and/or 11B, for example, reaction step 940 may comprise wrapping the vessel in a light blocking material, such as aluminum foil, and/or performing step 940 with the second solution in a light-free environment, such as a dark room. In step 940, the second time period may be approximately 24 hours.

As shown in FIGS. 10 and/or 11B, for example, removing step 950 may comprise transferring the second solution to dialysis membranes (e.g., like dialysis membranes 361 of FIG. 10) and removing unreacted methacrylic anhydride from the second solution by dialysis with the dialysis membranes. Dialysis may be performed against a volume of water (e.g., like volume 362 of FIG. 10) at a dialysis temperature (e.g., such as approximately 40° C.) for a dialysis time period of between two and 3 days. As shown in FIGS. 10 and 11B, for example, removing step 950 may further comprise freezing the second solution to a freezing temperature (e.g., such as approximately −196° C.) and lyophilizing the frozen second solution for a lyophilization time period to obtain a dry product pH-responsive polymer 363. For example, the lyophilization time period may be between at least 3 days and approximately 7 days, adjustable relative to the volume of the second solution.

In keeping with above, as shown in FIG. 12, for example, the formation of methacrylated hyaluronic acid 30 may be verified by ¹H NMR spectroscopy.

### Polymer A and Methods of Synthesizing the Same

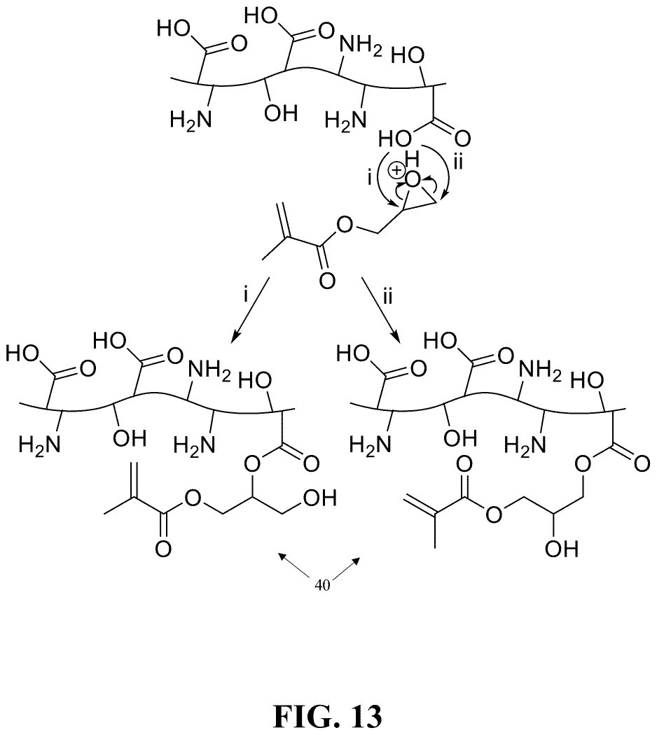

Now described with reference to FIGS. 13-16 is a fourth pH-responsive polymer or “Polymer A.” As shown in FIGS. 13 and/or 14, for example, a Polymer A 40 may comprise a synthesized polymer prepared by controlling the pH of a synthesis reaction with Gelatin Type A, resulting in a fourth different type of pH-responsive polymer that has been selectively modified and further processed into a fourth dry product according to this disclosure.

Polymer A 40 be a pH-responsive methacrylated gelatin polymer comprising Gelatin Type A with selectively modified and unmodified functional groups. As show in FIG. 13, for example, Polymer A 40 may comprise a compound of formula (IV), wherein: (A) each R Group may attach to a carboxyl group or a hydroxyl group that has been reacted though an epoxide ring-opening mechanism, allowing a methacrylate group to attach; (B) each carboxyl or hydroxyl group may not have reacted though an epoxide ring-opening mechanism, allowing the carboxyl or hydroxyl group to remain as its respective functional group; and (C) the amine group may be reacted through an epoxide ring-opening mechanism, allowing a methacrylate group to attach.

As shown in FIG. 13, for example, Polymer A may comprise a compound of formula (IV) comprising:

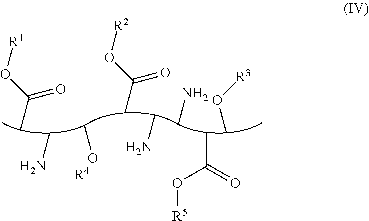

wherein:
the compound comprises a gelatin backbone;
R¹, R², R³, R⁴, and R⁵ are selected from a group consisting of:

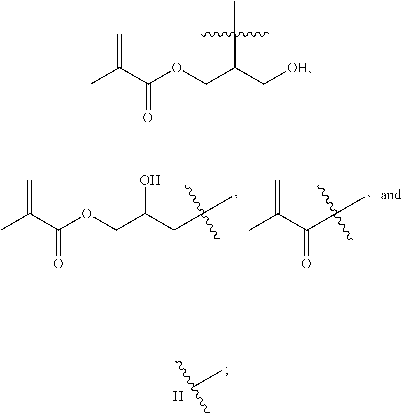

at least one of R¹, R², R³, R⁴, and R⁵ is not

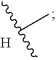

and
“ [symbol] ” indicates the point of attachment.

As shown in FIG. 13, for example, Polymer A 40 may comprise a compound of formula (IV) comprising: (A) modified carboxyl groups, modified hydroxyl groups, unmodified carboxyl groups, unmodified hydroxyl groups, and unmodified amine groups; (B) carboxyl and hydroxyl groups that have been methacrylated; or (C) carboxyl, hydroxyl, and amine groups that were not methacrylated.

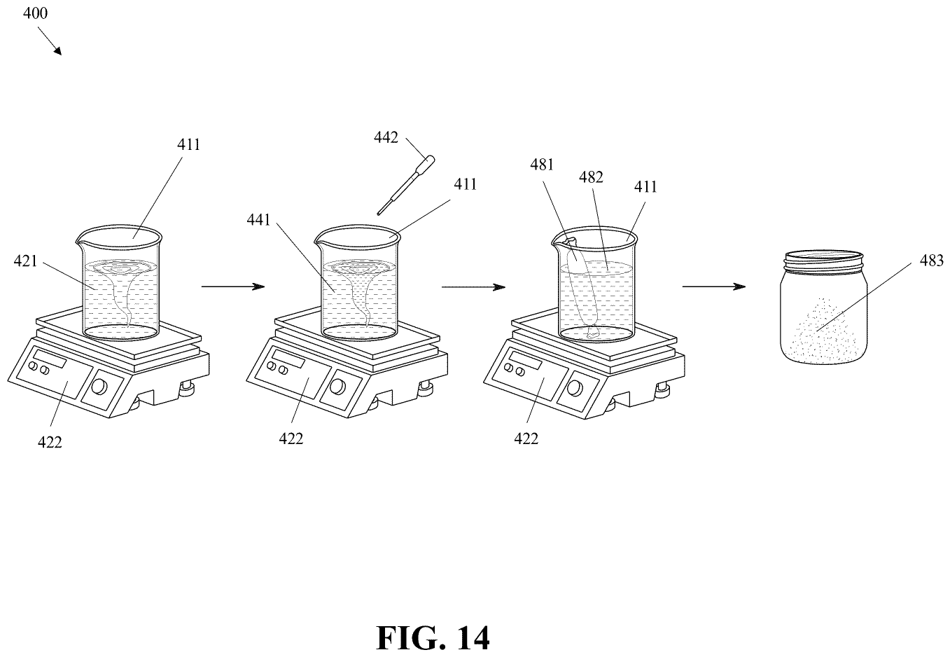

The modified and unmodified functional groups of Polymer A 40 may be selected by controlling the pH level of the synthesizing reaction. As shown in FIGS. 13, 14, and/or 15, for example, by maintaining pH levels during the synthesizing reaction: (A) the modified functional groups of Polymer A 40 may comprise (i) methacrylated carboxyl groups and methacrylated hydroxyl groups or (ii) methacrylated amine groups and methacrylated hydroxyl groups; and (B) the unmodified functional groups of Polymer A 40 may comprise (i) unreacted amine groups (ii) unreacted carboxyl groups, or (iii) unreacted hydroxyl groups.

To provide additional examples for Polymer A 40, the compound of formula (IV) also may comprise:

| where: | |
|---|---|
| R¹ is | 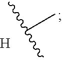 |
| R² is | 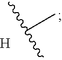 |
| R³ is | 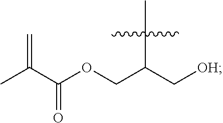 [SMILES: *C(CO)COC(=O)C(=C)C] |
| R⁴ is | 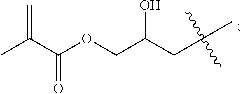 [SMILES: *CC(O)COC(=O)C(=C)C] |
| and | |
| R⁵ is | 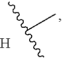 |
| or where: | |
| R¹ is | 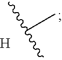 |
| R² is | 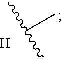 |
| R³ is | 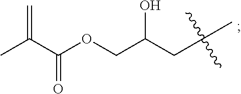 [SMILES: *CC(O)COC(=O)C(=C)C] |
| R⁴ is | 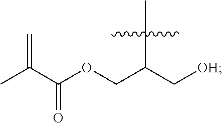 [SMILES: *C(CO)COC(=O)C(=C)C] |
| and | |
| R⁵ is | 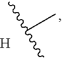 |
| or where: | |
| R¹ is | 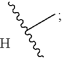 |
| R² is | 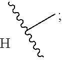 |
| R³ is | 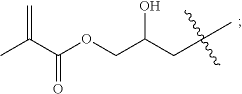 [SMILES: *CC(O)COC(=O)C(=C)C] |
| R⁴ is | 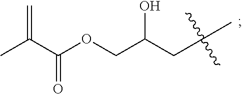 [SMILES: *CC(O)COC(=O)C(=C)C] |
| and | |
| R⁵ is | 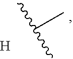 |
| or where: | |
| R¹ is | 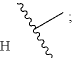 |
| R² is | 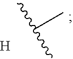 |
| R³ is | 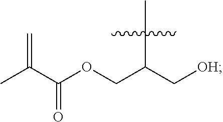 [SMILES: *C(CO)COC(=O)C(=C)C] |
| R⁴ is | 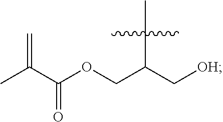 [SMILES: *C(CO)COC(=O)C(=C)C] |
| and | |
| R⁵ is | 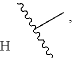 |
| or where: | |
| R¹ is | 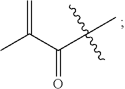 [SMILES: *C(=O)C(=C)C] |
| R² is | 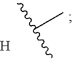 |
| R³ is | 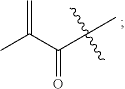 [SMILES: *C(=O)C(=C)C] |
| R⁴ is | 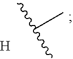 |
| and | |
| R⁵ is | 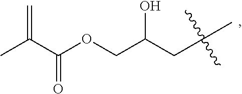 [SMILES: *CC(O)COC(=O)C(=C)C] |
| or where: | |
| R¹ is | 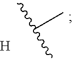 |
| R² is | 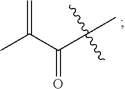 [SMILES: *C(=O)C(=C)C] |
| R³ is | 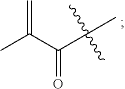 [SMILES: *C(=O)C(=C)C] |
| R⁴ is | 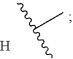 |
| and | |
| R⁵ is | 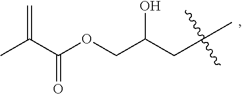 [SMILES: *CC(O)COC(=O)C(=C)C] |
| or where: | |
| R¹ is | 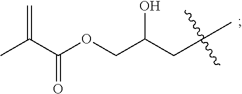 [SMILES: *CC(O)COC(=O)C(=C)C] |
| R² is | 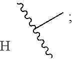 |
| R³ is | 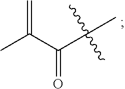 [SMILES: *C(=O)C(=C)C] |
| R⁴ is | 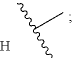 |
| and | |
| R⁵ is | 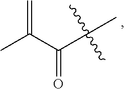 [SMILES: *C(=O)C(=C)C] |
| or where: | |
| R¹ is | 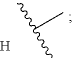 |
| R² is | 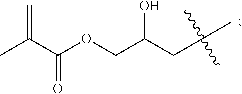 [SMILES: *CC(O)COC(=O)C(=C)C] |
| R³ is | 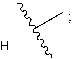 |
| R⁴ is | 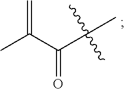 [SMILES: *C(=O)C(=C)C] |
| and | |
| R⁵ is | 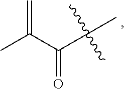 [SMILES: *C(=O)C(=C)C] |
| or where: | |
| R¹ is | 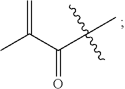 [SMILES: *C(=O)C(=C)C] |
| R² is | 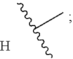 |
| R³ is | 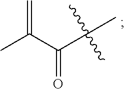 [SMILES: *C(=O)C(=C)C] |
| R⁴ is | 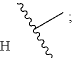 |
| and | |
| R⁵ is | 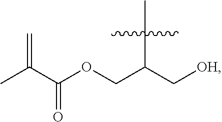 [SMILES: *C(CO)COC(=O)C(=C)C] |
| or where: | |
| R¹ is | 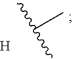 |
| R² is | 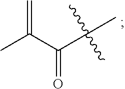 [SMILES: *C(=O)C(=C)C] |
| R³ is | 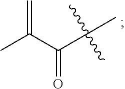 [SMILES: *C(=O)C(=C)C] |
| R⁴ is | 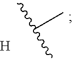 |
| and | |
| R⁵ is | 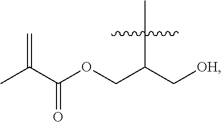 [SMILES: *C(CO)COC(=O)C(=C)C] |
| or where: | |
| R¹ is | 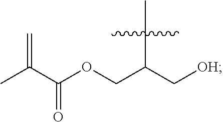 [SMILES: *C(CO)COC(=O)C(=C)C] |
| R² is | 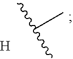 |
| R³ is | 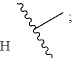 |
| R⁴ is | 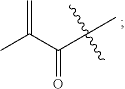 [SMILES: *C(=O)C(=C)C] |
| and | |
| R⁵ is | 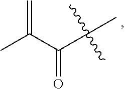 [SMILES: *C(=O)C(=C)C] |
| or where: | |
| R¹ is | 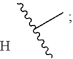 |
| R² is | 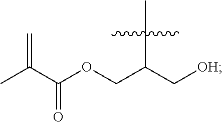 [SMILES: *C(CO)COC(=O)C(=C)C] |
| R³ is | 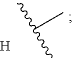 |
| R⁴ is | 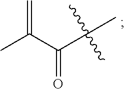 [SMILES: *C(=O)C(=C)C] |
| and | |
| R⁵ is | 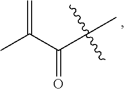 [SMILES: *C(=O)C(=C)C] |

each of the above being examples of Polymer A 40 according to this disclosure.

Aspects of an exemplary method 400 of synthesizing a Polymer A 40 are now described with continued reference to FIGS. 13 to 16. Method 400 may comprise modifying Gelatin Type A by methacrylating its functional groups. As shown in FIGS. 13, 14, and/or 16, for example, method 400 may comprise synthesizing Polymer A 40 by methacrylating the Gelatin Type A with glycidyl methacrylate or an equivalent thereof. As shown in FIGS. 13, 14, and/or 16, method 400 may comprise reacting the Gelatin Type A with glycidyl methacrylate and maintaining an acidic pH of the resulting solution to achieve selective modification of its functional groups.

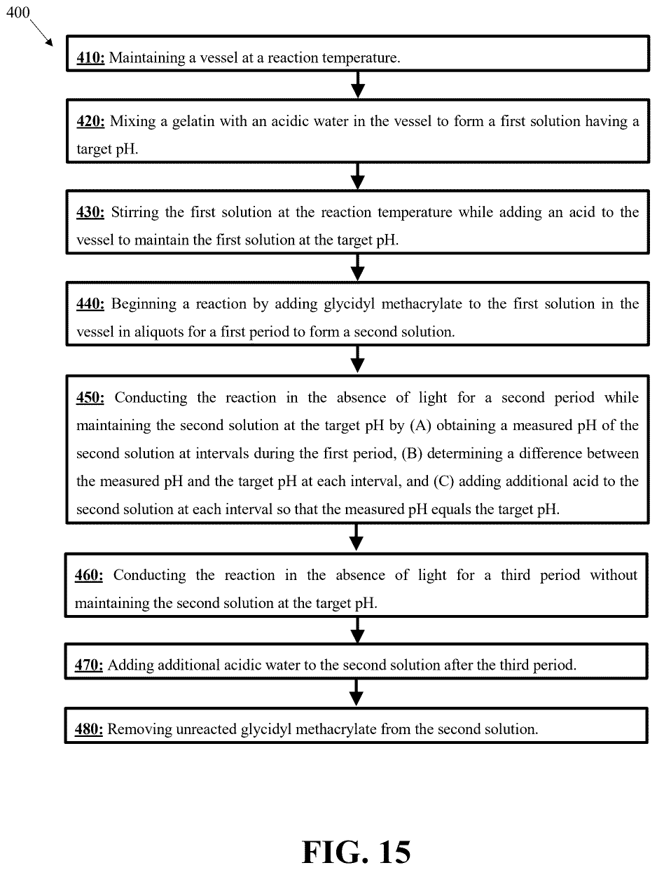

As shown in FIG. 15, for example, method 400 may comprise: (I) maintaining a vessel at a reaction temperature (a maintaining step 410); (II) mixing a gelatin (e.g., a Gelatin Type A) with an acidic water in a vessel to form a first solution having a target pH (a mixing step 420); (III) stirring the first solution at the reaction temperature while adding an acid to the vessel to maintain the first solution at the target pH (a maintaining step 430); (IV) beginning a reaction by adding glycidyl methacrylate to the first solution in the vessel in aliquots for a first time period to form a second solution (a reaction step 440); (V) conducting the reaction in the absence of light for a second time period while maintaining the second solution at the target pH by (A) obtaining a measured pH of the second solution at intervals during the second time period, (B) determining a difference between the measured pH and the target pH at each one of the intervals, and (C) adding additional acid to the second solution at each one of the intervals so that the measured pH equals the target pH (a reaction step 450); (VI) conducting the reaction for a third time period without maintaining the second solution at the target pH (a conducting step 460); (VII) adding additional acidic water to the second solution after the third time period (an adding step 470); and (VIII) removing unreacted glycidyl methacrylate from the second solution (a removing step 480).

As shown in FIGS. 14 and/or 15, for example, maintaining step 410 may comprise maintaining a temperature bath (e.g., an electrothermal thermostatic water bath, not shown) at the reaction temperature and placing a vessel 411 in the temperature bath. The reaction temperature may be automatically maintained by a temperature control system of the temperature bath. For step 410, the reaction temperature may be approximately 40° C., or between approximately 40° C. and approximately 50° C., or between 40° C. and 50° C.

As shown in FIGS. 14 and/or 15, for example, mixing step 420 may comprise forming a first solution 421 by adding an amount of Gelatin Type A to vessel 411 and mixing it with an amount of reactant in vessel 411. For example, mixing step 420 may comprise forming first solution 421 by dissolving 10 g of Gelatin Type A in 100 mL of aqueous 1 M hydrochloric acid with a pH of 3.5 in vessel 411 at a concentration of 100 mg/mL and stirring the contents of vessel 411 at a stir rate (e.g., such as approximately 800 rpm) with a hotplate magnetic stirrer 422. The pH of first solution 421 may change as the Gelatin Type A dissolves in the reactant, requiring the pH of solution 421 to be adjusted during maintaining step 430. While vessel 411 is maintained at the reaction temperature and its contents are being stirred at the stir rate with hotplate magnetic stirrer 422, maintaining step 430 may comprise measuring a pH of first solution 421 and adding the acid, such as 1 M hydrochloric acid, to vessel 411 as needed for the pH of first solution 421 to equal the target pH of 3.5, thereby correcting for any pH changes caused by dissolving the Gelatin Type A into the reactant during mixing step 420. Reaction step 440 may comprise adding glycidyl methacrylate to vessel 411 at a flowrate in equal volume aliquots at intervals during the first time period to form a second solution 441. As shown in FIGS. 14 and/or 15, for example, the flowrate may be approximately 700 μL/min, each aliquot may be approximately 20 mL added dropwise and sequentially to second solution 441 with a dropper 442, the glycidyl methacrylate may be added in thirty-minute intervals, and the first time period may be approximately 2.5 hours.

As shown in FIGS. 14 and/or 15, for example, reaction step 450 may comprise wrapping vessel 411 in a light blocking material, such as aluminum foil, and/or performing reaction step 450 in a light-free environment, such as a dark room. As further shown, reaction step 450 may comprise obtaining the measured pH of second solution 441 with a pH sensor and comparing it to the target pH of second solution 441 at regular intervals during the second time period. As shown in FIGS. 14 and/or 15, for example, reaction step 450 may comprise adding the additional acid, such as additional 1 M hydrochloric acid, to second solution 441 (e.g., dropwise) at each interval so that its measured pH equals the target pH of 3.5. For example, the intervals may comprise approximately 30-minute intervals and the second time period may be conducted for approximately 12 hours.

As shown in FIGS. 14 and/or 15, for example, conducting step 460 may comprise stirring second solution 441 at the reaction temperature in the absence of light for the third reaction time period, which may be approximately 12 hours. Adding step 470 may comprise adding an additional 10 mL of pH 3.5 acidic water to second solution 441 per 1 g Gelatin Type A after the third time period.

As shown in FIGS. 14 and/or 15, for example, removing step 480 may comprise transferring second solution 441 to dialysis membranes 481 and removing unreacted glycidyl methacrylate from second solution 441 with dialysis membranes 481. Dialysis may be performed against a volume of water 492 at a dialysis temperature (e.g., such as approximately 40° C.) for a dialysis time period of at least 5 days, adjustable relative to the volume of solution 441. As shown in FIGS. 14 and/or 15, for example, removing step 480 may further comprise freezing solution 441 to a freezing temperature (e.g., such as approximately −196° C.) and lyophilizing the frozen solution 441 for a lophilization time period to obtain a dry product pH-responsive polymer 483. For example, the lyophilization time period may be between at least 3 days and approximately 5 days, adjustable relative to the volume of solution 441.

As shown in FIG. 12, the formation of Polymer A 40 may be verified by ¹H NMR spectroscopy.

Additional aspects of Polymer A 40 may be described in U.S. Provisional Patent Application No. 63/310,416, filed Feb. 15, 2022; and International PCT Patent Application No. PCT/CA2023/050192, filed Feb. 14, 2023, with a priority claim thereto, the entireties of which are hereby incorporated by reference into this disclosure (the “Incorporated Applications”).

### Bioinks and Methods of Synthesizing the Same

According to this disclosure, one or more of first pH-responsive polymer 10, second pH-responsive polymer 20, third pH-responsive polymer 30, fourth pH-responsive polymer 40, and/or any equivalents or obvious variants thereof may be mixed with additional elements to form bioinks, such as (i) tissue-specific bioinks configured for 3D bioprinting extracellular matrices that mimic different types of biological tissue, including human tissues; and/or (ii) universal bioinks configured for 3D bioprinting similar tissue models with different types of 3D bioprinters, making the universally applicable with different 3D bioprinting techniques. Reducing the one or more pH-responsive polymers 10, 20, 30, and/or 40 into dry products 153, 253, 363, and/or 483 according to synthetization methods 100, 200, 300, and/or 400 may simplify the mixing process, allowing any bioink described herein to be efficiently optimized for particular purposes and/or more repeatably produced from longer shelf-life materials.

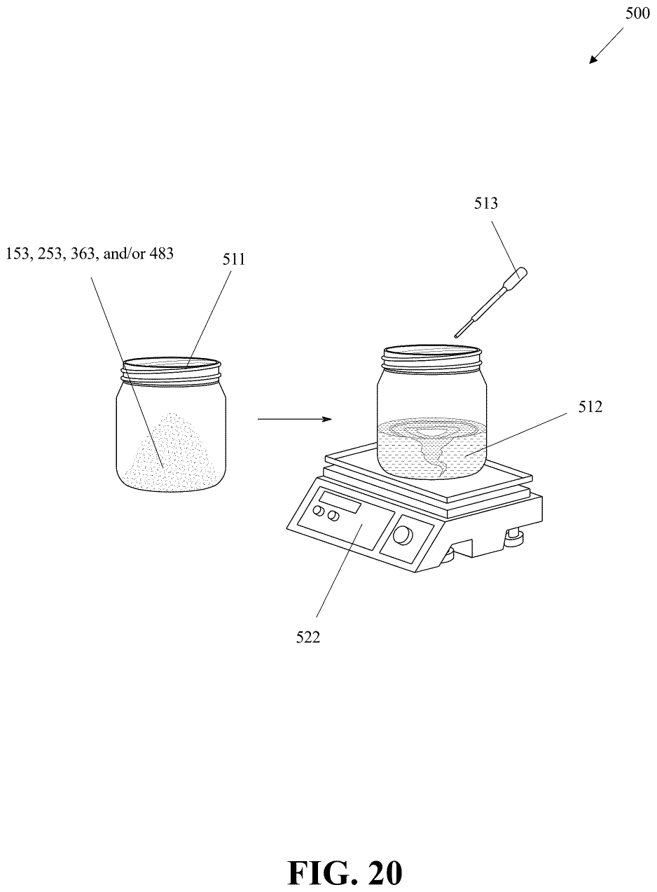

As shown in FIG. 20, for example, any bioink, tissue-specific bioink, and/or universal bioink described herein may comprise one or more pH-responsive polymers, a photoinitiator, and a solvent. The one or more pH-responsive polymers may comprise any combination of any variation of methacrylated collagen 10 described above with reference to FIGS. 1-4, any variation of methacrylated hydrolyzed elastin 20 described above with reference to FIGS. 5-8, any variation of methacrylated hyaluronic acid 30 as described above with reference to FIGS. 9-12, any variation of Polymer A 40 described above with reference to FIGS. 13-16, and/or any equivalents or obvious variants thereof.

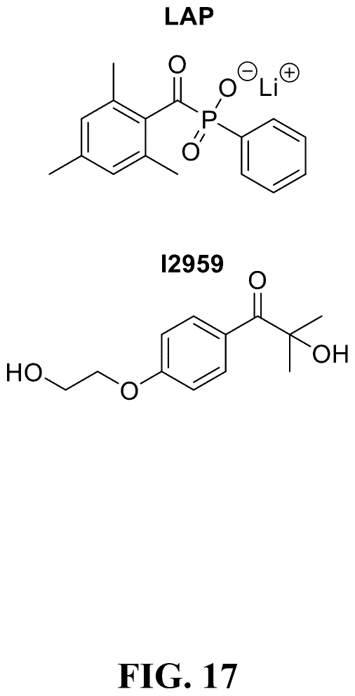

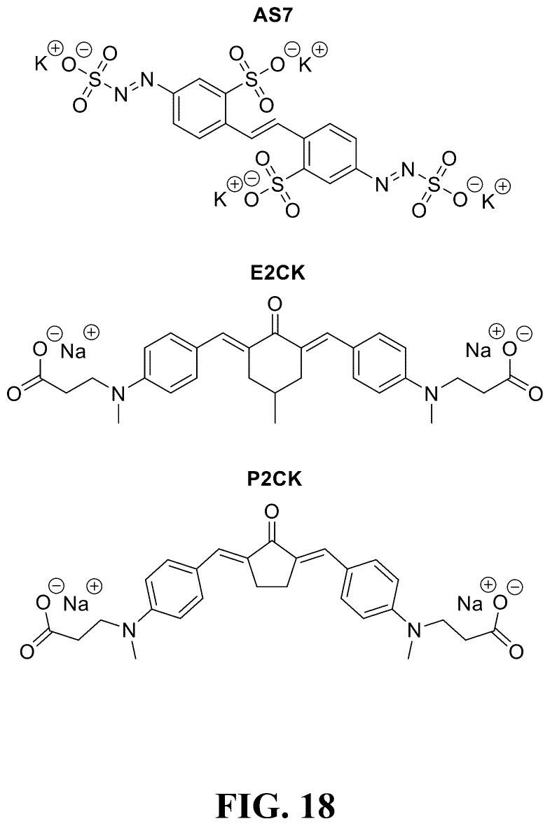

Different types of photoinitiators may be used to support different printing methods so that any bioink according to this disclosure may be described as a universal bioink because of its ability to produce similar tissue models with different types of 3D bioprinters. As shown in FIG. 17, for example, the photoinitiator may comprise a one-photon photoinitiator like those typically used with extrusion-based or DLP printing methods, such as lithium phenyl-2,4,6-trimethylbenzoylphosphinate (LAP) or 2-hydroxy-4′-(2-hydroxyethoxy)-2-methylpropiophenone (Irgacure I2959). As shown in FIG. 18, for example, the photoinitiator may comprise a two-photon photoinitiator like those typically used with two-photon polymerization printing methods, such as tetrapotassium-4,4′-(1,2-ethenediyl) bis[2-(3-sulfophenyl)diazenesulfonate](AS7), E2CK, or P2CK. Any equivalents or obvious variants of these exemplary photoinitiators also may be used.

The solvent may comprise a phosphate buffered saline, a cell culture media, and/or any equivalents or obvious variants thereof.

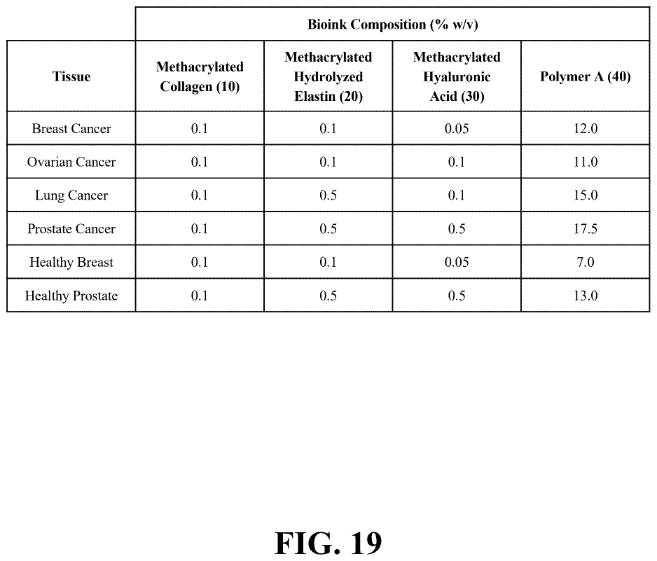

According to this disclosure, different types of tissue-specific bioinks may be composed by mixing a particular combination of one or more pH-responsive polymers (e.g., like polymers 10, 20, 30, and/or 40) with a particular photoinitiator and a particular solvent, making it possible to customize each tissue-specific bioink for the purpose of mimicking a particular type of soft tissue. As shown in FIG. 19, for example:
(i) a first tissue-specific bioink for mimicking breast cancer tissues may comprise a plurality of pH-responsive polymers comprising 0.1% w/v methacrylated collagen 10, 0.1% w/v methacrylated hydrolyzed elastin 20, 0.05% w/v methacrylated hyaluronic acid 30, and 12.0% w/v Polymer A 40;
(ii) a second tissue-specific bioink for mimicking ovarian cancer tissues may comprise a plurality of pH-responsive polymers comprising 0.1% w/v methacrylated collagen 10, 0.1% w/v methacrylated hydrolyzed elastin 20, 0.1% w/v methacrylated hyaluronic acid 30, and 11.0% w/v Polymer A 40;
(iii) a third tissue-specific bioink for mimicking lung cancer tissues may comprise a plurality of pH-responsive polymers comprising 0.1% w/v methacrylated collagen 10, 0.5% w/v methacrylated hydrolyzed elastin 20, 0.1% w/v methacrylated hyaluronic acid 30, and 15.0% w/v Polymer A 40;
(iv) a fourth tissue-specific bioink for mimicking prostate cancer tissues may comprise a plurality of pH-responsive polymers comprising 0.1% w/v methacrylated collagen 10, 0.5% w/v methacrylated hydrolyzed elastin 20, 0.5% w/v methacrylated hyaluronic acid 30, and 17.5% w/v Polymer A 40;
(v) a fifth tissue-specific bioink for mimicking healthy breast tissues may comprise a plurality of pH-responsive polymers comprising 0.1% w/v methacrylated collagen 10, 0.1% w/v methacrylated hydrolyzed elastin 20, 0.05% w/v methacrylated hyaluronic acid 30, and 7.0% w/v Polymer A 40;
(vi) a sixth tissue-specific bioink for mimicking healthy prostate tissues may comprise a plurality of pH-responsive polymers comprising 0.1% w/v methacrylated collagen 10, 0.5% w/v methacrylated hydrolyzed elastin 20, 0.5% w/v methacrylated hyaluronic acid 30, and 13.0% w/v Polymer A 40; and
(vii) any number of additional tissue-specific bioinks for mimicking other tissues may comprise alternative percentages w/v of methacrylated collagen 10, methacrylated hydrolyzed elastin 20, methacrylated hyaluronic acid 30, and/or Polymer A 40.

Examples (i) to (vii) above are not limiting unless claimed as several tissue-specific bioinks may be produced with the elements described herein for use with any type of 3D bioprinter and/or any number of different tissue types without departing from the teachings of this disclosure.

In keeping with above, any bioink, tissue-specific bioink, and/or universal bioink described herein also may be formed by mixing one or more of pH-responsive polymers (e.g., polymers 10, 20, 30, and/or 40) with GelMA type A or type B, as commonly understood; or with Polymer A 40 described above; or with Polymer B described in the Incorporated Applications as a pH responsive GelMA polymer comprising a gelatin type B with selectively modified amine groups, modified hydroxyl groups, unmodified carboxyl groups, unmodified, hydroxyl groups, and unmodified amine groups, such as a compound with amine and hydroxyl groups that have been methacrylated; and carboxyl, hydroxyl, and amine groups that were not methacrylated. For example:
(i) a first tissue-specific bioink for mimicking breast cancer tissues may comprise a plurality of pH-responsive polymers comprising 7% w/v GelMA, 0.1% w/v methacrylated collagen 10, and 0.2% methacrylated hyaluronic acid 30;
(ii) a second tissue-specific bioink for mimicking breast cancer tissues may comprise a plurality of pH-responsive polymers comprising 9% w/v Polymer B, 0.2% w/v methacrylated collagen 10, and 0.1% methacrylated hyaluronic acid 30;
(iii) a third tissue-specific bioink for mimicking breast cancer tissues may comprise a plurality of pH-responsive polymers comprising 9% w/v GelMA, and 0.1% w/v methacrylated collagen 10, and 0.05% methacrylated hyaluronic acid 30;
(iv) a fourth tissue-specific bioink for mimicking breast cancer tissues may comprise a plurality of pH-responsive polymers comprising 10% w/v GelMA, and 0.1% w/v methacrylated collagen 10; and
(v) any number of additional tissue-specific bioinks for mimicking other tissues may comprise alternative percentages w/v of GelMA, Polymer A, Polymer B, methacrylated collagen 10, methacrylated hydrolyzed elastin 20, methacrylated hyaluronic acid 30, and/or Polymer A 40.

Here again, examples (i) to (v) above are not limiting unless claimed as several tissue-specific bioinks may be produced with the elements described herein for use with any type of 3D bioprinter and/or any number of different tissue types without departing from the teachings of this disclosure.

Aspects of an exemplary method 500 for preparing a bioink are now described with pH responsive polymers 10, 20, 30, 40, and the like. As shown in FIGS. 20 and/or 21, for example, method 500 may comprise: (I) adding one or more pH-responsive polymers, a photoinitiator, and a solvent in a vessel to form a suspension in the vessel (an adding step 510); (II) mixing the suspension to form a mixture in the vessel (a mixing step 520); and (III) heating the mixture to a temperature to fully dissolve its components in the vessel (a heating step 530).

As shown in FIGS. 20 and/or 21, for example, adding step 510 may comprise: (i) dissolving a photoinitiator in a first amount of the solvent (e.g., a first amount of a phosphate buffered saline (“PBS”), a cell culture media, or water); (ii) at least partially dissolving a mixture of one or more pH-responsive polymers in a second amount of the solvent (e.g., the PBS, the cell culture media, or the water); and (iii) adding the first and second amounts to a vessel 511 to form a suspension 512 in vessel 511. In keeping with FIGS. 20 and/or 21, for example, adding step 510 may alternatively comprise (i) adding a dissolved solution of the photoinitiator to an amount of the solvent (e.g., the PBS, the cell culture media, or the water) in vessel 511 to form a mixture in vessel 511; and (ii) adding the mixture to a dry mixture of the one or more pH-responsive polymers in vessel 511 to form a suspension 512 in vessel 511. As shown in FIG. 20, for example, the dissolved solution of the photoinitiator may be added dropwise with a dropper 513.

As shown in FIGS. 20 and/or 21, for example, mixing step 520 may comprise stirring or vortex mixing suspension 512 in vessel 511. As a further example, step 520 may comprise stirring suspension 512 at a first stir rate of approximately 200 rpm until the dry products 153, 253, 363, and/or 483 are at least partially dissolved, and then stirring suspension 512 at a second stir rate of between approximately 800 rpm and approximately 1500 rpm until the mixture is formed. The temperature of heating step 530 may be approximately 50° C. As shown in FIG. 20, for example, mixing step 520 and heating step 530 may be performed with a hotplate magnetic stirrer 522.

### Methods of Measuring the Mechanical Stiffness of Bioinks

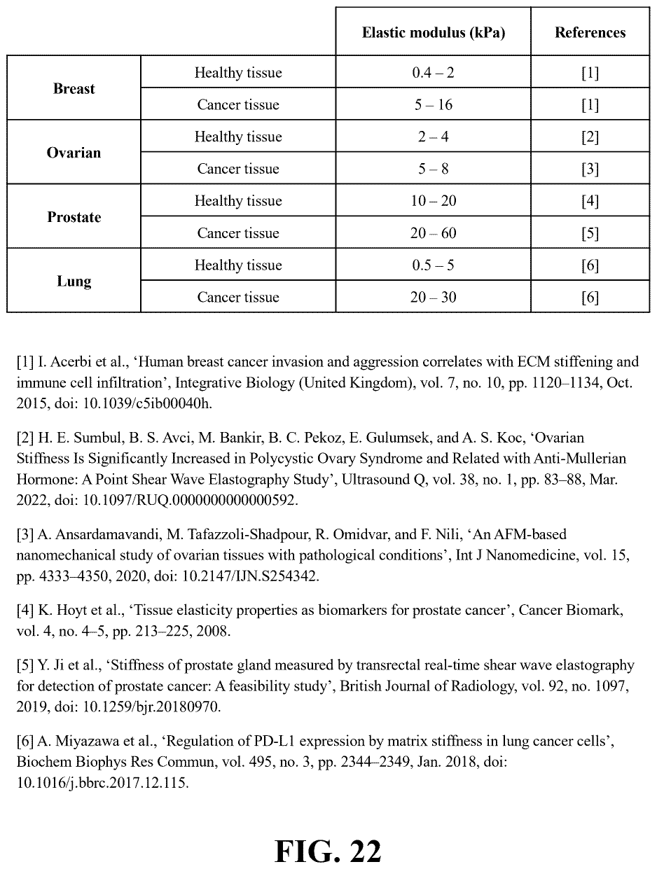

Because they include more components of the extracellular matrix of soft tissues, the bioinks described herein may be more chemically like real soft tissues, allowing for printing of extracellular matrices that more closely mimic key structural and biochemical characteristics of different types of biological tissues, such as their elastic modulus, examples of which are shown in FIG. 22 with supporting citations. Additional methods of verification may be required to ensure these characteristics may be consistently realized.

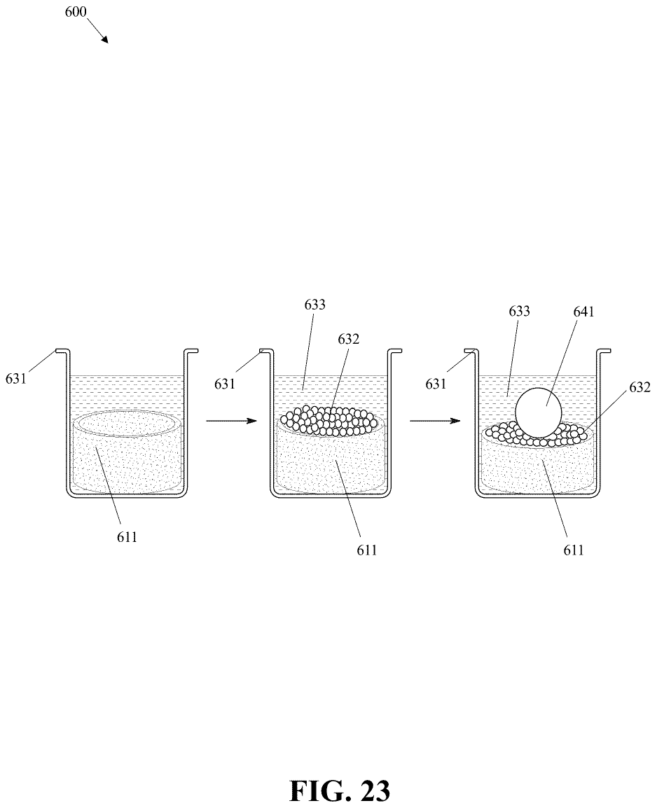

Aspects of an exemplary method 600 for measuring characteristics of bioinks are now described with reference to FIGS. 23 to 27. As shown in FIGS. 23 and/or 24, for example, method 600 may comprise: (I) preparing a hydrogel pellet of a photo-crosslinked bioink (a preparing step 610); (II) soaking the hydrogel pellet in a fluorescein sodium salt solution (a soaking step 620); (III) drop-casting fluorescent polystyrene particles onto a surface of the hydrogel pellet (a drop-casting step 630); (IV) determining measured characteristics of the hydrogel pellet, such as its elastic parameters (a measuring step 640); and (V) determining calculated characteristics of the hydrogel pellet based on its primary characteristic, such as its elastic modulus (a calculating step 650).

As shown in FIGS. 23 and/or 24, for example, preparing step 610 may comprise drop-casting an amount of bioink into a polytetrafluoroethylene (or PTFE) surface, covering the drop-casted amount with a coverslip and a spacer, and crosslinking the drop-casted amount using UV light for a first time period to form a hydrogel pellet 611. In keeping with FIG. 23, for example, hydrogel pellet 611 may be approximately 1 cm in diameter and approximately 300 to 700 μm thick, the UV light may comprise a 365 nm wavelength, and the first time period may be approximately 3 minutes.

As shown in FIGS. 23 and/or 24, for example, soaking step 620 may comprise transferring hydrogel pellet 611 and the cover slip to a well-plate, adding an amount of fluorescein sodium salt to the well-plate, adding an amount of a medium to the well-plate, and maintaining the well-plate at a temperature for a second time period. In keeping with FIG. 23, for example, the fluorescein sodium salt may be 35 μM fluorescein sodium salt in the medium, which may be phosphate buffered saline. For step 620, the temperature may be room temperature, such as approximately 20° C., or between approximately 18° C. and approximately 25° C., or between 18° C. and 25° C. The second time period may be approximately 16 hours.

As shown in FIGS. 23 and/or 24, for example, drop-casting step 630 may comprise transferring hydrogel pellet 611 and the coverslip to a microscope sample holder 631, drop-casting an amount of fluorescent polystyrene particles 632 (e.g., labeled a as suspension of red latex particles) onto hydrogel pellet 611; and (c) allowing particles 632 to settle for a third time period. In keeping with FIG. 23, for example, fluorescent polystyrene particles 632 may be suspended in an amount of water 633 and the third time period may be at least 5 minutes.

The measured characteristics of the bioink may comprise elastic parameters of hydrogel pellet 611. As shown in FIGS. 23 and/or 24, for example, measuring step 640 may comprise placing an indenter 641 of appropriate size and density onto hydrogel pellet 611, measuring a depth of an indent made on hydrogel pellet 611 with indenter 641, and measuring a thickness of hydrogel pellet 611. As shown in FIG. 23, for example, the appropriate size and density of indenter 641 may be where (i) a first ratio of the radius of indenter 641 to the thickness of hydrogel pellet 611 is between 0.3 and 12; and (ii) a second ratio of the depth of the indentation to the thickness of hydrogel pellet 611 is less than 0.6.

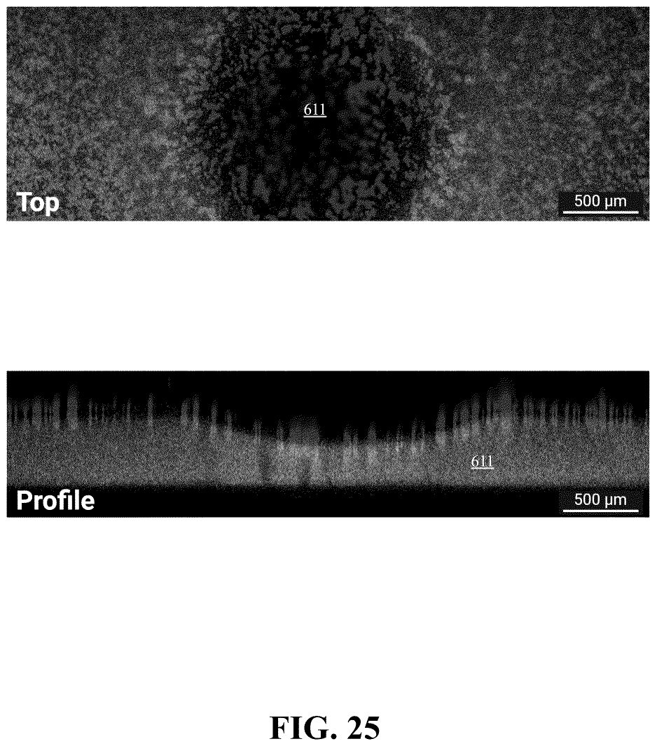

As shown in FIG. 25, for example, the radius of the indent, the depth of the indent, and the thickness of hydrogel pellet 611 may be measured in step 640 using laser scanning confocal microscopy images taken from top-down and profile views of pellet 611.

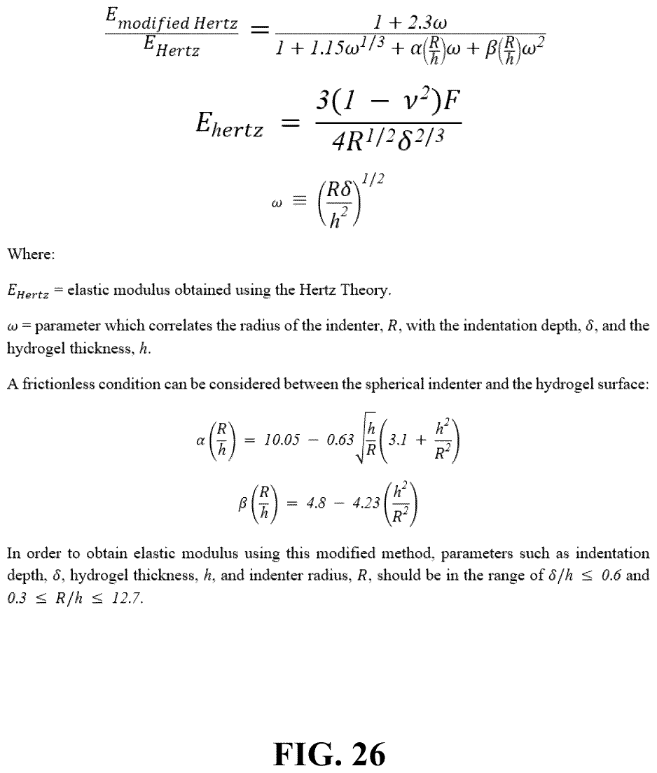

The calculated characteristics of the bioink may comprise an elastic modulus of hydrogel pellet 611. As shown in FIG. 26, for example, calculating step 650 may comprise using a modified Hertz model to calculate the elastic modulus of hydrogel pellet 611. In this example, calculating step 650 may be performed with the equation shown in FIG. 26 using variables such as the measured elastic parameters of hydrogel pellet 611 from step 640, the force applied by indenter 641 during step 640, acceleration due to gravity, a density of indenter 641, and a density of the medium (e.g., the density of phosphate buffered saline).

Exemplary benefits of tissue-specific bioinks prepared according to this disclosure are now described, including benefits associated with tissue-specific and/or universal bioinks comprising one or more pH-response polymers like methacrylated collagen 10, methacrylated hydrolyzed elastin 20, methacrylated hyaluronic acid 30, and/or Polymer A 40. When synthesized according to method 500 using one or more pH responsive polymers prepared with methods 100, 200, 300, and/or 400, the tissue-specific and/or universal bioinks of this disclosure, including those listed in FIG. 19, may be described as multicomponent compositions that improve upon commercially available bioinks by making it possible to customize each bioink for the purpose of mimicking a particular type of soft tissue using a particular 3D bioprinting technology. Because of their multicomponent compositions, the bioinks described herein (e.g., with reference to FIG. 19) may be uniquely configured to mimic a variety of different soft tissues, including the healthy and cancerous tissues listed in FIG. 22. For example, because they comprise more components of the extracellular matrix of soft tissues, the bioinks described herein may provide a more chemically similar bioink to that of real soft tissue, allow for 3D bioprinting of more representative tissue models of the extracellular matrix.

It is further contemplated that tissue models and other tissue engineering constructs printed from the described bioinks comprising one or more pH-response polymers like methacrylated collagen 10, methacrylated hydrolyzed elastin 20, methacrylated hyaluronic acid 30, and/or Polymer A 40 may simulate individual soft tissues more effectively by matching the mechanical stiffness of a particular bioink with that of a particular tissue to be 3D bioprinted therewith. As described above with reference to method 600 and FIG. 26, characterizing a mechanical stiffness (elastic modulus) bioinks like those described herein may provide a measure of comparison to human tissue that is otherwise not possible for commercially available bioinks. In addition to these benefits, whereas most commercially available bioinks are typically compatible with one type of 3D bioprinter, the bioinks described herein may be described as “universal” because the one or more pH sensitive polymers may be combined with either one-photon photoinitiators (e.g., like those of FIG. 17) or two-photon photoinitiators (e.g., like those of FIG. 18) without sacrificing their productive capabilities, making each bioink described herein likely compatible with any commercially available 3D bioprinters using extrusion-based, DLP, or two-photon technologies.

While principles of the present disclosure are described herein with reference to illustrative aspects, the disclosure is not limited thereto. Those having ordinary skill in the art and access to the teachings provided herein will recognize additional modifications, applications, aspects, and substitution of equivalents that all fall in the scope of the aspects described herein. Accordingly, the present disclosure is not to be considered as limited by the foregoing description.

Embodiments in which an exclusive property or privilege is claimed are defined as follows:

1. A compound of formula (I) 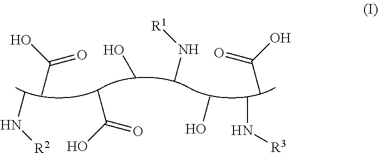
wherein:
the compound comprises a collagen backbone;
R¹, R², and R³ are selected from a group consisting of: 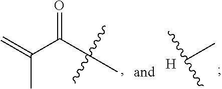
at least one of R¹, R², and R³ is not 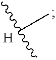
and
“ [symbol] ” indicates the point of attachment.

2. The compound of formula (I) of claim 1, wherein
R¹ is 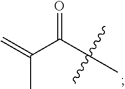 [SMILES: *C(=O)C(=C)C]
R² is 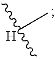
and
R³ is

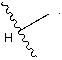

3. The compound of formula (I) of claim 1, wherein
R¹ is 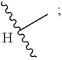
R² is 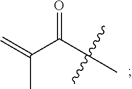 [SMILES: *C(=O)C(=C)C]
and
R³ is

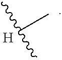

4. The compound of formula (I) of claim 1, wherein
R¹ is 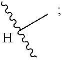
R² is 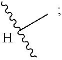
and
R³ is

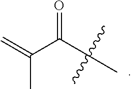 [SMILES: *C(=O)C(=C)C]

5. The compound of formula (I) of claim 1, wherein
R¹ is 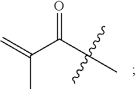 [SMILES: *C(=O)C(=C)C]
R² is 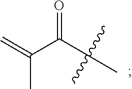 [SMILES: *C(=O)C(=C)C]
and
R³ is

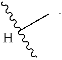

6. The compound of formula (I) of claim 1, wherein
R¹ is 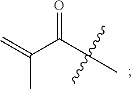 [SMILES: *C(=O)C(=C)C]
R² is 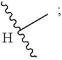
and
R³ is

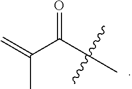 [SMILES: *C(=O)C(=C)C]

7. The compound of formula (I) of claim 1, wherein
R¹ is 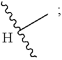
R² is 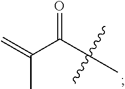 [SMILES: *C(=O)C(=C)C]
and
R³ is

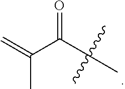 [SMILES: *C(=O)C(=C)C]

8. The compound of formula (I) of claim 1, wherein
R¹ is 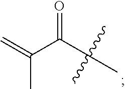 [SMILES: *C(=O)C(=C)C]
R² is 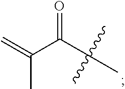 [SMILES: *C(=O)C(=C)C]
and
R³ is

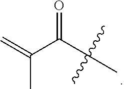 [SMILES: *C(=O)C(=C)C]

9. A method of synthesizing a pH-responsive polymer comprising:
maintaining a vessel at a reaction temperature;
mixing a collagen with a basic water in the vessel to form a first solution having a target pH;
stirring the first solution at the reaction temperature while:
beginning a reaction by adding methacrylic anhydride to the vessel to form a second solution;
conducting the reaction for a first time period in the absence of light while maintaining the second solution at the target pH by
obtaining a measured pH of the second solution at intervals during the first time period,
determining a difference between the measured pH and the target pH at each one of the intervals, and
adding a base to the vessel at each one of the intervals so that the measured pH equals the target pH; and
removing unreacted methacrylic anhydride from the second solution.

10. The method of claim 9, wherein maintaining the vessel at the reaction temperature comprises placing the vessel in a temperature bath.

11. The method of claim 9, wherein the reaction temperature is between approximately 18° C. and approximately 25° C.

12. The method of claim 9, wherein the target pH is 10.

13. The method of claim 9, wherein mixing the collagen with the basic water comprises:
dissolving the collagen in an acetic acid in the vessel;
adding water and sodium hydroxide to the vessel; and
stirring contents of the vessel to form the first solution.

14. The method of claim 13, wherein:
the collagen comprises a Type 1 rat tail collagen as a 5 mg/mL solution;
the acetic acid comprises a 20 mM aqueous acetic acid; and
the sodium hydroxide comprises a 2 M sodium hydroxide.

15. The method of claim 9, wherein mixing the collagen with the basic water comprises adding the basic water to the first solution dropwise at a flowrate of approximately 1 mL/min.

16. The method of claim 9, comprising stirring the first solution at approximately 600 rpm.

17. The method of claim 9, comprising adding the methacrylic anhydride dropwise at a flowrate of approximately 100 μL/min.

18. The method of claim 9, comprising stirring the second solution at approximately 600 rpm.

19. The method of claim 9, wherein the basic water has a pH of approximately 10.

20. The method of claim 19, wherein the base has a pH of approximately 14.

21. The method of claim 9, wherein the first time period is approximately 2 hours.

22. The method of claim 9, wherein each one of the intervals is approximately 10 minutes.

23. The method of claim 9, wherein the intervals occur multiple times during each hour of the first time period.

24. The method of claim 9, wherein removing the unreacted methacrylic anhydride comprises:
dialyzing the second solution;
freezing the second solution; and
lyophilizing the second solution.

25. The method of claim 24, wherein dialyzing the second solution comprises:
transferring the second solution to a dialysis membrane; and
removing the unreacted methacrylic anhydride by dialysis with the dialysis membrane.

26. The method of claim 25, wherein removing the unreacted methacrylic anhydride with the dialysis membrane comprises dialyzing the second solution against water, with the dialysis membrane, for a dialysis time period at a dialysis temperature.

27. The method of claim 26, wherein the dialysis temperature is 30° C.

28. The method of claim 26, wherein the dialysis temperature is approximately 30° C.

29. The method of any one of claims 26 to 28, wherein the dialysis time period is less than or equal to approximately 7 days.

30. The method of any one of claims 26 to 29, wherein the dialysis time period is at least approximately 5 days.

31. The method of any one of claims 26 to 30, comprising adjusting the dialysis time period relative to a volume of the second solution.

32. The method of any one of claims 24 to 31, wherein freezing the second solution comprises exposing the second solution to a freezing temperature equal to approximately −196° C. to create a frozen second solution.

33. The method of claim 32, wherein lyophilizing the second solution comprises placing the frozen second solution in a lyophilizer for a lyophilization time period.

34. The method of claim 33, comprising outputting a dry product of methacrylated collagen polymer from the lyophilizer.

35. The method of claim 34, wherein the lyophilization time period is less than or equal to approximately 7 days.

36. The method of claim 35, wherein the lyophilization time period is at least approximately 3 days.

37. The method of claim 36, comprising adjusting the lyophilization time period relative to a volume of the frozen second solution.

38. A method of synthesizing a pH responsive polymer comprising:
maintaining a vessel at a reaction temperature;
mixing a collagen with an acidic water in the vessel to form a first solution;
adding a neutral water to the vessel to form a second solution having a target pH;
stirring the second solution at the reaction temperature while
beginning a reaction by adding methacrylic anhydride to the vessel to form a third solution, and
conducting the reaction in the absence of light for a second time period; and
removing unreacted methacrylic anhydride from the third solution.

39. The method of claim 38, wherein maintaining the vessel at the reaction temperature comprises placing the vessel in a temperature bath.

40. The method of claim 38, wherein maintaining the vessel at the reaction temperature comprises placing the vessel in a temperature-controlled environment.

41. The method of claim 38, wherein the reaction temperature is between approximately 3° C. and approximately 6° C.

42. The method of claim 38, wherein the target pH is 7.5.

43. The method of claim 38, wherein the acidic water comprises a hydrochloric acid and mixing the mixing the collagen with the acidic water comprises:
dissolving the collagen in the hydrochloric acid; and
stirring the collagen and the acidic water in the vessel for a first time period to form the first solution.

44. The method of claim 43, wherein:
the collagen comprises a type 1 rat tail collagen;
the hydrochloric acid comprises a 10 mM aqueous hydrochloric acid; and
the first time period is approximately 24 hours.

45. The method of claim 38, wherein adding the neutral water comprises:
adding a sodium hydrogen phosphate to the first solution; and
stirring the sodium hydrogen phosphate and the first solution in the vessel to form the second solution.

46. The method of claim 45, wherein the sodium hydrogen phosphate comprises a 0.2 M aqueous sodium hydrogen phosphate dibasic.

47. The method of claim 45, comprising stirring the second solution in the vessel at a stir rate of approximately 600 rpm.

48. The method of claim 38, comprising adding the methacrylic anhydride at a rate of approximately 100 μL/min.

49. The method of claim 38, wherein the second time period is approximately 8 hours.

50. The method of claim 38, wherein removing the unreacted methacrylic anhydride comprises:
dialyzing the third solution;
freezing the third solution; and
lyophilizing the third solution.

51. The method of claim 50, wherein dialyzing the third solution comprises:
transferring the third solution to a dialysis membrane; and
removing the unreacted methacrylic anhydride by dialysis with the dialysis membrane.

52. The method of claim 51, wherein removing the unreacted methacrylic anhydride with the dialysis membrane comprises dialyzing the third solution against 10 mM HCl, with the dialysis membrane, for a dialysis time period at a dialysis temperature.

53. The method of claim 52, wherein the dialysis temperature is 4 C.

54. The method of claim 52, wherein the dialysis temperature is between 2° C. and 8° C.

55. The method of any one of claims 52 to 54, wherein the dialysis time period is equal to or less than approximately 3 days.

56. The method of any one of claims 52 to 55, wherein the dialysis time period is at least approximately 2 days.

57. The method of any one of claims 52 to 56, wherein freezing the third solution comprises exposing the third solution to a freezing temperature equal to approximately −196° C. to create a frozen third solution.

58. The method of claim 57, wherein lyophilizing the third solution comprises placing the frozen third solution in a lyophilizer for a lyophilization time period.

59. The method of claim 58, comprising outputting a dry product of methacrylated collagen polymer from the lyophilizer.

60. The method of claim 59, wherein the lyophilization time period is less than or equal to approximately 7 days.

61. The method of claim 60, wherein the lyophilization time period is at least approximately 3 days.

62. The method of claim 61, comprising adjusting the lyophilization time period relative to a volume of the frozen third solution.

63. A compound of formula (II) 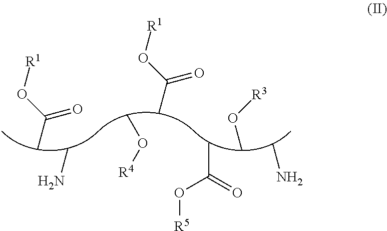
wherein:
the compound comprises an elastin backbone;
R¹, R², R³, R⁴, and R⁵ are selected from a group consisting of: 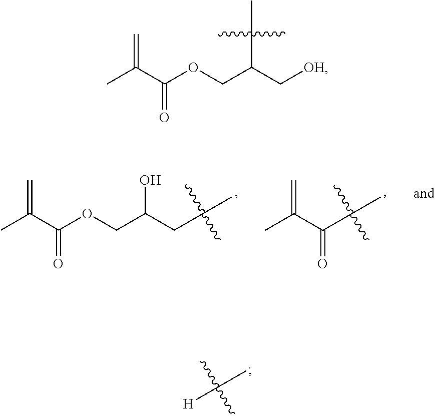
at least one of R¹, R², R³, R⁴, and R⁵ is not 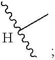
and
“ [symbol] ” indicates the point of attachment.

64. The compound of formula (II) of claim 63, wherein
R¹ is 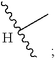
R² is 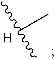
R³ is 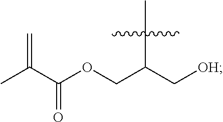 [SMILES: *C(CO)COC(=O)C(=C)C]
R⁴ is 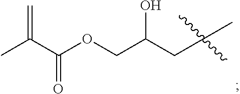 [SMILES: *CC(O)COC(=O)C(=C)C]
and
R⁵ is

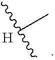

65. The compound of formula (II) of claim 63, wherein
R¹ is 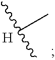
R² is 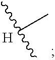
R³ is 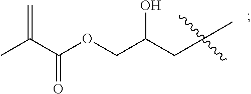 [SMILES: *CC(O)COC(=O)C(=C)C]
R⁴ is 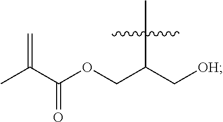 [SMILES: *C(CO)COC(=O)C(=C)C]
and
R⁵ is

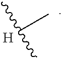

66. The compound of formula (II) of claim 63, wherein
R¹ is 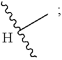
R² is 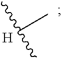
R³ is 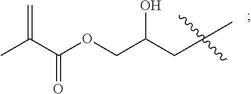 [SMILES: *CC(O)COC(=O)C(=C)C]
R⁴ is 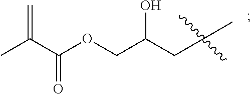 [SMILES: *CC(O)COC(=O)C(=C)C]
and
R⁵ is

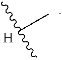

67. The compound of formula (II) of claim 63, wherein
R¹ is 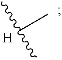
R² is 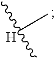
R³ is 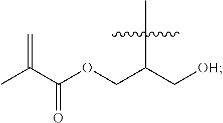 [SMILES: *C(CO)COC(=O)C(=C)C]
R⁴ is 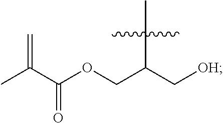 [SMILES: *C(CO)COC(=O)C(=C)C]
and
R⁵ is

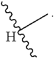

68. The compound of formula (II) of claim 63, wherein
R¹ is 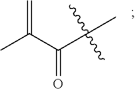 [SMILES: *C(=O)C(=C)C]
R² is 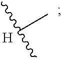
R³ is 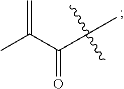 [SMILES: *C(=O)C(=C)C]
R⁴ is 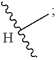
and
R⁵ is

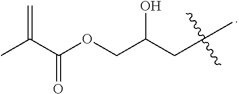 [SMILES: *CC(O)COC(=O)C(=C)C]

69. The compound of formula (II) of claim 63, wherein
R¹ is 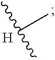
R² is 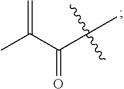 [SMILES: *C(=O)C(=C)C]
R³ is 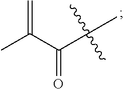 [SMILES: *C(=O)C(=C)C]
R⁴ is 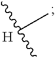
and
R⁵ is

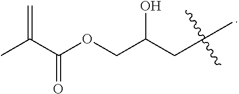 [SMILES: *CC(O)COC(=O)C(=C)C]

70. The compound of formula (II) of claim 63, wherein
R¹ is 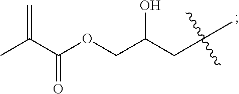 [SMILES: *CC(O)COC(=O)C(=C)C]
R² is 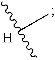
R³ is 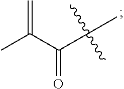 [SMILES: *C(=O)C(=C)C]
R⁴ is 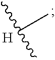
and
R⁵ is

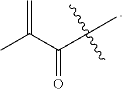 [SMILES: *C(=O)C(=C)C]

71. The compound of formula (II) of claim 63, wherein
R¹ is 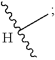
R² is 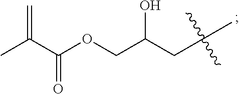 [SMILES: *CC(O)COC(=O)C(=C)C]
R³ is 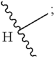
R⁴ is 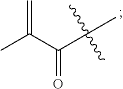 [SMILES: *C(=O)C(=C)C]
and
R⁵ is

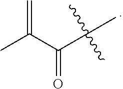 [SMILES: *C(=O)C(=C)C]

72. The compound of formula (II) of claim 63, wherein
R¹ is 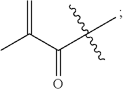 [SMILES: *C(=O)C(=C)C]
R² is 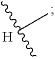
R³ is 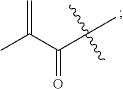 [SMILES: *C(=O)C(=C)C]
R⁴ is 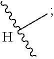
and
R⁵ is

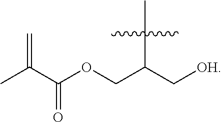 [SMILES: *C(CO)COC(=O)C(=C)C]

73. The compound of formula (II) of claim 63, wherein
R¹ is 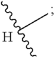
R² is 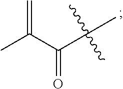 [SMILES: *C(=O)C(=C)C]
R³ is 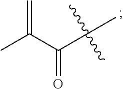 [SMILES: *C(=O)C(=C)C]
R⁴ is 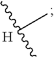
and
R⁵ is

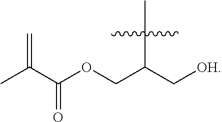 [SMILES: *C(CO)COC(=O)C(=C)C]

74. The compound of formula (II) of claim 63, wherein
R¹ is 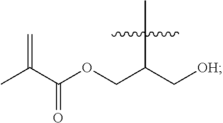 [SMILES: *C(CO)COC(=O)C(=C)C]
R² is 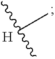
R³ is 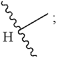
R⁴ is 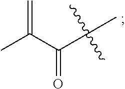 [SMILES: *C(=O)C(=C)C]
and
R⁵ is

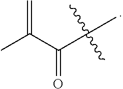 [SMILES: *C(=O)C(=C)C]

75. The compound of formula (II) of claim 63, wherein
R¹ is 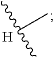
R² is 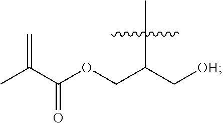 [SMILES: *C(CO)COC(=O)C(=C)C]
R³ is 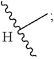
R⁴ is 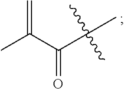 [SMILES: *C(=O)C(=C)C]
and
R⁵ is

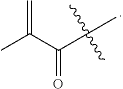 [SMILES: *C(=O)C(=C)C]

76. A method of synthesizing a pH-responsive polymer comprising:
maintaining a vessel at a reaction temperature;
mixing a hydrolyzed elastin with a basic water in the vessel to form a first solution having a first pH;
stirring the first solution at the reaction temperature while:
beginning a reaction by adding methacrylic anhydride to the vessel to form a second solution;
conducting the reaction for a first time period in the absence of light while maintaining the second solution at the target pH by
obtaining a measured pH of the second solution at intervals during the first time period,
determining a difference between the measured pH and the target pH at each one of the intervals, and
adding a base to the vessel at each one of the intervals so that the measured pH equals the target pH; and
removing unreacted methacrylic anhydride from the second solution.

77. The method of claim 76, wherein maintaining the vessel at the reaction temperature comprises placing the vessel in a temperature bath.

78. The method of claim 76, wherein the reaction temperature is between approximately 18° C. and approximately 25° C.

79. The method of claim 76, wherein the target pH is 10.

80. The method of claim 76, wherein mixing the hydrolyzed elastin with the basic water comprises:
dissolving the hydrolyzed elastin in the basic water in the vessel;
adding water and sodium hydroxide to the vessel; and
stirring the hydrolyzed elastin, the basic water, the water, and the sodium hydroxide in the vessel to form the first solution.

81. The method of claim 76, wherein mixing the hydrolyzed elastin with the basic water comprises adding the basic water to the first solution dropwise at a flowrate of approximately 1 mL/min.

82. The method of claim 76, comprising one or both of:
stirring the first solution at approximately 600 rpm; and
stirring the second solution at approximately 600 rpm.

83. The method of claim 76, comprising adding the methacrylic anhydride dropwise at a flowrate of approximately 100 μL/min.

84. The method of claim 76, wherein the basic water has a pH of approximately 10.

85. The method of claim 76, wherein the base has a pH of approximately 14.

86. The method of claim 76, wherein the base comprises 2 M sodium hydroxide.

87. The method of claim 76, wherein the first time period is approximately 2 hours.

88. The method of claim 76, wherein each one of the intervals is approximately 10 minutes.

89. The method of claim 76, wherein the intervals occur multiple times during each hour of the first time period.

90. The method of claim 76, wherein removing the unreacted methacrylic anhydride comprises:
dialyzing the second solution;
freezing the second solution; and
lyophilizing the second solution.

91. The method of claim 90, wherein dialyzing the second solution comprises:
transferring the second solution to a dialysis membrane; and
removing the unreacted methacrylic anhydride by dialysis with the dialysis membrane.

92. The method of claim 91, wherein removing the unreacted methacrylic anhydride with the dialysis membrane comprises dialyzing the second solution against water, with the dialysis membrane, for a dialysis time period at a dialysis temperature.

93. The method of claim 92, wherein the dialysis temperature is 30° C.

94. The method of claim 92, wherein the dialysis temperature is approximately 30° C.

95. The method of any one of claims 92 to 94, wherein the dialysis time period is less than or equal to approximately 7 days.

96. The method of any one of claims 92 to 95, wherein the dialysis time period is at least approximately 5 days.

97. The method of any one of claims 92 to 96, comprising adjusting the dialysis time period relative to a volume of the second solution.

98. The method of any one of claims 90 to 97, wherein freezing the second solution comprises exposing the second solution to a freezing temperature equal to approximately −196° C. to create a frozen second solution.

99. The method of claim 98, wherein lyophilizing the second solution comprises placing the frozen second solution in a lyophilizer for a lyophilization time period.

100. The method of claim 99, comprising outputting a dry product of methacrylated hydrolyzed elastin from the lyophilizer.

101. The method of claim 100, wherein the lyophilization time period is less than or equal to approximately 7 days.

102. The method of claim 101, wherein the lyophilization time period is at least approximately 3 days.

103. The method of claim 102, comprising adjusting the lyophilization time period relative to a volume of the frozen second solution.

104. The method of any one of claims 76 to 103, comprising synthesizing the hydrolyzed elastin.

105. The method of claim 104, wherein synthesizing the hydrolyzed elastin comprises:
suspending elastin in an aqueous oxalic acid solution to form a suspension;
refluxing the suspension for an initial time period at an initial temperature;
cooling the suspension to a cooled temperature after the initial time period;
centrifuging the suspension for a subsequent time period at the cooled temperature;
collecting a supernatant comprising the hydrolyzed elastin from the suspension;
collecting a precipitate comprising elastin from the suspension;
repeating the preceding steps with the precipitate to collect additional amounts of the hydrolyzed elastin; and
purifying the hydrolyzed elastin collected during the preceding steps.

106. The method of claim 105, wherein suspending the elastin comprises suspending 5 g of the elastin in 40 mL of a 0.25 M aqueous oxalic acid solution.

107. The method of claim 105, wherein:
the initial time period is approximately one hour;
the initial temperature is approximately 100° C.; and
the cooled temperature is approximately 4° C.

108. The method of claim 107, comprising centrifuging the suspension at 3750 rpm.

109. The method of claim 105, wherein purifying the hydrolyzed elastin comprises:
dialyzing the hydrolyzed elastin collected in the method of claim 105;
freezing the hydrolyzed elastin collected in the method of claim 105; and
lyophilizing the hydrolyzed elastin collected in the method of claim 105.

110. A compound of formula (III) 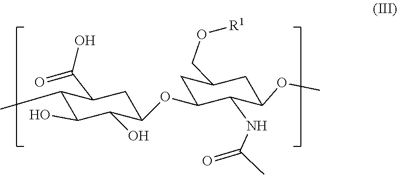
wherein:
the compound comprises a hyaluronic acid backbone;
R¹ is selected from a group consisting of: 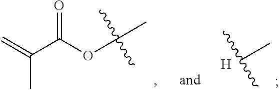
and
“ [symbol] ” indicates the point of attachment.

111. The compound of formula (III) of claim 110, wherein
R¹ is

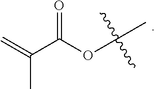 [SMILES: *OC(=O)C(=C)C]

112. The compound of formula (III) of claim 110, wherein
R¹ is

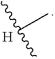

113. A method of synthesizing a pH-responsive polymer comprising:
maintaining a vessel at a reaction temperature;
mixing a hyaluronic acid with a basic water in the vessel for a first time period to form a first solution having a target pH;
stirring the first solution at the reaction temperature while
beginning a reaction by adding methacrylic anhydride to the vessel to form a second solution,
adding a base to the vessel to maintain the second solution at the target pH, and
conducting the reaction in the absence of light for a second time period while maintaining the second solution at the reaction temperature; and
removing unreacted methacrylic anhydride from the second solution.

114. The method of claim 113, wherein maintaining the vessel at the reaction temperature comprises placing the vessel in a temperature bath.

115. The method of claim 113, wherein the reaction temperature is approximately 4° C.

116. The method of claim 113, wherein the reaction temperature is between approximately 0° C. and approximately 10° C.

117. The method of claim 113, wherein the hyaluronic acid comprises a hyaluronic acid sodium salt.

118. The method of claim 113, wherein mixing the hyaluronic acid with the basic water comprises adding the basic water to the first solution dropwise at a flowrate of approximately 1 mL/min.

119. The method of claim 113, wherein the first time period is approximately 24 hours.

120. The method of claim 113, comprising stirring the first solution at approximately 600 rpm.

121. The method of claim 113, comprising adding the methacrylic anhydride dropwise at a flowrate of approximately 100 μL/min.

122. The method of claim 113, comprising stirring the second solution at approximately 600 rpm.

123. The method of claim 113, wherein the target pH is 10.

124. The method of claim 113, wherein the basic water has a pH of approximately 10.

125. The method of claim 113, wherein the base has a pH of approximately 14.

126. The method of claim 113, wherein the base comprises 5 M sodium hydroxide.

127. The method of claim 113, wherein the second time period is approximately 24 hours.

128. The method of claim 113, wherein removing the unreacted methacrylic anhydride comprises:
dialyzing the second solution;
freezing the second solution; and
lyophilizing the second solution.

129. The method of claim 128, wherein dialyzing the second solution comprises:
transferring the second solution to a dialysis membrane; and
removing the unreacted methacrylic anhydride by dialysis with the dialysis membrane.

130. The method of claim 129, wherein removing the unreacted methacrylic anhydride with the dialysis membrane comprises dialyzing the second solution against water, with the dialysis membrane, for a dialysis time period at a dialysis temperature.

131. The method of claim 130, wherein the dialysis temperature is 40° C.

132. The method of claim 130, wherein the dialysis temperature is approximately 40° C.

133. The method of any one of claims 130 to 132, wherein the dialysis time period is less than or equal to approximately 7 days.

134. The method of any one of claims 130 to 133, wherein the dialysis time period is at least approximately 5 days.

135. The method of any one of claims 130 to 134, comprising adjusting the dialysis time period relative to a volume of the second solution.

136. The method of any one of claims 128 to 135, wherein freezing the second solution comprises exposing the second solution to a freezing temperature equal to approximately −196° C. to create a frozen second solution.

137. The method of claim 136, wherein lyophilizing the second solution comprises placing the frozen second solution in a lyophilizer for a lyophilization time period.

138. The method of claim 128, comprising outputting a dry product of methacrylated hyaluronic acid polymer from the lyophilizer.

139. The method of claim 138, wherein the lyophilization time period is less than or equal to approximately 7 days.

140. The method of claim 139, wherein the lyophilization time period is at least approximately 3 days.

141. The method of claim 140, comprising adjusting the lyophilization time period relative to a volume of the frozen second solution.

142. A method of synthesizing a pH responsive polymer comprising:
maintaining a vessel at a reaction temperature;
mixing a hyaluronic acid with a water in the vessel for a first time period to form a first solution;
adding a basic water to the vessel to form a second solution having a target pH;
stirring the second solution in the vessel at the reaction temperature while
beginning a reaction by adding methacrylic anhydride to the vessel to form a third solution,
adding a base to the vessel to maintain the third solution at a target pH, and
conducting the reaction in the absence of light for a second time period; and
removing unreacted methacrylic anhydride from the third solution.

143. The method of claim 142, wherein maintaining the vessel at the reaction temperature comprises placing the vessel in a temperature bath.

144. The method of claim 142, wherein maintaining the vessel at the reaction temperature comprises placing the vessel in a temperature-controlled environment.

145. The method of claim 142, wherein the reaction temperature is approximately 4° C.

146. The method of claim 142, wherein the reaction temperature is between approximately 2° C. and approximately 10° C.

147. The method of claim 142, wherein mixing the hyaluronic acid with the water comprises:
dissolving the hyaluronic acid in the water in the vessel; and
stirring the hyaluronic acid and the water in the vessel for a first time period to form the first solution.

148. The method of claim 147, wherein:
the hyaluronic acid comprises a hyaluronic acid sodium salt; and
the first time period is approximately 24 hours.

149. The method of claim 142, wherein adding the basic water comprises:
adding sodium hydroxide to the first solution; and
stirring the sodium hydroxide and the first solution in the vessel to form the second solution.

150. The method of claim 149, wherein the sodium hydroxide comprises 5M aqueous sodium hydroxide.

151. The method of claim 142, wherein the target pH is 8.5.

152. The method of claim 149, comprising stirring the second solution at approximately 600 rpm.

153. The method of claim 142, comprising adding the methacrylic anhydride at a rate of approximately 100 μL/min.

154. The method of claim 142, wherein the base has a pH of approximately 14.

155. The method of claim 142, wherein the base comprises 5M sodium hydroxide.

156. The method of claim 142, comprising conducting the reaction for a time period of approximately 24 hours.

157. The method of claim 142, wherein removing the unreacted methacrylic anhydride comprises:
dialyzing the third solution;
freezing the third solution; and
lyophilizing the third solution.

158. The method of claim 157, wherein dialyzing the third solution comprises:
transferring the third solution to a dialysis membrane; and
removing the unreacted methacrylic anhydride by dialysis with the dialysis membrane.

159. The method of claim 158, wherein removing the unreacted methacrylic anhydride with the dialysis membrane comprises dialyzing the third solution against water, with the dialysis membrane, for a dialysis time period at a dialysis temperature.

160. The method of claim 159, wherein the dialysis temperature is 40° C.

161. The method of claim 159, wherein the dialysis temperature is approximately 40° C.

162. The method of any one of claims 159 to 161, wherein the dialysis time period is equal to or less than approximately 3 days.

163. The method of any one of claims 159 to 162, wherein the dialysis time period is at least approximately 2 days.

164. The method of any one of claims 159 to 163, wherein freezing the third solution comprises exposing the third solution to a freezing temperature equal to approximately −196° C. to create a frozen third solution.

165. The method of claim 164, wherein lyophilizing the third solution comprises placing the frozen third solution in a lyophilizer for a lyophilization time period.

166. The method of claim 165, comprising outputting a dry product of methacrylated hyaluronic acid polymer from the lyophilizer.

167. The method of claim 166, wherein the lyophilization time period is less than or equal to approximately 7 days.

168. The method of claim 167, wherein the lyophilization time period is at least approximately 3 days.

169. The method of claim 168, comprising adjusting the lyophilization time period relative to a volume of the frozen third solution.

170. A method of synthesizing a pH responsive polymer comprising:
maintaining a vessel at a reaction temperature;
mixing a hyaluronic acid with a buffer in the vessel to form a first solution having a target pH;
stirring the first solution at the reaction temperature while
beginning a reaction by adding methacrylic anhydride to the vessel to form a second solution, and
conducting the reaction in the absence of light; and
removing unreacted methacrylic anhydride from the second solution.

171. The method of claim 170, wherein maintaining the vessel at the reaction temperature comprises placing the vessel in a temperature bath.

172. The method of claim 170, wherein maintaining the vessel at the reaction temperature comprises placing the vessel in a temperature-controlled environment.

173. The method of claim 170, wherein the reaction temperature is approximately 4° C.

174. The method of claim 170, wherein the reaction temperature is between approximately 2° C. and approximately 10° C.

175. The method of claim 170, wherein the target pH is 8.5.

176. The method of claim 170, wherein the buffer comprises a sodium carbonate buffer and mixing the hyaluronic acid with the buffer comprises:
dissolving the hyaluronic acid in the sodium carbonate buffer in the vessel; and
stirring the hyaluronic acid and the sodium carbonate buffer in the vessel for a first time period to form the first solution.

177. The method of claim 176, wherein:
the hyaluronic acid comprises a hyaluronic acid sodium salt;
the sodium carbonate buffer comprises a bicarbonate aqueous solution; and
the first time period is approximately 24 hours.

178. The method of claim 176, comprising stirring the first solution at approximately 600 rpm.

179. The method of claim 170, comprising adding the methacrylic anhydride at a rate of approximately 100 μL/min.

180. The method of claim 170, comprising conducting the reaction for a time period of approximately 24 hours.

181. The method of claim 170, wherein removing the unreacted methacrylic anhydride comprises:
dialyzing the second solution;
freezing the second solution; and
lyophilizing the second solution.

182. The method of claim 181, wherein dialyzing the second solution comprises:
transferring the second solution to a dialysis membrane; and
removing the unreacted methacrylic anhydride by dialysis with the dialysis membrane

183. The method of claim 182, wherein removing the unreacted methacrylic anhydride with the dialysis membrane comprises dialyzing the second solution against water, with the dialysis membrane, for a dialysis time period at a dialysis temperature.

184. The method of claim 183, wherein the dialysis temperature is 40° C.

185. The method of claim 183, wherein the dialysis temperature is approximately 40° C.

186. The method of any one of claims 183 to 185, wherein the dialysis time period is equal to or less than approximately three days.

187. The method of any one of claims 183 to 186, wherein the dialysis time period is at least approximately 2 days.

188. The method of any one of claims 183 to 187, wherein freezing the second solution comprises exposing the second solution to a freezing temperature equal to approximately −196° C. to create a frozen second solution.

189. The method of claim 188, wherein lyophilizing the second solution comprises placing the frozen second solution in a lyophilizer for a lyophilization time period.

190. The method of claim 189, comprising outputting a dry product of methacrylated hyaluronic acid polymer from the lyophilizer.

191. The method of claim 190, wherein the lyophilization time period is less than or equal to approximately 7 days.

192. The method of claim 191, wherein the lyophilization time period is at least approximately 3 days.

193. The method of claim 192, comprising adjusting the lyophilization time period relative to a volume of the frozen second solution.

194. A compound of formula (IV) 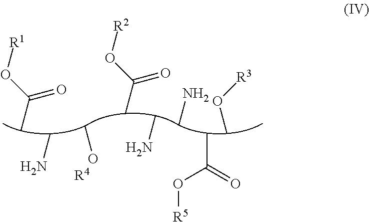
wherein:
the compound comprises a gelatin backbone;
R¹, R², R³, R⁴, and R⁵ are selected from a group consisting of: 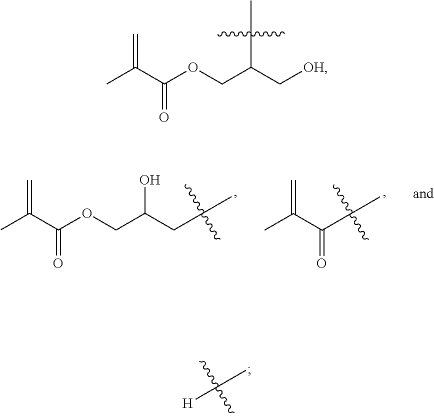
at least one of R¹, R², R³, R⁴, and R⁵ is not 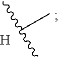
and
“ [symbol] ” indicates the point of attachment.

195. The compound of formula (IV) of claim 194, wherein
R¹ is 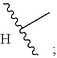
R² is 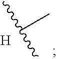
R³ is 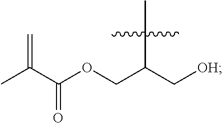 [SMILES: *C(CO)COC(=O)C(=C)C]
R⁴ is 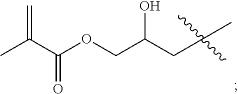 [SMILES: *CC(O)COC(=O)C(=C)C]
and
R⁵ is

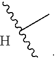

196. The compound of formula (IV) of claim 194, wherein
R¹ is 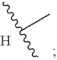
R² is 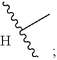
R³ is 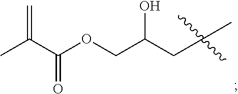 [SMILES: *CC(O)COC(=O)C(=C)C]
R⁴ is 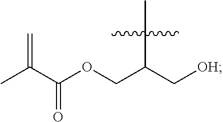 [SMILES: *C(CO)COC(=O)C(=C)C]
and
R⁵ is

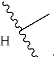

197. The compound of formula (IV) of claim 194, wherein
R¹ is 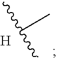
R² is 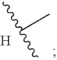
R³ is 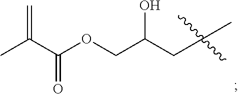 [SMILES: *CC(O)COC(=O)C(=C)C]
R⁴ is 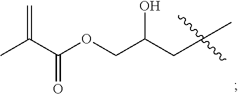 [SMILES: *CC(O)COC(=O)C(=C)C]
and
R⁵ is

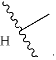

198. The compound of formula (IV) of claim 194, wherein
R¹ is 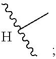
R² is 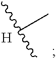
R³ is 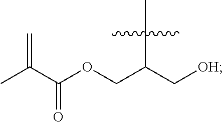 [SMILES: *C(CO)COC(=O)C(=C)C]
R⁴ is 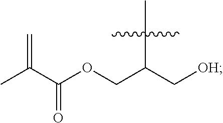 [SMILES: *C(CO)COC(=O)C(=C)C]
and
R⁵ is

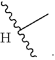

199. The compound of formula (IV) of claim 194, wherein
R¹ is 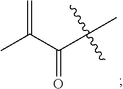 [SMILES: *C(=O)C(=C)C]
R² is 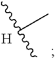
R³ is 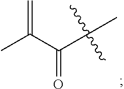 [SMILES: *C(=O)C(=C)C]
R⁴ is 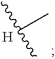
and
R⁵ is

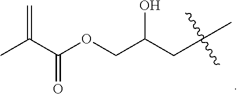 [SMILES: *CC(O)COC(=O)C(=C)C]

200. The compound of formula (IV) of claim 194, wherein
R¹ is 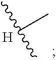
R² is 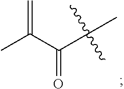 [SMILES: *C(=O)C(=C)C]
R³ is 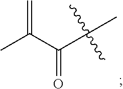 [SMILES: *C(=O)C(=C)C]
R⁴ is 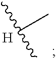
and
R⁵ is

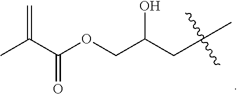 [SMILES: *CC(O)COC(=O)C(=C)C]

201. The compound of formula (IV) of claim 194, wherein
R¹ is 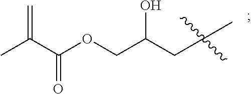 [SMILES: *CC(O)COC(=O)C(=C)C]
R² is 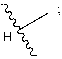
R³ is 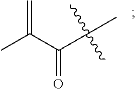 [SMILES: *C(=O)C(=C)C]
R⁴ is 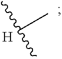
and
R⁵ is

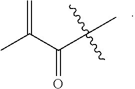 [SMILES: *C(=O)C(=C)C]

202. The compound of formula (IV) of claim 194, wherein
R¹ is 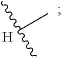
R² is 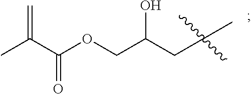 [SMILES: *CC(O)COC(=O)C(=C)C]
R³ is 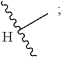
R⁴ is 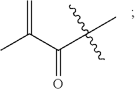 [SMILES: *C(=O)C(=C)C]
and
R⁵ is

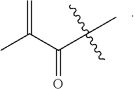 [SMILES: *C(=O)C(=C)C]

203. The compound of formula (IV) of claim 194, wherein
R¹ is 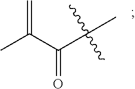 [SMILES: *C(=O)C(=C)C]
R² is 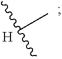
R³ is 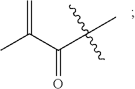 [SMILES: *C(=O)C(=C)C]
R⁴ is 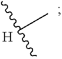
and
R⁵ is

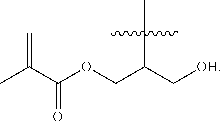 [SMILES: *C(CO)COC(=O)C(=C)C]

204. The compound of formula (IV) of claim 194, wherein
R¹ is 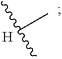
R² is 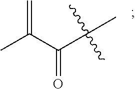 [SMILES: *C(=O)C(=C)C]
R³ is 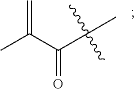 [SMILES: *C(=O)C(=C)C]
R⁴ is 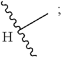
and
R⁵ is

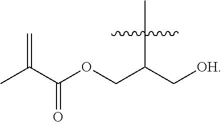 [SMILES: *C(CO)COC(=O)C(=C)C]

205. The compound of formula (IV) of claim 194, wherein
R¹ is 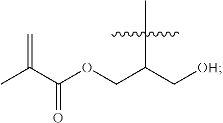 [SMILES: *C(CO)COC(=O)C(=C)C]
R² is 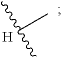
R³ is 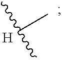
R⁴ is 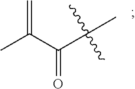 [SMILES: *C(=O)C(=C)C]
and
R⁵ is

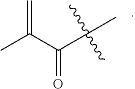 [SMILES: *C(=O)C(=C)C]

206. The compound of formula (IV) of claim 194, wherein
R¹ is 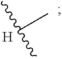
R² is 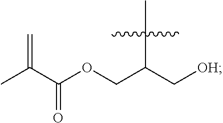 [SMILES: *C(CO)COC(=O)C(=C)C]
R³ is 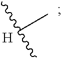
R⁴ is 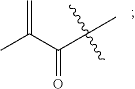 [SMILES: *C(=O)C(=C)C]
and
R⁵ is

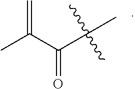 [SMILES: *C(=O)C(=C)C]

207. A method of synthesizing a pH-responsive polymer comprising:
maintaining a vessel at a reaction temperature;
mixing a gelatin with an acidic water in the vessel to form a first solution having a target pH;
stirring the first solution at the reaction temperature while:
adding an acid to the vessel to maintain the first solution at the target pH;
beginning a reaction by adding glycidyl methacrylate to the first solution in the vessel in aliquots for a first time period to form a second solution;
conducting the reaction in the absence of light for a second time period while maintaining the second solution at the target pH by
obtaining a measured pH of the second solution at intervals during the second time period,
determining a difference between the measured pH and the target pH at each one of the intervals, and
adding additional acid to the second solution at each one of the intervals so that the measured pH equals the target pH;
conducting the reaction in the absence of light for a third time period without maintaining the second solution at the target pH;
adding additional acidic water to the second solution after the third time period; and
removing unreacted glycidyl methacrylate from the second solution.

208. The method of claim 207, wherein maintaining the vessel at the reaction temperature comprises placing the vessel in a temperature bath.

209. The method of claim 207, wherein the reaction temperature is approximately 40° C.

210. The method of claim 207, wherein the reaction temperature is between approximately 40° C. and approximately 50° C.

211. The method of claim 207, wherein the target pH is 3.5.

212. The method of claim 207, wherein the gelatin comprises Gelatin Type A

213. The method of claim 207, comprising stirring the first solution at a rate of 800 rpm.

214. The method of claim 207, wherein the acidic water and the additional acidic water have a pH of approximately 3.5.

215. The method of claim 207, wherein the acid has a pH of approximately zero (0).

216. The method of claim 207, wherein the first time period is approximately 2.5 hours.

217. The method of claim 207, wherein beginning the reaction comprises adding the glycidyl methacrylate to the first solution dropwise at a flowrate of approximately 700 μL/min in equal-volume aliquots at first intervals during the first time period.

218. The method of claim 217, wherein each one of the first intervals is approximately 30 minutes.

219. The method of claim 207, wherein the second time period is approximately 12 hours.

220. The method of claim 207, wherein the target pH is 3.0 or 3.5.

221. The method of claim 207, wherein each one of the intervals is approximately 30 minutes.

222. The method of claim 207, wherein the third time period is approximately 12 hours.

223. The method of claim 207, wherein removing the unreacted glycidyl methacrylate comprises:
dialyzing the second solution;
freezing the second solution; and
lyophilizing the second solution.

224. The method of claim 223, wherein dialyzing the second solution comprises:
transferring the second solution to a dialysis membrane; and
removing the unreacted methacrylic anhydride by dialysis with the dialysis membrane.

225. The method of claim 224, wherein removing the unreacted methacrylic anhydride with the dialysis membrane comprises dialyzing the second solution against water, with the dialysis membrane, for a dialysis time period at a dialysis temperature.

226. The method of claim 225, wherein the dialysis temperature is 40° C.

227. The method of claim 225, wherein the dialysis temperature is approximately 40° C.

228. The method of any one of claims 225 to 227, wherein the dialysis time period is less than or equal to approximately 7 days.

229. The method of any one of claims 225 to 228, wherein the dialysis time period is at least approximately 5 days.

230. The method of any one of claims 225 to 229, comprising adjusting the dialysis time period relative to a volume of the second solution.

231. The method of any one of claims 223 to 230, wherein freezing the second solution comprises exposing the second solution to a freezing temperature equal to approximately −196° C. to create a frozen second solution.

232. The method of claim 231, wherein lyophilizing the second solution comprises placing the frozen second solution in a lyophilizer for a lyophilization time period.

233. The method of claim 232, comprising outputting a dry product of Polymer A from the lyophilizer.

234. The method of claim 233, wherein the lyophilization time period is less than or equal to approximately 5 days.

235. The method of claim 234, wherein the lyophilization time period is at least approximately 3 days.

236. The method of claim 235, comprising adjusting the lyophilization time period relative to a volume of the frozen second solution.

237. A bioink comprising:
one or more of pH-responsive polymers;
a photoinitiator; and
a solvent.

238. The bioink of claim 237, wherein the one or more pH-responsive polymers are selected from a group comprising:
the compound of any one of claims 1 to 8;
the compound of any one of claims 63 to 75;
the compound of any one of claims 110 to 112; and
the compound of any one of claims 194 to 206.

239. The bioink of claim 238, comprising:
10% w/v of GelMA; and
0.1% w/v of the compound of any one of claims 1 to 8.

240. The bioink of claim 237, wherein the one or more pH-responsive polymers comprise at least two of:
the compound of any one of claims 1 to 8;
the compound of any one of claims 63 to 75;
the compound of any one of claims 110 to 112; and
the compound of any one of claims 194 to 206.

241. The bioink of claim 240, comprising:
7% w/v of GelMA;
0.1% w/v of the compound of any one of claims 1 to 8; and
0.05% w/v of the compound of any one of claims 110 to 112.

242. The bioink of claim 240, comprising:
9% w/v of GelMA;
0.1% w/v of the compound of any one of claims 1 to 8; and
0.05% w/v of the compound of any one of claims 110 to 112.

243. The bioink of claim 240, comprising:
9% w/v of Polymer B;
0.2% w/v of the compound of any one of claims 1 to 8; and
0.1% w/v of the compound of any one of claims 110 to 112.

244. The bioink of claim 237, wherein the one or more pH-responsive polymers comprise at least three of:
the compound of any one of claims 1 to 8;
the compound of any one of claims 63 to 75;
the compound of any one of claims 110 to 112; and
the compound of any one of claims 194 to 206.

245. The bioink of claim 237, wherein the one or more pH-responsive polymers comprise:
the compound of any one of claims 1 to 8;
the compound of any one of claims 63 to 75;
the compound of any one of claims 110 to 112; and
the compound of any one of claims 194 to 206.

246. The bioink of claim 245, comprising:
0.1% w/v of the compound of any one of claims 1 to 8;
0.1% w/v of the compound of any one of claims 63 to 75;
0.05% w/v of the compound of any one of claims 110 to 112; and
12.0% w/v of the compound of any one of claims 194 to 206.

247. The bioink of claim 245, comprising:
0.1% w/v of the compound of any one of claims 1 to 8;
0.1% w/v of the compound of any one of claims 63 to 75;
0.1% w/v of the compound of any one of claims 110 to 112; and
11.0% w/v of the compound of any one of claims 194 to 206.

248. The bioink of claim 245, comprising:
0.1% w/v of the compound of any one of claims 1 to 8;
0.5% w/v of the compound of any one of claims 63 to 75;
0.1% w/v of the compound of any one of claims 110 to 112; and
15.0% w/v of the compound of any one of claims 194 to 206.

249. The bioink of claim 245, comprising:
0.1% w/v of the compound of any one of claims 1 to 8;
0.5% w/v of the compound of any one of claims 63 to 75;
0.5% w/v of the compound of any one of claims 110 to 112; and
17.5% w/v of the compound of any one of claims 194 to 206.

250. The bioink of claim 245, comprising:
0.1% w/v of the compound of any one of claims 1 to 8;
0.1% w/v of the compound of any one of claims 63 to 75;
0.05% w/v of the compound of any one of claims 110 to 112; and
7.0% w/v of the compound of any one of claims 194 to 206.

251. The bioink of claim 245, comprising:
0.1% w/v of the compound of any one of claims 1 to 8;
0.5% w/v of the compound of any one of claims 63 to 75;
0.5% w/v of the compound of any one of claims 110 to 112; and
13.0% w/v of the compound of any one of claims 194 to 206.

252. The bioink of any one of claims 237 to 251, wherein the photoinitiator is selected from a group comprising:
lithium phenyl-2,4,6-trimethylbenzoylphosphinate (LAP);
2-hydroxy-4′-(2-hydroxyethoxy)-2-methylpropiophenone (Irgacure I2959);
tetrapotassium-4,4′-(1,2-ethenediyl)bis[2-(3-sulfophenyl)diazenesulfonate](AS7);
E2CK; and
P2CK.

253. The bioink of any one of claims 237 to 252, wherein solvent comprises one of:
phosphate buffered saline;
a cell culture media; or
water.

254. The bioink of any one of claims 237, 238, 240, and 244 to 253, comprising one of:
a type A GelMA; and
a type B GelMA.

255. A method of preparing bioinks comprising:
mixing one or more pH-responsive polymers into a mixture;
forming a suspension by adding a photoinitiator solution to the mixture, the photoinitiator solution comprising a photoinitiator and a solvent; and
mixing the suspension at a mixing rate and heating the suspension a temperature to fully dissolve components of the suspension.

256. The method of claim 255, wherein the mixture comprises a mixture of solids.

257. The method of claim 255, wherein the mixture comprises a partially dissolved suspension of polymers in a phosphate buffered saline or a cell culture media.

258. The method of claim 255, wherein:
the photoinitiator solution comprises a photoinitiator dissolved in the solvent; and
the solvent comprises one of a phosphate buffered saline, a cell culture media, or water.

259. The method of claim 255, wherein the photoinitiator is selected from a group comprising:
lithium phenyl-2,4,6-trimethylbenzoylphosphinate (LAP);
2-hydroxy-4′-(2-hydroxyethoxy)-2-methylpropiophenone (Irgacure I2959);
tetrapotassium-4,4′-(1,2-ethenediyl)bis[2-(3-sulfophenyl)diazenesulfonate](AS7);
E2CK; and
P2CK.

260. The method of claim 255, wherein mixing the suspension comprises stirring or vortex mixing.

261. The method of claim 255, comprising:
mixing the suspension at a first mixing rate of approximately 200 rpm to a form the mixture; and
mixing the mixture a second mixing rate of between approximately 800 rpm and approximately 1500 rpm.

262. The method of claim 255, wherein the temperature is at least 20° C.

263. The method of claim 255, wherein the temperature is 50° C.

264. The method of claim 255, wherein the temperature is between approximately 20° C. and approximately 50° C.

265. A method of determining characteristics of a bioink, the method comprising:
preparing a hydrogel pellet of photo-crosslinked bioink;
soaking the hydrogel pellet in a fluorescein sodium salt solution;
drop-casting fluorescent polystyrene particles onto the surface of the hydrogel pellet;
measuring elastic parameters of the hydrogel pellet; and
calculating an elastic modulus of the hydrogel pellet.

266. The method of claim 265, wherein the hydrogel pellet comprises a diameter of 1 cm.

267. The method of claim 265, wherein the hydrogel pellet comprises a thickness of approximately 300 to 700 μm.

268. The method of claim 265, wherein preparing the hydrogel pellet comprises:
drop-casting a droplet of the bioink onto a PTFE surface;
covering the droplet with a glass coverslip and spacer; and
exposing the glass coverslip to a UV light for a first time period.

269. The method of claim 268, wherein UV light comprises 365 nm.

270. The method of claim 268, wherein the first time period is approximately 3 minutes.

271. The method of claim 265, wherein soaking the hydrogel pellet in the fluorescein sodium salt solution comprises:
transferring the hydrogel pellet and the cover slip to a well-plate;
adding an amount of fluorescein sodium salt to the well-plate;
adding an amount of phosphate buffered saline to the well-plate; and
maintaining the well-plate at a temperature for a second time period.

272. The method of claim 271, wherein the fluorescein sodium salt comprises 35 μM fluorescein sodium salt in a phosphate buffered saline.

273. The method of claim 271, wherein the temperature is approximately 25° C.

274. The method of claim 271, wherein the temperature is between 20° C. and 30° C.

275. The method of claim 271, wherein the second time period is approximately 16 hours.

276. The method of claim 268, wherein drop-casting the fluorescent polystyrene particles onto the hydrogel pellet comprises:
transferring the hydrogel pellet and coverslip to a microscope sample holder;
drop-casting the fluorescent polystyrene particles onto the hydrogel pellet; and
allowing the fluorescent polystyrene particles to settle for a third time period.

277. The method of claim 276, wherein the fluorescent polystyrene particles are suspended in water.

278. The method of claim 276, wherein the third time period is at least 5 minutes.

279. The method of claim 276, wherein measuring the elastic parameters of the hydrogel pellet comprises:
placing an indenter onto the hydrogel pellet;
measuring a depth of an indention made on the hydrogel pellet; and
measuring a thickness of the hydrogel pellet.

280. The method of claim 279, wherein:
a first ratio of a radius of the indenter to the thickness of the hydrogel pellet is between 0.3 and 12; and
a second ratio of the depth of indentation to the thickness of the hydrogel pellet is less than 0.6.

281. The method of claim 279, comprising using laser scanning confocal microscopy to measure one or both of:
the depth of the indention; and
the thickness of the hydrogel pellet.

282. The method of claim 265, wherein calculating the elastic modulus comprises using a modified Hertz model based on variables comprising:
the elastic parameters of the hydrogel pellet;
a force applied by the indenter;
acceleration due to gravity;
a density of the indenter; and
a density of a medium.

283. The method of claim 282, wherein the medium is a phosphate buffered saline.
